(19) United States
(12) Patent Application Publication
(10) Pub. No.: US 2025/0049815 A1
(43) Pub. Date: Feb. 13, 2025

(54) 25-HYDROXYCHOLESTEROL (25HC), CRYAB AGGREGATION INHIBITOR TO AMELIORIATE VASCULAR STIFFNESS

(71) Applicant: Buck Institute for Research on Aging, Novato, CA (US)

(72) Inventor: Simon Melov, Petaluma, CA (US)

(21) Appl. No.: 18/294,121

(22) PCT Filed: Aug. 2, 2022
(86) PCT No.: PCT/US22/39181

§ 371 Date: Jan. 31, 2024

Related U.S. Application Data
(60) Provisional application No. 63/228,575, filed on Aug. 2, 2021.

(51) Int. Cl. A61K 31/575; A61K 31/496; A61K 31/713; A61P 9/08

(52) U.S. Cl. CPC A61K 31/575; A61K 31/496; A61K 31/713; A61P 9/08

(57) ABSTRACT

Methods and compositions are provided herein that pertain to the discovery that the crystallin alpha B (CRYAB) gene and gene product provides an effective target for senolytic agents. In certain embodiments, methods of selectively killing one or more senescent cells in a subject in need thereof are provided where the method involves administering to the subject an effective amount of an agent that inhibits expression and/or aggregation of a CRYAB protein. In certain embodiments administration of the senolytic agent(s) describes herein decreases vascular stiffness, e.g., as measured by pulse wave velocity.

### CROSS-REFERENCE TO RELATED APPLICATIONS

This application claims priority to and benefit of U.S. Ser. No. 63/228,575, filed on Aug. 2, 2021, which is incorporated herein by reference in its entirety for all purposes.

### STATEMENT OF GOVERNMENTAL SUPPORT

[Not Applicable]

### INCORPORATION BY REFERENCE OF SEQUENCE LISTING PROVIDED AS A TEXT FILE

[Not Applicable]

### BACKGROUND

Senescent cells accumulate in tissues and organs of individuals as they age and are found at sites of age-related pathologies. Senescent cells are believed important for inhibiting the proliferation of dysfunctional or damaged cells and particularly for constraining the development of malignancy (see, e.g., Campisi et al. (2011) Curr. Opin. Genet. Dev., 21: 107-12; Campisi et al. (2001) Trends Cell Biol., 11: S27-31; Prieur et al. (2008) Curr. Opin. Cell Biol., 20: 150-55; and the like). The presence of senescent cells in an individual may contribute to aging and aging-related dysfunction (see, e.g., Campisi (2005) Cell, 120: 513-522; Gorgoulis et al. (2019) Cell, 179: 813-827; and the like).

Cellular senescence is a multi-faceted response to damage, stress and certain physiological signals that arrests cell proliferation, essentially irreversibly. It also activates the transcription and secretion of numerous pro-inflammatory cytokines, chemokines, growth factors and proteases, termed the Senescence Associated Secretory Phenotype (SASP). Senescent cells increase as a consequence of genotoxic and radiotherapy and/or cytotoxic anti-cancer therapies, and we have previously shown in mice that genetically ablating senescent cells ameliorates many deleterious outcomes of these therapies. The chemotherapeutic agent doxorubicin (DOXO) is widely used in the clinic, and DOXO induces senescence and a SASP in many tissues, including the vasculature and heart, and also impairs cardiac and vascular function similar to that seen with increased age (see, e.g., Demaria et al. (2017) Cancer Discov., 2: 165-176; Gorgoulis et al. (2019) Cell, 179: 813-827; and the like).

However, the number of senescent cells arising as a consequence of anti-cancer therapy is low, and characterizing such cells has proved to be problematic. In addition, senescent cells are heterogeneous, with SASPs that vary with cell type, senescence inducer and timing. Single cell profiling is a comparatively new technique that can identify the gene expression pattern of individual cells in a population. Therefore, applying the technique of single cell profiling to cells exposed to a chemotherapeutic such as DOXO facilitates the identification of novel genes which may be induced as a consequence of such exposure, and be masked using a bulk approach which averages all cells in the population together. Such genes expressed in a small population of senescent cells in a tissue may drive senescence mediated outcomes, and therefore eliminating cells that express these genes may be therapeutically beneficial to the patient undergoing chemotherapy, and more generally therapeutically or prophylactically beneficial to subjects suffering effects of aging due to the accumulation of senescent cells. However, identifying and developing treatments for such diseases and conditions by selective elimination of senescent cells has been an arduous undertaking.

### SUMMARY

In various embodiments, methods and compositions are provided for selectively killing one or more senescent cells in a subject in need thereof. The methods exploit the identification of the crystallin alpha B (CRYAB) gene as a target for senolytic agents. Additionally, in certain embodiments senolytic agents including, but not limited to agents that inhibit the expression and/or activity of CRYAB are used to decrease vascular stiffness and/or to slow or prevent increases in vascular stiffness (e.g., vascular stiffness associated with age and/or disease).

Accordingly, various embodiments provided herein may include, but need not be limited to, one or more of the following:

Various embodiments provided herein may include, but need not be limited to, one or more of the following:

Embodiment 1: A method of preventing or slowing an increase in vascular stiffness or of decreasing vascular stiffness in a subject, said method comprising: administering to said subject an effective amount of a senolytic agent.

Embodiment 2: The method of embodiment 1, wherein said method slows or prevents an increase in vascular stiffness in a subject.

Embodiment 3: The method of embodiment 2, wherein said method slows or prevents an increase in arterial stiffness in a subject.

Embodiment 4: The method of embodiment 3, wherein said method slows or prevents an increase in aortic stiffness in a subject.

Embodiment 5: The method according to any one of embodiments 1-4, wherein said method slows or prevents an increase in pulse wave velocity in said subject.

Embodiment 6: The method of embodiment 1, wherein said method decreases vascular stiffness in a subject.

Embodiment 7: The method of embodiment 6, wherein said method decreases arterial stiffness in a subject.

Embodiment 8: The method of embodiment 7, wherein said method decreases aortic stiffness in a subject.

Embodiment 9: The method according to any one of embodiments 1, and 6-8, wherein said method decreases pulse wave velocity in said subject.

Embodiment 10: The method according to any one of embodiments 1-5, wherein said method slows or stops a decrease in Windkessel effect, and/or slows or stops increased load on heart, and/or slows or stops increasing resting heart rate, and/or slows or stops decreases in the duration of systole with an accompanying increase in duration of diastole.

Embodiment 11: The method of embodiment 10, wherein said method slows or stops a decrease in Windkessel effect.

Embodiment 12: The method according to any one of embodiments 1, and 6-9, wherein said method improves Windkessel effect, and/or reduces load on heart, and/or lowers resting heart rate, and/or lowers duration of systole with an accompanying increase in duration of diastole in said subject.

Embodiment 13: The method of embodiment 12, wherein said method improves Windkessel effect in said subject.

Embodiment 14: The method according to any one of embodiments 1-12, wherein said senolytic agent is a senolytic agent that inhibits expression and/or aggregation of a CRYAB protein.

Embodiment 15: The method of embodiments 14, wherein said senolytic agent is selected from the group consisting of a small organic molecule, an inhibitory nucleic acid, an antibody, a CRISPR/Cas system, a zinc finger nuclease (ZFN), and a transcription activator-like effector nuclease (TALEN).

Embodiment 16: The method according to any one of embodiments 1-15, wherein said senolytic agent comprises a small organic molecule.

Embodiment 17: The method according to any one of embodiments 1-16, wherein said agent is selected from the group consisting of 25-hyddroxycholesterol (25HC), 24(S)-Hydroxycholesterol (24(S)HC), 27-Hydroxycholesterol (27HC), 22(R)-Hydroxycholesterol (22(R)HC), 7α-Hydroxycholesterol (7αHC), 7β-Hydroxycholesterol (7βHC), Calcifediol 25-Hydroxyvitamin D3, 7α,25-Dihydroxycholesterol, (3S,10R,13R)-17-(5-(dimethylamino)pentan-2-yl)-10,13-dimethyl-2,3,4,7,8,9,10,11,12,13,14,15,16,17-tetradecahydro-1H-cyclopenta[a]phenanthren-3-ol, rel-(3R,10S,13S)-17-[(2S)-6-hydroxy-6-phenylheptan-2-yl]-10,13-dimethyl-2,3,4,7,8,9,10,11,12,13,14,15,16,17-tetradecahydro-1H-cyclopenta[a]phenanthren-3-ol, 27-Nor-25-ketocholesterol and (3alpha,9xi,14xi)-3-hydroxychol-5-en-24-oic acid or a salt thereof.

Embodiment 18: The method according to any one of embodiments 1-17, wherein said agent is selected from a group consisting of 25-hydroxycholesterol (25HC), 24(S)-Hydroxycholesterol (24(S)HC) and 27-Hydroxycholesterol (27HC) or a salt thereof.

Embodiment 19: The method according to any one of embodiments 1-18, wherein said agent is 25-hydroxycholesterol (25HC) or a salt thereof.

Embodiment 20: The method according to any one of embodiments 1-15 wherein said agent comprises a molecular tweezers that inhibits aggregation of a CRYAB protein.

Embodiment 21: The method according to any one of embodiments 1-15 and 20, wherein said agent comprises the CLR01 molecular tweezers.

Embodiment 22: The method according to any one of embodiments 1-15, wherein said agent comprises an inhibitory nucleic acid.

Embodiment 23: The method according to any one of embodiments 1-15 and 22, wherein said agent comprises an inhibitory nucleic acid selected from the group consisting of an interfering RNA molecule, (e.g., shRNA or siRNA), dsRNA, RNA polymerase III transcribed DNA, antisense nucleic acids, and a ribozyme.

Embodiment 24: The method according to any one of embodiments 1-15 and 23, wherein said agent comprises an shRNA or an siRNA that inhibits CRYAB expression.

Embodiment 25: The method according to any one of embodiments 1-15, wherein said agent comprises a CRISPR/Cas system that targets CRYAB, a zinc finger nuclease (ZFN) that targets CRYAB, or a transcription activator-like effector nuclease (TALEN) that targets CRYAB.

Embodiment 26: The method according to any one of embodiments 1-15, wherein said agent comprises an antibody that binds to a CRYAB protein.

Embodiment 27: The method according to any one of embodiments 1-12, wherein said senolytic agent is a small molecule senolytic agent that selectively kills senescent cells in comparison with non-senescent cells.

Embodiment 28: The method of embodiment 27, wherein the senolytic agent is a specific inhibitor of MDM2, Bcl-xL or Akt.

Embodiment 29: The method according to any one of embodiments 27-28, wherein the senolytic agent comprises an inhibitor of Bcl-xL or Bcl-2.

Embodiment 30: The method according to any one of embodiments 27-28, wherein the senolytic agent comprises an inhibitor of MDM2.

Embodiment 31: The method of embodiment 30, wherein the MDM2 inhibitor comprises an imidazoline compound (e.g., a cis-imidazoline compound), a dihydroimidazothiazole compound, a spiro-oxindole compound, a benzodiazepine compound, or a piperidinone.

Embodiment 32: The method of embodiment 30, wherein the MDM2 inhibitor is selected from the group consisting of Nutlin-1, Nutlin-2, RG-7112, RG7388, RO5503781, DS-3032b, MI-63, MI-126, MI-122, MI-142, MI-147, MI-18, MI-219, MI-220, MI-221, MI-773, 3-(4-chlorophenyl)-34(1-(hydroxymethyl)cyclopropyl)methoxy)-2-(4-nitrobenzyl)isoindolin-1-one, Serdemetan, AM-8553, CGM097, RO-2443, and RO-5963.

Embodiment 33: The method of embodiment 31, wherein the senolytic agent comprises an imidazoline compound.

Embodiment 34: The method of embodiment 33, wherein the imidazoline compound comprises a compound having the structure:

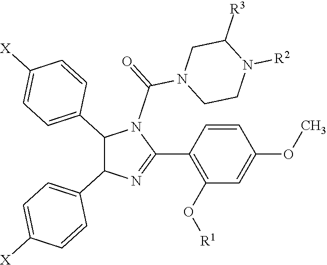

or a pharmaceutically acceptable salt thereof; wherein: X is halide; R¹ is alkyl, R² is —H or heteroalkyl, and R³ is —H or ═O.

Embodiment 35: The method of embodiment 34, wherein the imidazoline compound is selected from the group consisting of nutlin-1, nutlin-2, and nutlin-3.

Embodiment 36: The method of embodiment 34, wherein the imidazoline compound comprises a 4-[[(4S,5R)-4,5-bis(4-chlorophenyl)-4,5-dihydro-2-[4-methoxy-2-(1-methyle-thoxy)phenyl]-1H-imidazol-1-yl]carbonyl]-2-piperazinone or a pharmaceutically acceptable salt thereof.

Embodiment 37: The method of embodiment 33, wherein the imidazoline compound comprises a compound having the structure:

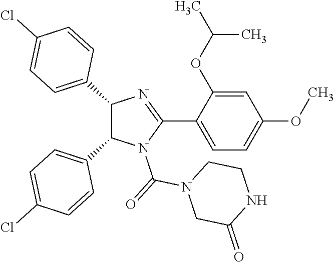

or a pharmaceutically acceptable salt thereof.

Embodiment 38: The method according to any one of embodiments 1-37, wherein said agent is administered systemically.

Embodiment 39: The method according to any one of embodiments 1-37, wherein said agent is administered topically, transdermally, or intradermally.

Embodiment 40: The method according to any one of embodiments 1-37, wherein said agent is administered intranasally, by inhalation, intratracheally, or by intubation.

Embodiment 41: The method according to any one of embodiments 1-40, wherein said subject is a human.

Embodiment 42: The method according to any one of embodiments 1-40, wherein said subject is a non-human mammal.

Embodiment 43: The method according to any one of embodiments 1-42, wherein said subject, when administered said senolytic agent is not diagnosed with and/or under treatment for a pathology associated with aggregation proteins other than CRYAB.

Embodiment 44: The method according to any one of embodiments 1-42, wherein said subject, when administered 25-hydroxycholesterol or a salt thereof, is not diagnosed with and/or under treatment for a pathology associated with aggregation proteins other than CRYAB.

Embodiment 45: The method according to any one of embodiments 1-44, wherein said subject is not diagnosed with and/or under treatment for a pathology characterized by aggregation of a protein selected from the group consisting of Aβ, tau, and alpha-synuclein.

Embodiment 46: The method according to any one of embodiments 1-44, wherein said subject, when administered CLR01, is not diagnosed with and/or under treatment for a pathology characterized by aggregation of a protein selected from the group consisting of Aβ, tau, and alpha-synuclein.

Embodiment 47: The method according to any one of embodiments 1-44, wherein said subject, when administered a senolytic agent, is not diagnosed with and/or under treatment for a pathology characterized by aggregation of a protein selected from the group consisting of Aβ, tau, and alpha-synuclein.

Embodiment 48: The method according to any one of embodiments 1-44, wherein said subject, when administered 25-hydroxycholesterol or a salt thereof, is not diagnosed with and/or under treatment for a pathology characterized by aggregation of a protein selected from the group consisting of Aβ, tau, and alpha-synuclein.

Embodiment 49: The method according to any one of embodiments 1-48, wherein said subject is not under treatment for a neurological pathology.

Embodiment 50: The method according to any one of embodiments 1-48, wherein said subject, when administered CLR01, is not under treatment for a neurological pathology.

Embodiment 51: The method according to any one of embodiments 1-48, wherein said subject, when administered an agent selected from the group consisting of: 25-hydroxycholesterol, 24(S)-Hydroxycholesterol (24(S)HC) and 27-Hydroxycholesterol (27HC) or a salt thereof, is not under treatment for a neurological pathology.

Embodiment 52: The method according to any one of embodiments 1-48, wherein said subject, when administered 25-hydroxycholesterol or a salt thereof, is not under treatment for a neurological pathology.

Embodiment 53: The method according to any one of embodiments 1-52, wherein said subject is not under treatment for a condition selected from the group consisting of Alzheimer's disease and related dementias, amyloid or other cause-mediated mild cognitive impairment (MCI), brain or spinal cord injury (including, but not limited to stroke), Huntingtin's disease, and Parkinson's disease.

Embodiment 54: The method according to any one of embodiments 1-52, wherein said subject, when administered CLR01, is not under treatment for a condition selected from the group consisting of Alzheimer's disease and related dementias, amyloid or other cause-mediated mild cognitive impairment (MCI), brain or spinal cord injury (including, but not limited to stroke), Huntingtin's disease, and Parkinson's disease.

Embodiment 55: The method according to any one of embodiments 1-52, wherein said subject, when administered a senolytic agent, is not under treatment for a condition selected from the group consisting of Alzheimer's disease and related dementias, amyloid or other cause-mediated mild cognitive impairment (MCI), brain or spinal cord injury (including, but not limited to stroke), Huntington's disease, and Parkinson's disease.

Embodiment 56: The method according to any one of embodiments 1-52, wherein said subject, when administered 25-hydroxycholesterol or a salt thereof, is not under treatment for a condition selected from the group consisting of Alzheimer's disease and related dementias, amyloid or other cause-mediated mild cognitive impairment (MCI), brain or spinal cord injury (including, but not limited to stroke), Huntingtin's disease, and Parkinson's disease.

Embodiment 57: The method according to any one of embodiments 1-52, wherein said subject is not under treatment for an ophthalmic disorder.

Embodiment 58: The method according to any one of embodiments 1-52, wherein said subject, when administered CLR01, is not under treatment for an ophthalmic disorder.

Embodiment 59: The method according to any one of embodiments 1-58, wherein said subject, when administered an agent selected from the group consisting of 25-hydroxycholesterol, 24(S)-Hydroxycholesterol (24(S)HC) and 27-Hydroxycholesterol (27HC) or a salt thereof, is not under treatment for an ophthalmic disorder.

Embodiment 60: The method according to any one of embodiments 1-58, wherein said subject, when administered 25-hydroxycholesterol or a salt thereof, is not under treatment for an ophthalmic disorder.

Embodiment 61: A method of selectively killing one or more senescent cells in a subject in need thereof said method comprising: administering to said subject an effective amount of an agent that inhibits expression and/or aggregation of a CRYAB protein.

Embodiment 62: The method of embodiment 61, wherein said agent is selected from the group consisting of a small organic molecule, an inhibitory nucleic acid, an antibody, a CRISPR/Cas system, a zinc finger nuclease (ZFN), and a transcription activator-like effector nuclease (TALEN).

Embodiment 63: The method according to any one of embodiments 61-62, wherein said agent comprises a small organic molecule.

Embodiment 64: The method according to any one of embodiments 61-63, wherein said agent is selected from the group consisting of 25-hyddroxycholesterol (25HC), 24(S)-Hydroxycholesterol (24(S)HC), 27-Hydroxycholesterol (27HC), 22(R)-Hydroxycholesterol (22(R)HC), 7α-Hydroxycholesterol (7αHC), 7β-Hydroxycholesterol (7βHC), Calcifediol 25-Hydroxyvitamin D3, 7α,25-Dihydroxycholesterol, (3S,10R,13R)-17-(5-(dimethylamino)pentan-2-yl)-10,13-dimethyl-2,3,4,7,8,9,10,11,12,13,14,15,16,17-tetradecahydro-1H-cyclopenta[a]phenanthren-3-ol, rel-(3R,10S,13S)-17-[(2S)-6-hydroxy-6-phenylheptan-2-yl]-10,13-dimethyl-2,3,4,7,8,9,10,11,12,13,14,15,16,17-tetradecahydro-1H-cyclopenta[a]phenanthren-3-ol, 27-Nor-25-ketocholesterol and (3alpha,9xi,14xi)-3-hydroxychol-5-en-24-oic acid or a salt thereof.

Embodiment 65: The method according to any one of embodiments 61-64, wherein said agent is selected from a group consisting of 25-hydroxycholesterol (25HC), 24(S)-Hydroxycholesterol (24(S)HC) and 27-Hydroxycholesterol (27HC) or a salt thereof.

Embodiment 66: The method according to any one of embodiments 61-65, wherein said agent is 25-hydroxycholesterol (25HC) or a salt thereof.

Embodiment 67: The method according to any one of embodiments 61-62 wherein said agent comprises a molecular tweezers that inhibits aggregation of a CRYAB protein.

Embodiment 68: The method according to any one of embodiments 61-62 and 67, wherein said agent comprises the CLR01 molecular tweezers.

Embodiment 69: The method according to any one of embodiments 61-62, wherein said agent comprises an inhibitory nucleic acid.

Embodiment 70: The method according to any one of embodiments 61-62 and 69, wherein said agent comprises an inhibitory nucleic acid selected from the group consisting of an interfering RNA molecule, (e.g., shRNA or siRNA), dsRNA, RNA polymerase III transcribed DNA, antisense nucleic acids, and a ribozyme.

Embodiment 71: The method according to any one of embodiments 61-62 and 70, wherein said agent comprises an shRNA or an siRNA that inhibits CRYAB expression.

Embodiment 72: The method according to any one of embodiments 61-62, wherein said agent comprises a CRISPR/Cas system that targets CRYAB, a zinc finger nuclease (ZFN) that targets CRYAB, or a transcription activator-like effector nuclease (TALEN) that targets CRYAB.

Embodiment 73: The method according to any one of embodiments 61-62, wherein said agent comprises an antibody that binds to a CRYAB protein.

Embodiment 74: The method according to any one of embodiments 61-73, wherein said subject has received or is receiving or will receive a DNA damaging or cytotoxic therapy.

Embodiment 75: The method of embodiment 74, wherein said DNA damaging therapy or cytotoxic therapy comprises a treatment for cancer.

Embodiment 76: The method of embodiments 74-75, wherein said DNA damaging or cytotoxic therapy comprises a treatment for a cancer selected from the group consisting of acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, chronic myelogenous leukemia (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

Embodiment 77: The method according to any one of embodiments 74-76, wherein the administration of the agent that inhibits CRYAB expression or aggregation is an adjunct therapy to said treatment for cancer.

Embodiment 78: The method according to any one of embodiments 74-77, wherein said DNA damaging therapy and/or cytotoxic therapy is selected from the group consisting of gamma-irradiation, alkylating agents such as nitrogen mustards (chlorambucil, cyclophosphamide, ifosfamide, melphalan), nitrosoureas (streptozocin, carmustine, lomustine), alkyl sulfonates (busulfan), triazines (dacarbazine, temozolomide) and ethylenimines (thiotepa, altretamine), platinum drugs such as cisplatin, carboplatin, oxalaplatin, antimetabolites such as 5-fluorouracil, 6-mercaptopurine, capecitabine, cladribine, clofarabine, cytarabine, floxuridine, fludarabine, gemcitabine, hydroxyurea, methotrexate, pemetrexed, pentostatin, thioguanine, anthracyclines such as daunorubicin, doxorubicin, epirubicin, idarubicin, anti-tumor antibiotics such as actinomycin-D, bleomycin, mitomycin-C, mitoxantrone, topoisomerase inhibitors such as topoisomerase I inhibitors (topotecan, irinotecan) and topoisomerase II inhibitors (etoposide, teniposide, mitoxantrone), mitotic inhibitors such as taxanes (paclitaxel, docetaxel), epothilones (ixabepilone), vinca alkaloids (vinblastine, vincristine, vinorelbine), estramustine, cyclin-dependent kinase inhibitors (roscovitine, palbociclib, abemaciclib, olaparib), epigenetic modifiers (curcumin, valproic acid), and HIV medications such as NRTIs (Nucleoside Reverse Transcriptase Inhibitors), NNRTIs (Non-Nucleoside Reverse Transcriptase Inhibitors), and protease inhibitors (azidothymidine, tenofovir, emtricitabine, abacavir, nevirapine, atazanavir, lopinavir).

Embodiment 79: The method according to any one of embodiments 61-78, wherein said method delays the onset and/or slow or stops the progression of one or more symptoms associated with accumulation of senescent cells from said DNA damaging therapy.

Embodiment 80: The method according to any one of embodiments 61-79, wherein said method delays the onset and/or slow or stops the progression of one or more features of aging in the subject.

Embodiment 81: The method of embodiment 80, wherein said feature of aging is selected from the group consisting of: systemic decline of the immune system, muscle atrophy and decreased muscle strength, decreased skin elasticity, delayed wound healing, retinal atrophy, reduced lens transparency, reduced hearing, osteoporosis, sarcopenia, hair graying, skin wrinkling, poor vision, frailty, cognitive impairment, ophthalmic disease, and idiopathic pulmonary fibrosis.

Embodiment 82: The method according to any one of embodiments 61-81, wherein said method reduces the severity and/or ameliorates one or more symptoms and/or delays the onset and/or slows or stops the progression of a senescence-associated disease or disorder.

Embodiment 83: The method of embodiment 82, wherein the senescence-associated disease or disorder is selected from the group consisting of cardiovascular disease, Alzheimer's disease and related dementias, Parkinson's disease, cataracts, macular degeneration, glaucoma, atherosclerosis, acute coronary syndrome, myocardial infarction, stroke, hypertension, idiopathic pulmonary fibrosis (IPF), chronic obstructive pulmonary disease (COPD), osteoarthritis, type 2 diabetes, obesity, fat dysfunction, coronary artery disease, cerebrovascular disease, periodontal disease, and cancer treatment-related disability such as atrophy and fibrosis in various tissues, brain and heart injury, and therapy-related myelodysplastic syndromes. Additionally, an age-related pathology may include an accelerated aging disease such as progeroid syndromes (i.e. Hutchinson-Gilford progeria syndrome, Werner syndrome, Bloom syndrome, Rothmund-Thomson Syndrome, Cockayne syndrome, xeroderma pigmentosum, trichothiodystrophy, combined xeroderma pigmentosum-Cockayne syndrome, restrictive dermopathy), ataxia telangiectasia, Fanconi anemia, Friedreich's ataxia, dyskeratosis congenital, aplastic anemia, IPF, renal dysfunction, kyphosis, herniated intervertebral disc, frailty, hair loss, hearing loss, vision loss (blindness or impaired vision), muscle fatigue, skin conditions, skin nevi, diabetes, metabolic syndrome, sarcopenia, dermatological conditions (e.g., wrinkles, including superficial fine wrinkles; hyperpigmentation; scars; keloid; dermatitis; psoriasis; eczema (including seborrheic eczema); rosacea; vitiligo; ichthyosis vulgaris; dermatomyositis; and actinic keratosis).

Embodiment 84: The method of embodiment 82, wherein the senescence-associated disease or disorder is a cardiovascular disease selected from the group consisting of atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke.

Embodiment 85: The method of embodiment 84, wherein the senescence-associated disease comprises a cardiovascular disease.

Embodiment 86: The method of embodiment 85, wherein said method comprises ameliorating a symptom selected from the group consisting of irregularity in heart rhythm, age-related cellular hypertrophy, increase in the cross-sectional area of a cardiomyocyte and decrease in cardiac stress tolerance.

Embodiment 87: The method of embodiment 82, wherein the senescence-associated disease comprises osteoarthritis.

Embodiment 88: The method of embodiment 82, wherein the senescence-associated disease comprises atherosclerosis.

Embodiment 89: The method of embodiment 82, wherein the senescence-associated disease comprises a pulmonary disease.

Embodiment 90: The method of embodiment 89, wherein said pulmonary disease is selected from the group consisting of pulmonary fibrosis, chronic obstructive pulmonary disease, asthma, cystic fibrosis, emphysema, bronchiectasis, and age-related loss of pulmonary function.

Embodiment 91: The method of embodiment 82, wherein the senescence-associated disease or disorder is an inflammatory or autoimmune disease or disorder selected from the group consisting of osteoarthritis, osteoporosis, oral mucositis, inflammatory bowel disease, kyphosis, and herniated intervertebral disc.

Embodiment 92: The method of embodiment 82, wherein the senescence-associated disease or disorder is a neurodegenerative disease selected from the group consisting of Alzheimer's disease, Parkinson's disease, Huntington's disease, dementia, mild cognitive impairment, and motor neuron dysfunction.

Embodiment 93: The method of embodiment 82, wherein the senescence-associated disease or disorder comprises a metabolic disease selected from the group consisting of diabetes, diabetic ulcer, metabolic syndrome, and obesity.

Embodiment 94: The method of embodiment 82, wherein the senescence-associated disease comprises an eye disease or disorder selected from the group consisting of macular degeneration, glaucoma, cataracts, presbyopia, and vision loss.

Embodiment 95: The method of embodiment 82, wherein the senescence-associated disease comprises an age-related disorder selected from the group consisting of renal disease, renal failure, frailty, hearing loss, muscle fatigue, skin conditions, skin wound healing, liver fibrosis, pancreatic fibrosis, oral submucosa fibrosis, and sarcopenia.

Embodiment 96: The method of embodiment 82, wherein the senescence-associated disease comprises a dermatological disease or disorder selected from the group consisting of eczema, psoriasis, hyperpigmentation, nevi, rashes, atopic dermatitis, urticaria, diseases and disorders related to photosensitivity or photoaging, rhytides; pruritis; dysesthesia; eczematous eruptions; eosinophilic dermatosis; reactive neutrophilic dermatosis; pemphigus; pemphigoid; immunobullous dermatosis; fibrohistocytic proliferations of skin; cutaneous lymphomas; and cutaneous lupus.

Embodiment 97: The method according to any one of embodiments 61-96, wherein said agent is administered directly to an organ or tissue that comprises the senescent cells.

Embodiment 98: The method according to any one of embodiments 61-96, wherein said agent is administered systemically.

Embodiment 99: The method according to any one of embodiments 61-96, wherein said agent is administered topically, transdermally, or intradermally.

Embodiment 100: The method according to any one of embodiments 61-96, wherein said agent is administered intranasally, by inhalation, intratracheally, or by intubation.

Embodiment 101: The method according to any one of embodiments 61-100, wherein said subject is a human.

Embodiment 102: The method according to any one of embodiments 61-100, wherein said subject is a non-human mammal.

Embodiment 103: The method according to any one of embodiments 61-73, wherein said subject a pathology characterized by the generation of senescent cells and an inflammatory response.

Embodiment 104: The method of embodiment 103, wherein said pathology comprises kyphosis and/or herniated intervertebral discs, and/or osteoporosis.

Embodiment 105: The method of embodiment 103, wherein said pathology comprises irritable bowel syndrome and/or an inflammatory bowel disease.

Embodiment 106: The method of embodiment 105, wherein said pathology comprises colitis and/or Crohn's disease.

Embodiment 107: The method of embodiment 103, wherein said pathology comprises a pulmonary disease.

Embodiment 108: The method of embodiment 107, wherein said pathology comprise a pathology selected from the group consisting of idiopathic pulmonary fibrosis (IPF), chronic obstructive pulmonary disease (COPD), asthma, cystic fibrosis, bronchiectasis, and emphysema.

Embodiment 109: The method of embodiment 103, wherein said pathology comprises a pathology characterized by fibrosis.

Embodiment 110: The method of embodiment 109, wherein said pathology comprises a pathology selected from the group consisting of renal fibrosis, liver fibrosis, pancreatic fibrosis, cardiac fibrosis, skin wound healing, and oral submucous fibrosis.

Embodiment 111: The method according to any one of embodiments 61-110, wherein said subject is not diagnosed with and/or under treatment for a pathology associated with aggregation proteins other than CRYAB.

Embodiment 112: The method according to any one of embodiments 61-110, wherein said subject, when administered CLR01, is not diagnosed with and/or under treatment for a pathology associated with aggregation proteins other than CRYAB.

Embodiment 113: The method according to any one of embodiments 61-110, wherein said subject, when administered agent which is selected from a group consisting of 25-hydroxycholesterol, 24(S)-Hydroxycholesterol (24(S)HC) and 27-Hydroxycholesterol (27HC) or a salt thereof, is not diagnosed with and/or under treatment for a pathology associated with aggregation proteins other than CRYAB.

Embodiment 114: The method according to any one of embodiments 61-110, wherein said subject, when administered 25-hydroxycholesterol or a salt thereof, is not diagnosed with and/or under treatment for a pathology associated with aggregation proteins other than CRYAB.

Embodiment 115: The method according to any one of embodiments 61-114, wherein said subject is not diagnosed with and/or under treatment for a pathology characterized by aggregation of a protein selected from the group consisting of Aβ, tau, and alpha-synuclein.

Embodiment 116: The method according to any one of embodiments 61-114, wherein said subject, when administered CLR01, is not diagnosed with and/or under treatment for a pathology characterized by aggregation of a protein selected from the group consisting of Aβ, tau, and alpha-synuclein.

Embodiment 117: The method according to any one of embodiments 61-114, wherein said subject, when administered an agent selected from the group consisting of: 25-hydroxycholesterol, 24(S)-Hydroxycholesterol (24(S)HC) and 27-Hydroxycholesterol (27HC) or a salt thereof, is not diagnosed with and/or under treatment for a pathology characterized by aggregation of a protein selected from the group consisting of Aβ, tau, and alpha-synuclein.

Embodiment 118: The method according to any one of embodiments 61-114, wherein said subject, when administered 25-hydroxycholesterol or a salt thereof, is not diagnosed with and/or under treatment for a pathology characterized by aggregation of a protein selected from the group consisting of Aβ, tau, and alpha-synuclein.

Embodiment 119: The method according to any one of embodiments 61-117, wherein said subject is not under treatment for a neurological pathology.

Embodiment 120: The method according to any one of embodiments 61-117, wherein said subject, when administered CLR01, is not under treatment for a neurological pathology.

Embodiment 121: The method according to any one of embodiments 61-117, wherein said subject, when administered an agent selected from the group consisting of: 25-hydroxycholesterol, 24(S)-Hydroxycholesterol (24(S)HC) and 27-Hydroxycholesterol (27HC) or a salt thereof, is not under treatment for a neurological pathology.

Embodiment 122: The method according to any one of embodiments 61-117, wherein said subject, when administered 25-hydroxycholesterol or a salt thereof, is not under treatment for a neurological pathology.

Embodiment 123: The method according to any one of embodiments 61-122, wherein said subject is not under treatment for a condition selected from the group consisting of Alzheimer's disease and related dementias, amyloid or other cause-mediated mild cognitive impairment (MCI), brain or spinal cord injury (including, but not limited to stroke), Huntingtin's disease, and Parkinson's disease.

Embodiment 124: The method according to any one of embodiments 61-122, wherein said subject, when administered CLR01, is not under treatment for a condition selected from the group consisting of Alzheimer's disease and related dementias, amyloid or other cause-mediated mild cognitive impairment (MCI), brain or spinal cord injury (including, but not limited to stroke), Huntingtin's disease, and Parkinson's disease.

Embodiment 125: The method according to any one of embodiments 61-122, wherein said subject, when administered and agent selected from the group consisting of: 25-hydroxycholesterol, 24(S)-Hydroxycholesterol (24(S)HC) and 27-Hydroxycholesterol (27HC) or a salt thereof, is not under treatment for a condition selected from the group consisting of Alzheimer's disease and related dementias, amyloid or other cause-mediated mild cognitive impairment (MCI), brain or spinal cord injury (including, but not limited to stroke), Huntington's disease, and Parkinson's disease.

Embodiment 126: The method according to any one of embodiments 61-122, wherein said subject, when administered 25-hydroxycholesterol or a salt thereof, is not under treatment for a condition selected from the group consisting of Alzheimer's disease and related dementias, amyloid or other cause-mediated mild cognitive impairment (MCI), brain or spinal cord injury (including, but not limited to stroke), Huntingtin's disease, and Parkinson's disease.

Embodiment 127: The method according to any one of embodiments 61-126, wherein said subject is not under treatment for an ophthalmic disorder.

Embodiment 128: The method according to any one of embodiments 61-126, wherein said subject, when administered CLR01, is not under treatment for an ophthalmic disorder.

Embodiment 129: The method according to any one of embodiments 61-126, wherein said subject, when administered an agent selected from the group consisting of 25-hydroxycholesterol, 24(S)-Hydroxycholesterol (24(S)HC) and 27-Hydroxycholesterol (27HC) or a salt thereof, is not under treatment for an ophthalmic disorder.

Embodiment 130: The method according to any one of embodiments 61-129, wherein said subject, when administered 25-hydroxycholesterol or a salt thereof, is not under treatment for an ophthalmic disorder.

### Definitions

A senolytic agent (e.g., CRYAB inhibitor) as used herein is an agent that “selectively” (preferentially or to a greater degree) destroys, kills, removes, or facilitates selective destruction of senescent cells. In other words, the senolytic agent destroys or kills senescent cells in a biologically, clinically, and/or statistically significant manner compared with its capability to destroy or kill non-senescent cells. A senolytic agent is used in an amount and for a time sufficient to selectively kill established senescent cells but is insufficient to kill (destroy, cause the death of) non-senescent cells in a clinically significant or biologically significant manner. In certain embodiments, the senolytic agents described herein alter at least one signaling pathway in a manner that induces (initiates, stimulates, triggers, activates, promotes) and results in (i.e., causes, leads to) death of the senescent cells. The senolytic agent may alter, for example, either or both of a cell survival signaling pathway (e.g., Akt pathway) or an inflammatory pathway, for example, by antagonizing a protein within the cell survival and/or inflammatory pathway in a senescent cell.

The term “CRYAB protein” refers to a protein expressed by a crystallin alpha B (CRYAB) gene.

The term “CRYAB” inhibitor refers to an agent that inhibits the expression and/or aggregation of a CRYAB protein. In certain embodiments, the “CRYAB inhibitor” inhibits transcription and/or translation (e.g., expression) of the CRYAB gene.

When used in the context of the methods provided herein, the term “one or more senolytic agents” refers to the use of a CRYAB inhibitor as described herein (e.g., 25HC, CLR01, T3MG/NCI-41356 etc.) or to the use of a CRYAB inhibitor as described herein in combination with one or more additional senolytic agents. In certain embodiments, the additional senolytic agent(s) comprises a second CRYAB inhibitor, and/or other senolytic agents including, but not limited to those described in U.S. Patent Publication Nos: US 2019/0022090, US 2019/0000846, US 2018/0303828, US 2018/0256568, US 2018/0235957, US 2018/0235956, US 2018/0193458, US 2018/0117038, US 2017/0348307, US 2017/0326136, US 2017/0224680, US 2017/0209435, US 2017/0198253, US 2017/0196858, US 2017/0196857, US 2016/0339019, US 2016/0038576, and the like. In certain illustrative embodiments, the additional senolytic agents can include an MDM2 inhibitor (e.g., Nutlin-3a, Nutlin-3b, RG-7112, RG7388, RO5503781, MI-63, MI-126, MI-122, MI-142, MI-147, MI-18, MI-219, MI-220, MI-221, MI-773, 3-(4-chlorophenyl)-3-((1-(hydroxymethyl)cyclopropyl)methoxy)-2-(4-nitrobe-nzyl)isoindolin-1-one, RO-2443, RO-5963, AM-8553, WEHI-539, A-1155463, A-1331852, ABT-263, ABT-199, ABT-737, MK-2206, CCT128930, JNK-IN-8, sanguinarine chloride, methyl 3-(4-nitrophenyl) propiolate (NPP), AT7867, AZD7762, sunitinib, GDC-0980, BKM120, NQDI-1, R406, erlotinib, CYM 7008-00-01, GlcNAc, olaparib, AMG-232, NVP-CGM097, MI-773, CAY10681, CAY10682, Y239-EE, RG-7112, a Boronate, RO-5963, HLI 373, JNJ 26854165, MEL23 MI-773, RG-7112, JNJ 26854165, AD20187, and the like), and/or an inhibitor of one or more BCL-2 anti-apoptotic protein family members wherein the inhibitor inhibits at least BCL-xL BCL2 (e.g., ABT-263, ABT-737, WEHI-539, A-1155463, a benzothiazole-hydrazone compound (e.g., WEHI-539), an aminopyridine compound, a benzimidazole compound, a tetrahydroquinolin compound, a phenoxyl compound, and the like) and/or an Akt-specific inhibitor (e.g., MK-2206).

The terms “subject,” “individual,” and “patient” may be used interchangeably and refer to humans, as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that is characterized by elevated levels of senescent cells and/or a pathology characterized by elevated levels of senescent cells, and/or undergoing a treatment known to elevate levels of senescent cells.

The term “treat” when used with reference to treating, e.g., a pathology or disease refers to the mitigation and/or elimination of one or more symptoms of that pathology or disease, and/or a delay in the progression and/or a reduction in the rate of onset or severity of one or more symptoms of that pathology or disease, and/or the prevention of that pathology or disease. The term _“treat”_can refer to prophylactic treatment, which includes a delay in the onset or the prevention of the onset of a pathology or disease.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10): 1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110: 4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments. In addition, it is possible that nucleic acids of the present invention can alternatively be triple-stranded.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′2, a dimer of Fab which itself is a light chain joined to V_{H}-C_{H}1 by a disulfide bond. The F(ab)′2 may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H}-V_{L} heterodimer, which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fv's (scFv), however, alternative expression strategies have also been successful. For example Fab molecules can be displayed on phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No. 5,733,743). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778). Particularly preferred antibodies should include all that have been displayed on phage (e.g., scFv, Fv, Fab and disulfide linked Fv (Reiter et al. (1995) Protein Eng. 8: 1323-1331). In certain embodiments, antibodies also include peptibodies. Peptibodies consist of biologically active peptides grafted onto an Fc domain. This approach retains certain desirable features of antibodies, notably an increased apparent affinity through the avidity conferred by the dimerization of two Fcs and a long plasma residency time (see, e.g., Shimamoto et al. (2012) Mabs, 4(5): 586-591).

The term “biological sample” refers to a sample that is a sample of biological tissue, cells, or fluid that, in a healthy and/or pathological state, contains one or more of the indicators of senescent cells described herein. Such samples include, but are not limited to, cultured cells, acute cell preparations, sputum, amniotic fluid, blood, blood cells (e.g., white cells), tissue or fine needle biopsy samples, urine, peritoneal fluid, and pleural fluid, or cells therefrom. Biological samples may also include sections of tissues such as frozen sections taken for histological purposes. Although the sample is typically taken from a human patient, the assays can be used on samples from any mammal, such as dogs, cats, sheep, cattle, rodents (mice, rats, etc.), lagomorphs (rabbits, hares, etc.) and pigs, etc. The sample may be pretreated as necessary by dilution in an appropriate buffer solution or concentrated, if desired. Any of a number of standard aqueous buffer solutions, employing one of a variety of buffers, such as phosphate, Tris, or the like, at physiological pH can be used.

The term “small organic molecule” refers to a molecule of a size comparable to those organic molecules generally used in pharmaceuticals. The term excludes biological macromolecules (e.g., proteins, nucleic acids, etc.). In certain embodiments, preferred small organic molecules range in size up to about 5000 Da, or up to about 4000 Da, or up to about 3,000 Da, or up to about 2000 Da, or up to about 1000 Da.

### BRIEF DESCRIPTION OF THE DRAWINGS

FIG. 1 shows that doxorubicin (Doxo) induces senescence marker, p16INK4a, in mouse dermal fibroblasts (mDFs). As shown, mDFs undergo senescence upon 250 nM of Doxo treatment for 24 hours (Day 0). Real-time quantitative PCR (qPCR) was performed on RNA samples isolated from DMSO treated NS (Non-Senescent) mDF cells or Doxo treated SEN (Senescent) mDF cells at day 1, 5, and 14 after Doxo treatment. The amount of mRNA was normalized to the level of β-actin (see, e.g., Demaria et al. (2017) Cancer Discovery, 2: 165-176; and references cited therein). Relative levels of p16INK4a mRNA for DMSO and Doxo treated mDFs was measured. The average value of DMSO is expressed as 1. (Mean +/−SE, n=4, **p<0.01, ****p<0.0001 by Dunnett's multiple comparisons test vs DMSO).

FIG. 2 shows that senescent mDFs upregulated CRYAB. Real-time quantitative PCR (qPCR) was performed on RNA samples isolated from DMSO treated NS mDF cells or Doxo treated SEN mDF cells at day 1, 5, and 14 after Doxo treatment. The amount of mRNA was normalized to the level of β-actin (see, e.g., Demaria et al. (2017) Cancer Discovery, 2: 165-176; and references cited therein). Relative levels of CRYAB mRNA for DMSO and Doxo treated mDFs was measured. The average value of DMSO is expressed as 1. (Mean +/−SE, n=4, *p<0.05, ***p<0.001 by Dunnett's multiple comparisons test vs DMSO).

FIG. 3 shows that the Doxo induced senescence in mouse primary fibro adipogenic progenitors (FAPs). As shown, FAPs undergo senescence upon 250 nM of Doxo treatment for 24 hours (Day 0). The mRNA was extracted from DMSO treated NS or Doxo treated SEN FAPs at indicated time points and quantified by qPCR. The amount of mRNA was normalized to the level of β-actin. The graphs show the relative levels of p16INK4a mRNA for DMSO and Doxo treated samples. The average value of DMSO is expressed as 1 (Mean +/−SE, n=3, *p<0.05 by Dunnett's multiple comparisons test vs DMSO).

FIG. 4 shows that the expression of CRYAB mRNA was significantly increased after induction of senescence in FAPs. The cells were treated with 250 nM doxorubicin and incubated for 24 hrs (Day 0). The mRNA was extracted from DMSO treated NS or Doxo treated SEN FAPs at the indicated time points and quantified by qPCR. The amount of mRNA was normalized to the level of β-actin mRNA. The average value of DMSO is expressed as 1 (Mean +/−SE, n=3, **p<0.01 and ***p<0.001 by Dunnett's multiple comparisons test vs DMSO).

FIG. 5 shows that the suppressed expression of CRYAB significantly decreased the cell viability of senescent FAPs. The cells were infected with 5 MOI of lentivirus containing scrambled control or CRYAB targeting shRNA sequences from Sigma-Aldrich. After 3 days of puromycin selection (2 ug/ml), the cells were treated with 250 nM doxorubicin and incubated for 24 hrs (Day 0). Cell viability was analyzed by Cell Counting Kit-8 (Dojindo Molecular Technologies Inc.) according to the manufactures' protocol. The value was normalized to that of Day 0 (Mean +/−SE, n=3, *p<0.05, **p<0.01 and ***p<0.001 by Dunnett's multiple comparisons test vs Ctrl/TRC1).

FIG. 6 shows that the suppressed expression of CRYAB significantly increased cytotoxity of senescent FAPs. The cells were infected with 5 MOI of lentivirus containing scrambled control and CRYAB targeting shRNA sequences from Sigma-Aldrich. After 3 days of puromycin selection (2 ug/ml), the cells were treated with 250 nM doxorubicin and incubated for 24 hrs (Day 0). Cytotoxity was analyzed by Cytotoxity LDH Assay Kit-WST 8 (Dojindo Molecular Technologies Inc.) according to the manufactures' protocol. The value was normalized to that of Ctrl/TRC1 (Mean +/−SE, n=3, **p<0.01 and ***p<0.001 by Dunnett's multiple comparisons test vs Ctrl/TRC1).

FIG. 7 shows that the shRNAs for CRYAB drastically suppressed CRYAB expression in FAPs. The cells were infected with 5 MOI of lentivirus containing scrambled control and CRYAB targeting shRNA sequences from Sigma-Aldrich. After 3 days of puromycin selection (2 ug/ml), the cells were treated with 250 nM doxorubicin and incubated for 24 hrs (Day 0). The mRNA was extracted at Day 7 and quantified by qPCR. The amount of mRNA was normalized to that of β-actin. The average value of Ctrl/TRC1 is expressed as 1 (Mean +/−SE, n=3, ***p<0.001 by Dunnett's multiple comparisons test vs Ctrl/TRC1).

FIG. 8 shows that the suppressed expression of CRYAB significantly decreased Timp2 (a natural inhibitor of matrix metalloproteinase that is a marker for SASP) expression in senescent FAPs. The cells were infected with 5 MOI of lentivirus containing scrambled control and CRYAB targeting shRNA sequences from Sigma-Aldrich. After 3 days of puromycin selection (2 ug/ml), the cells were treated with 250 nM doxorubicin and incubated for 24 hrs (Day 0). The mRNA was extracted at Day 7 and quantified by qPCR. The amount of mRNA was normalized by that of β-actin. The average value of Ctrl/TRC1 is expressed as 1 (Mean +/−SE, n=3, ***p<0.001 by Dunnett's multiple comparisons test vs Ctrl/TRC1).

FIG. 9 shows that the suppressed expression of CRYAB didn't decrease Timp2 expression in non-senescent FAPs. The cells were infected with 5 MOI of lentivirus containing scrambled control and CRYAB targeting shRNA sequences from Sigma-Aldrich. After 3 days of puromycin selection (2 ug/ml), the mRNA was extracted and quantified by qPCR. The amount of mRNA was normalized by that of β-actin. The average value of Ctrl/TRC1 is expressed as 1 (Mean +/−SE, n=3, **p<0.01 by Dunnett's multiple comparisons test vs Ctrl/TRC1).

FIG. 10, panels A-B, shows that 25-Hydroxycholesterol (25HC) treatment specifically kills senescent FAPs. Panel A) Timeline for DMSO/Doxo treatment and early-time point timeline for 25HC treatment on NS and SEN cells. 25HC treatment was started immediately after removing DMSO/Doxo. Panel B) Cell viability assay was performed on NS and SEN cells treated with different concentrations of 25HC. 25HC treatment was done for total 5 days. The value was normalized to that of 0 uM. The average value of 0 M is expressed as 100% (Mean +/−SE, n=3, **p<0.01 and ***p<0.001 by student's t-test vs NS).

FIG. 11, panels A-B, shows that 25-Hydroxycholesterol (25HC) treatment specifically kills senescent mDFs at an early-time point after senescence induction. Panel A) Timeline for DMSO/Doxo treatment and early-time point timeline for 25HC treatment on NS and SEN cells. 25HC treatment was started immediately after removing DMSO/Doxo. Panel B) Cell viability assay was performed on NS and SEN cells treated with different concentrations of 25HC. 25HC treatment was done for 72 hrs. (Mean +/−SD, n=3, *p<0.05, **p<0.01 vs 0 uM by student's t-test).

FIG. 12, panels A-B, shows that 25HC treatment specifically kills senescent mDFs at late-time point after senescence induction. Panel A) Timeline for DMSO/Doxo treatment and late-time point timeline for 25HC treatment on NS and SEN cells. 25HC treatment was started 7 days after removing DMSO/Doxo. Panel B) Cell viability assay was performed on NS and SEN cells treated with different concentrations of 25HC. 25HC treatment was done for 72 hrs. (Mean +/−SD, n=6, **p<0.01, ***p<0.001, ****p<0.0001, vs 0 uM by student's t-test).

FIG. 13, panels A-C, shows that treatments with 24(S)-Hydroxycholesterol (24(S)HC) and 27-Hydroxycholesterol (27HC) specifically kill senescent human fetal lung fibroblast (IMR-90; CCL-186, ATCC) at early-time points after senescence induction. Panel A) Timeline for DMSO/250 nM Doxo treatment and early-time point timeline for 24(S)-HC and 27HC treatment on NS and SEN cells. 24(S)HC and 27HC treatment were started immediately after removing DMSO/Doxo. Panel B) and C) Cell viability assay were performed on NS and SEN cells treated with different concentrations of 24(S)HC and 27HC, respectively. 24(S)HC and 27HC treatment were done for 72 hrs. The cell viability was analyzed by Cell Counting Kit-8 according to the manufactures' protocol using Spectra Max 190 (Molecular Devices). The value was normalized to that of DMSO. (Mean +/−SE, n=3, *p<0.05, **p<0.01 and ***p<0.001 by student's t-test).

FIG. 14 shows that senolysis via 25HC and GCV reduces PWV in aged female C57BL/6J p16-3MR mice. Control—untreated f.

### DETAILED DESCRIPTION

In various embodiments, methods and compositions are provided for selectively killing one or more senescent cells in a subject in need thereof. The methods exploit the identification of the crystallin alpha B (CRYAB) gene as a target for senolytic agents.

To identify genes uniquely associated with senescence in skeletal muscle, a two phase strategy was used; 1) focus on specific cell populations purified from skeletal muscle, satellite cells, and fibro adipogenic progenitors (FAPs), and 2) identify genes uniquely associated with either senescence, or the SASP in small populations of cells. Single cell profiling of these cells was used to identify genes associated with senescence. Genetic techniques were then used to inactivate candidate genes associated with senescence and cell survival in response to doxorubicin (DOXO)-induced senescence, and specific senescent cell killing (“senolysis”) via down regulating the target genes was evaluated using conventional molecular techniques.

One such gene identified using this approach was the crystallin alpha B (CRYAB) gene, which appears to be specifically induced by senescence. Genetic knockdown of this gene kills senescent cells. The commercially available compound 25-hydroxycholesterol (25HC) was identified as an effective inhibitor of CRYAB (Makley et al. (2015) Science, 350: 674-7). Testing this compound in the context of cell culture and specifically killing senescent cells demonstrated that 25HC was a novel senolytic agent.

There are some commercially available analogs of 25HC such as oxysterols (Griffiths et al. (2019) Biochem Soc Trans., 47: 517-526). The senolytic effects of several oxysterols were evaluated, and then it was demonstrated that 24(S)-Hydroxycholesterol (24(S)HC; SML1648, Sigma), 27-Hydroxycholesterol (27HC; SML2042, Sigma), 22(R)-Hydroxycholesterol (22(R)HC; H9384, Sigma), 7α-Hydroxycholesterol (7αHC; 700034P, Merck) and 7β-Hydroxycholesterol (7βHC; H6891, Sigma) were novel senolytics. Other 25HC analogs were also evaluated, and it was demonstrated that Calcifediol 25-Hydroxyvitamin D3 (030-14871, Wako), 7α,25-Dihydroxycholesterol (C4241, ApexBio), (3S,10R,13R)-17-(5-(dimethylamino)pentan-2-yl)-10,13-dimethyl-2,3,4,7,8,9,10,11,12,13,14,15,16,17-tetradecahydro-1H-cyclopenta[a]phenanthren-3-ol (FMC Crop Protection), rel-(3R,10S,13S)-17-[(2S)-6-hydroxy-6-phenylheptan-2-yl]-10,13-dimethyl-2,3,4,7,8,9,10,11,12,13,14,15,16,17-tetradecahydro-1H-cyclopenta[a]phenanthren-3-ol (IBS), 27-Nor-25-ketocholesterol (IBS) and (3alpha,9xi,14xi)-3-hydroxychol-5-en-24-oic acid (IBS) were novel senolytics.

Accordingly, in certain embodiments, methods of selectively killing one or more senescent cells in a subject in need thereof are provided wherein the methods involve administering to the subject an effective amount of an agent that inhibits expression and/or aggregation of a CRYAB protein. The methods are expected to selectively diminish or deplete senescent cells, particularly in one or more target organs of interest.

The methods find utility in ameliorating one or more symptoms of senescence-associated and/or age-related diseases and/or slowing the onset and/or progression of senescence-associated and/or age-related diseases. In certain embodiments, the methods find utility in the prevention or treatment of therapy induced senescent cells as described herein. In various embodiments, these methods involve administration an effective amount (dose) of a senolytic agent (e.g., an inhibitor of CRYAB expression and/or aggregation (a.k.a. a CRYAB inhibitor)) as described herein.

### Inhibitors of CRYAB Expression or Aggregation.

In various embodiments, the methods described herein involve administration to a subject one or more agents that inhibit expression and/or aggregation of CRYAB protein (protein expressed by CRYAB gene). Such inhibitors include, but are not limited to small molecule inhibitors, nucleic acid-based CRYAB inhibitors, anti-CRYAB antibodies, CRISPR constructs, zinc finger nucleases (ZFNs), transcription activator-like effector nucleases (TALENs), as well as methods that involve the use of any other nucleases that can cause DNA breaks or bind to DNA.

### Small Molecule Inhibitors

In certain embodiments, the methods described herein utilize various small molecule inhibitors of CRYAB expression and/or protein aggregation. Small molecules are a diverse group of synthetic and natural substances generally having low molecular weights (preferably less than about 2000 Daltons, less than about 1000 Daltons, or less than about 500 Daltons). Small molecules, without limitation, may be, for example, nucleic acids, peptides, polypeptides, peptide nucleic acids, peptidomimetics, carbohydrates, lipids, or other organic (carbon containing) or inorganic molecules and may be synthetic or naturally occurring or optionally derivatized. Such small molecules may be a therapeutically deliverable substance or may be further derivatized to facilitate delivery or targeting.

As described in Example 1, 25-hydroxycholersterol (25HC) (a.k.a. Cholest-5-ene-30,25-diol), shown below, is an effective inhibitor of CRYAB protein aggregation and appears to be an effective senolytic compound that preferentially eliminates senescent cells.

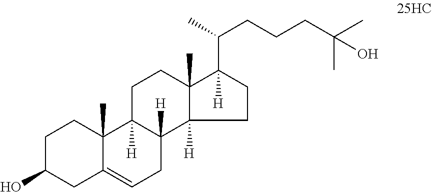

As also described in Example 1, 24(S)-Hydroxycholesterol (a.k.a. 5-Cholesten-3β,24(S)-diol, Cerebrosterin, Cerebrosterol, Cholest-5-ene-3β,24-diol), 27-Hydroxycholesterol (a.k.a. (25R)-26-Hydroxycholesterol, (25R)-Cholest-5-ene-3b,26-diol, 25(R)-27-hydroxy Cholesterol), shown below, is an effective inhibitor of CRYAB protein aggregation and appears to be an effective senolytic compound that preferentially eliminates senescent cells.

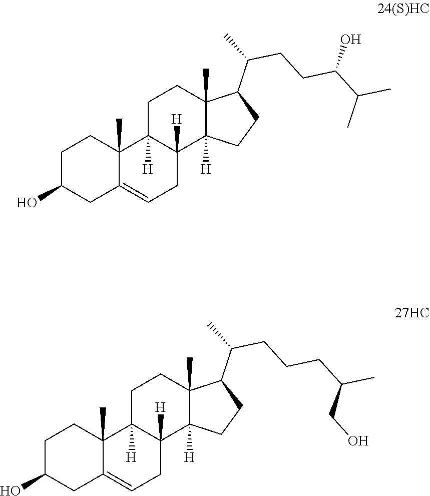

In addition to 25HC, several oxysterols including 22(R)-Hydroxycholesterol (a.k.a. 5-Cholestene-30,22(R)-diol, 5-Cholestene-3β,22[R]-diol), 7α-Hydroxycholesterol (a.k.a. cholest-5-en-3β,7α-diol) and 70-Hydroxycholesterol (a.k.a. 5-Cholestene-30,70-diol) are effective inhibitors of CRYAB protein aggregation and appear to be effective senolytic compounds that preferentially eliminate senescent cells as well as 25HC. The other 25HC analogs such as Calcifediol 25-Hydroxyvitamin D3, 7α,25-Dihydroxycholesterol, (3S,10R,13R)-17-(5-(dimethylamino)pentan-2-yl)-10,13-dimethyl-2,3,4,7,8,9,10,11,12,13,14,15,16,17-tetradecahydro-1H-cyclopenta[a]phenanthren-3-ol, rel-(3R,10S,13S)-17-[(2S)-6-hydroxy-6-phenylheptan-2-yl]-10,13-dimethyl-2,3,4,7,8,9,10,11,12,13,14,15,16,17-tetradecahydro-1H-cyclopenta[a]phenanthren-3-ol, 27-Nor-25-ketocholesterol and (3alpha,9xi,14xi)-3-hydroxychol-5-en-24-oic acid are also effective inhibitors of CRYAB protein aggregation and appear to be effective senolytic compounds that preferentially eliminate senescent cells.

Other small molecule inhibitors of protein aggregation include, but are not limited to molecular tweezers, e.g., as described in U.S. Pat. No. 8,791,092. These molecular tweezers have been shown to effectively inhibit aggregation of CRYAB protein (see, e.g., Xu et al. (2017) J. Am. Heart Assoc. 6: e006182). One illustrative, but non-limiting molecular tweezers is CLR01, shown below.

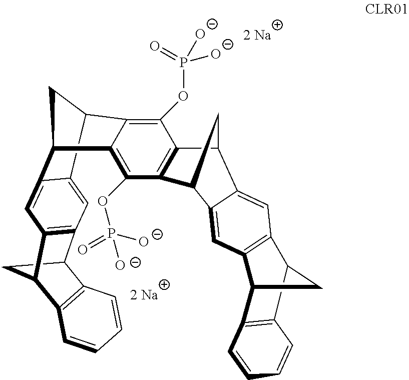

The above compounds may be obtained by methods known to skilled practitioners. In this regard, it is noted that 25-hydroxycholersterol is commercially available, e.g., from Santa Cruz Biotechnology (Dallas, TX) and methods of synthesizing molecular tweezers, including CLR01, are provided in U.S. Pat. No. 8,791,092.

Additional small organic molecule inhibitors can be identified by screening various chemical libraries for activity in inhibiting CRYAB expression and/or aggregation. In various embodiments, such small molecule inhibitors of CRYAB can be isolated from natural sources (for example, plants, fungi, microbes and the like) or isolated from random or combinatorial chemical libraries of synthetic or natural compounds, or synthesized (see, e.g., Werner et al., (2006) Brief Funct. Genomic Proteomic 5(1): 32-36). Many random or combinatorial libraries are known in the art that can be used. Numerous means are currently used for random and directed synthesis of libraries of small organic molecules. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from, e.g., Pan Laboratories (Bothell, Wash.) or MycoSearch (N.C.), or are readily producible. Additionally, natural and synthetically produced libraries and compounds are readily modified through conventional chemical, physical, and biochemical means (see, e.g., Blondelle et al., (1996) Tib Tech 14: 60).

Screening the libraries can be accomplished by any variety of commonly known methods (see, e.g., Inglese et al. (2006) Proc. Natl. Acad. Sci. USA, 103(31): 11473-11478; Inglese & Auld (2009) Application of High Throughput Screening (HTS) Techniques: Applications in Chemical Biology in Wiley Encyclopedia of Chemical Biology (Wiley & Sons, Inc., Hoboken, NJ); Macarron et al. (2011) Nat. Rev. Drug Discov. 10(3): 188-195; and the like). Additionally both virtual and real screening services are commercially available from a number of suppliers (see, e.g., ChemDiv (San Diego, CA), and the like).

Identification and screening of inhibitors of CRYAB expression and/or aggregation can be further facilitated by X-ray crystallography, neutron diffraction, nuclear magnetic resonance spectrometry, and other techniques for structure determination. These techniques provide for the rational design or identification of inhibitors. Methods for screening and identifying CRYAB inhibitors are known in the art, and the invention contemplates any inhibitor screening and identification approaches, now known or later developed, in the art.

### Inhibitory Nucleic Acids.

In certain embodiments, the inhibitor of CRYAB expression and/or activity comprises nucleic acid based inhibitors. Non-limiting examples of nucleic acid inhibitors of CRYAB expression include, but are not limited to, interfering RNA molecules (e.g., shRNA or siRNA), dsRNA, RNA polymerase III transcribed DNAs, antisense nucleic acids, ribozymes, and the like.

RNA interference (RNAi) is a process of sequence-specific post-transcriptional gene silencing by which double stranded RNA (dsRNA) homologous to a target locus can specifically inactivate gene function (see, e.g., Hammond et al. (2001) Nat. Genet. 2: 110-119; Sharp (1999) Genes Dev. 13: 139-141; and the like). This dsRNA-induced gene silencing is mediated by short double-stranded small interfering RNAs (siRNAs) generated from longer dsRNAs by ribonuclease III cleavage (see, e.g., Bernstein et al. (2001) Nature, 409: 363-366; Elbashir et al. (2001) Genes Dev. 15: 188-200; and the like). RNAi-mediated gene silencing is thought to occur via sequence-specific RNA degradation, wherein sequence specificity is determined by the interaction of an siRNA with its complementary sequence within a target RNA (see, e.g., Tuschl (2001) Chem. Biochem. 2: 239-245). RNAi can be activated by introduction of siRNAs (see, e.g., Elbashir et al. (2001) Nature, 411: 494-498) or short hairpin RNAs (shRNAs) bearing a fold back stem-loop structure (see, e.g., Paddison et al. (2002) Genes Dev. 16: 948-958; Sui et al. (2002) Proc. Natl. Acad. Sci. USA, 99: 5515-5520; Brummelkamp et al. (2002) Science, 296: 550-553; Paul et al. (2002) Nature Biotechnol. 20: 505-508; and the like).

shRNA/siRNA comprises a double stranded structure typically containing 15 to 50 base pairs and preferably 21 to 25 base pairs and having a nucleotide sequence identical or nearly identical to an expressed target gene (e.g., CRYAB) or RNA within the cell. In certain embodiments, the siRNA/shRNA inhibitors are preferably short double stranded nucleic acid duplexes (or stem-loop structures in case of shRNA) comprising annealed complementary single stranded nucleic acid molecules. However, embodiments in which the siRNAs comprise an annealed RNA:DNA duplex, wherein the sense strand of the duplex is a DNA molecule and the antisense strand of the duplex is a RNA molecule are also contemplated. In some embodiments, duplexed siRNAs have a 2 or 3 nucleotide 3′ overhang on each strand of the duplex. In some embodiments, siRNAs/shRNAs have 5′-phosphate and 3′-hydroxyl groups.

In various embodiments, the siRNAs may be introduced to a target cell as an annealed duplex siRNA, or as single stranded sense and antisense nucleic acid sequence that, once within the target cell, anneals to form the siRNA duplex. Alternatively, the sense and antisense strands of the siRNA may be encoded on an expression construct that is introduced to the target cell. Upon expression within the target cell, the transcribed sense and antisense strands may anneal to reconstitute the siRNA. 100% sequence complementarity between the siRNA and the target nucleic acid is not required to practice the methods described herein. Expression of shRNA in cells can be obtained by delivery of plasmids or through viral or bacterial vectors. A variety of viral vectors can be used to obtain shRNA expression in cells including adeno-associated viruses (AAVs), adenoviruses, retroviruses, and lentiviruses.

Antisense oligonucleotides, including antisense DNA, RNA, and DNA/RNA molecules, act to directly block the translation of mRNA by binding to targeted mRNA and preventing protein translation. Preferably, antisense oligonucleotides are of at least about 15 bases and are complementary to unique regions of the target DNA sequence. Such antisense oligonucleotides can be synthesized, e.g., by conventional techniques (see, e.g., Dallas et al., (2006) Med. Sci. Monit. 12(4): RA67-74; Kalota et al. (2006) Handb. Exp. Pharmacol. 173: 173-96; Lutzelburger et al. (2006) Handb. Exp. Pharmacol. 173: 243-259; and the like).

RNA polymerase III-transcribed DNAs contain promoters, such as the U6 promoter. These DNAs can be transcribed to produce small hairpin RNAs in the cell that can function as siRNA or linear RNAs that can function as antisense RNA. The inhibitor may be polymerized in vitro, recombinant RNA, contain chimeric sequences, or derivatives of these groups. The inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded (see, e.g., Bass (2001) Nature, 411: 428 429; Elbashir et al. (2001) Nature, 411: 494 498; and PCT Publication Nos: WO 00/044895, WO 01/036646, WO 99/032619, WO 00/001846, WO 01/029058, WO 99/007409, WO 00/044914).

Aptamer nucleic acid sequences are readily made that bind to a wide variety of target molecules. The aptamer nucleic acid sequences useful in the methods described herein can be comprised entirely of RNA or partially of RNA, or entirely or partially of DNA and/or other nucleotide analogs. Aptamers are typically developed to bind particular ligands by employing known in vivo or in vitro (most typically, in vitro) selection techniques known as SELEX (Systematic Evolution of Ligands by Exponential Enrichment). Methods of making aptamers are described in, for example, Ellington & Szostak (1990) Nature, 346: 818, Tuerk & Gold (1990) Science, 249: 505, U.S. Pat. No. 5,582,981; PCT Publication No. WO 00/020040; U.S. Pat. No. 5,270,163; Lorsch & Szostak (1994) Biochem. 33: 973; Mannironi et al., (1997) Biochem. 36: 9726; Blind (1999) Proc. Natl. Acad. Sci. USA, 96:3606-3610; Huizenga & Szostak (1995) Biochem. 34: 656-665; PCT Publication Nos. WO 99/054506, WO 99/027133, and WO 97/042317; and U.S. Pat. No. 5,756,291.

In various embodiments, nucleic acid-based inhibitors used in the methods described herein may include one or more modifications, e.g., to increase intracellular stability and efficacy (e.g., modifications to the base moiety, sugar moiety, phosphate moiety, phosphate-sugar backbone, or a combination thereof). For example, the phosphodiester linkages may be modified to include at least one heteroatom other than oxygen, such as nitrogen or sulfur. In this case, for example, the phosphodiester linkage may be replaced by a phosphothioester linkage. Similarly, bases may be modified to block the activity of adenosine deaminase. Other examples of useful modifications are morpholino modifications and locked nucleic acids (LNA). The nucleic acid-based inhibitor molecule is produced synthetically, or by in vitro transcription. A modified nucleoside may be introduced during synthesis or transcription.

Non-limiting examples of modified base moieties include inosine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methyl cytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyl adenine, uracil-5-oxyacetic acid (v), pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2, 6-diaminopurine.

Non-limiting examples of modified sugar moieties include arabinose, 2-fluoroarabinose, xylulose, and hexose. Modified siRNAs may contain substituted sugar moieties comprising one of the following at the 2′ position: OH, SH, SCH—, F, OCN, O(CH₂)ₙNH₂ or O(CH₂)·CH₃ where n is from 1 to about 10; C- to C₁₀ lower alkyl, substituted lower alkyl, alkaryl or aralkyl; Cl; Br; CN; CF₃; OCF₃; O—; S—, or N-alkyl; O—, S—, or N-alkenyl; SOCH₃; SO₂CH₃; ONO₂; NO₂; N₃; NH₂; heterocycloalkyl; heterocycloalkaryl; aminoalkylamino; polyalkylamino; substituted sialyl; a fluorescein moiety; a reporter group; a group for improving the pharmacokinetic properties; or a group for improving the pharmacodynamic properties, and other substituents having similar properties. Modified nucleic acid-based inhibitors may also have sugar mimetics such as cyclobutyls or other carbocyclics in place of the pentofuranosyl group.

Non-limiting examples of modifications of phosphate backbone include a phosphorothioate, a phosphorodithioate, a phosphoramidothioate, a phosphoramidate, a phosphordiamidate, a methylphosphonate, a phosphotriester, an alkyl phosphotriester, and a formacetal or analog thereof, as well as chimeras between methylphosphonate and phosphodiester, short chain alkyl, or cycloalkyl intersugar linkages or short chain heteroatomic or heterocyclic intersugar linkages. Specific non-limiting examples include those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—PO₂—O—CH₂). U.S. Pat. No. 5,677,437 describes heteroaromatic oligonucleoside linkages. Nitrogen linkers or groups containing nitrogen can also be used to prepare oligonucleotide mimics (U.S. Pat. Nos. 5,792,844 and 5,783,682). U.S. Pat. No. 5,637,684 describes phosphoramidate and phosphorothioamidate oligomeric compounds.

Also envisioned are modified nucleic acid-based inhibitors having morpholino backbone structures in which the bases are linked to 6-membered morpholine rings, that are connected to other morpholine-linked bases via non-ionic phosphorodiamidate intersubunit linkages. Morpholino derivatives are highly resistant to nucleases and have good targeting predictability (U.S. Pat. No. 5,034,506; Summerton (1999) Biochim. Biophys. Acta, 1489: 141-158; Summerton & Weller (1997) Antisense Nucleic Acid Drug Dev. 7: 187-195; Arora et al. (2000) J. Pharmacol. Exp. Ther. 292: 921-928; Qin et al. (2000) Antisense Nucleic Acid Drug Dev. 10: 11-16; Heasman et al. (2000) Dev. Biol. 222: 124-134; Nasevicius & Ekker (2000) Nat. Genet. 26: 216-220).

Another type of a useful modification is the peptide-nucleic acid (PNA) backbone: the phosphodiester backbone of the oligonucleotide may be replaced with a polyamide backbone, the bases being bound directly or indirectly to the aza nitrogen atoms of the polyamide backbone (see, e.g., Nielsen et al. (1991) Science, 254: 1497).

In certain embodiments, locked nucleic acids (LNA) can be used (reviewed in, e.g., Jepsen & Wengel (2004) Curr. Opin. Drug Discov. Devel. 7: 188-194; Crinelli et al. (2004) Curr. Drug Targets, 5: 745-752). LNA are nucleic acid analog(s) with a 2′-O, 4′-C methylene bridge. This bridge restricts the flexibility of the ribofuranose ring and locks the structure into a rigid C3-endo conformation, conferring enhanced hybridization performance and exceptional biostability.

Modified nucleic acid-based inhibitors can include appending groups such as, e.g., peptides, or agents facilitating transport across the cell membrane (see, e.g., Letsinger et al. (1989) Proc. Natl. Acad. Sci. USA, 86: 6553-6556; Lemaitre et al. (1987) Proc. Natl. Acad. Sci. USA, 84: 648-652; PCT Publication No. WO 88/009810), or blood-brain barrier (see, e.g., PCT Publication No. WO 89/010134), etc.

In various embodiments, nucleic acid-based inhibitors used in the methods described herein can be synthesized by standard methods known in the art, e.g., by use of an automated synthesizer. In one illustrative, but non-limiting embodiment, RNA molecules can be chemically synthesized using appropriately protected ribonucleoside phosphoramidites and a conventional DNA/RNA synthesizer. In case of siRNA molecules, following chemical synthesis, single stranded RNA molecules are deprotected, annealed to form siRNAs, and purified (e.g., by gel electrophoresis or HPLC). Commercial suppliers of synthetic RNA molecules or synthesis reagents include, e.g., Proligo (Hamburg, Germany), Dharmacon Research (Lafayette, Colo.), Pierce Chemical (part of Perbio Science, Rockford, Ill.), Glen Research (Sterling, Va.), ChemGenes (Ashland, Mass.) and Cruachem (Glasgow, UK).

Alternatively, standard procedures may be used for in vitro transcription of RNA from DNA templates carrying RNA polymerase promoter sequences (e.g., T7 or SP6 RNA polymerase promoter sequences) (see, e.g., Donze & Picard (2002) Nucleic Acids Res. 30: e46; Yu et al. (2002) Proc. Natl. Acad. Sci. USA, 99: 6047-6052; Weintraub et al. (1986) Trends in Genetics, 1:1). In the case of siRNA molecules, the sense and antisense transcripts may be synthesized in two independent reactions and annealed later, or may be synthesized simultaneously in a single reaction. siRNA molecules may be formed within a cell by transcription of RNA from an expression construct introduced into the cell. For example, both a protocol and an expression construct for in vivo expression of siRNAs are described in Yu et al., supra.

In various embodiments, the expression constructs for in vivo production of nucleic acid-based inhibitors of the invention comprise encoding sequences operably linked to elements necessary for the proper transcription, including promoter elements and transcription termination signals. Non-limiting examples of promoters for use in such expression constructs include the polymerase-III H1-RNA promoter (see, e.g., Brummelkamp et al., supra) and the U6 polymerase-III promoter (see, e.g., Sui et al., supra; Paul, et al. supra; and Yu et al., supra). The expression constructs can further comprise vector sequences that facilitate the cloning of the expression constructs. Standard vectors that may be used in practicing the methods described herein are known in the art.

Additionally a number of shRNA constructs that knock down human CRYAB are known to those of skill in the art and are commercially available. Such constructs include, but are not limited to GIPZ Lentiviral Human CRYAB shRNA, SMARTvector Inducible Human CRYAB shRNA, TRC Lentiviral Human CRYAB shRNA, and TRIPZ Inducible Lentiviral Human CRYAB shRNA all available from Dharmacon, Inc. (Lafayette, CO).

### Gene Editing Nucleases (Targeted Endonucleases).

In certain embodiments, inhibition of CRYAB expression can be accomplished by the use of genome editing methods such as, e.g., methods that involve the use of Clustered Regularly Interspaced Short Palindromic Repeats (CRISPR)/Cas9 gene systems, methods that involve the use of zinc finger nucleases (ZFNs), methods that involve the use of transcription activator-like effector nucleases (TALENs), as well as methods that involve the use of any other nucleases that can cause DNA breaks or bind to DNA. Genome editing tools such as TALENs, ZFNs, and CRISPR/Cas9 system are examples of targeted nuclease systems: these systems have a DNA-binding member that localizes the nuclease to a target site. The site is then cut by the nuclease. TALENs and ZFNs have the nuclease fused to the DNA-binding member. CRISPR/Cas9 are cognates that find each other on the target DNA. The DNA-binding member is typically designed in light of the intended cognate sequence so as to obtain a nucleolytic action at or near an intended site. Methods of utilizing these gene editing tools for inhibiting (e.g., knocking down or knocking out) gene expression (e.g., expression of the CRYAB gene) can be found, inter alia, in WO 2013163628, US 20140273235, EP 2336362, WO 2014093479, WO 2014089290, U.S. Pat. No. 8,795,965, US 20140357530, WO 2011091324, U.S. Pat. No. 8,106,255, US 20120192298, US 20110023159, US20110281306, and the like.

In various embodiments, targeted endonucleases (e.g., TALENs, CRISPR/Cas, Zinc Finger endonucleases, etc.) are used to cut out or to insert an inactivating donor cDNA into the gene (e.g., CRYAB) in a targeted manner. In various embodiments, the targeted endonucleases can be introduced into the cell as a protein or as a nucleic acid (e.g., an mRNA, or a vector) encoding the protein. In the case of CRISPR/Cas, the endonuclease can be introduced as a single nucleic acid encoding the CRISPR/Cas endonuclease and a guide RNA, as separate nucleic acids encoding the CRISPR/Cas endonuclease and the guide protein, or as a ribonucleoprotein (RNP) complex comprising the CRISPR/Cas endonuclease and the guide RNA (gRNA). In certain embodiments, the ribonucleoprotein (RNP) complex comprises the CRISPR/Cas endonuclease conjugated to the guide RNA (gRNA).

Methods of introducing targeted endonucleases (and guide RNAs when required) into the cells (transecting the cells) are well known to those of skill in the art. Such methods include, but are not limited to electroporation, sonoporation, cell squeezing, optical transfection, impalefection, and the like. Electroporation is a particularly suitable method (see, e.g., Example 1) where transient increase in the permeability of cell membrane is achieved when the cells are exposed to short pulses of an intense electric field. Cell squeezing enables delivery of molecules into cells via cell membrane deformation. It is a high throughput vector-free microfluidic platform for intracellular delivery. It reduces the possibility of toxicity or off-target effects as it does not rely on exogenous materials or electrical fields (e.g., Sharei et al. (2013) Proc. Natl. Acad. Sci. USA, 110 (6): 2082-2087). Sonoporation uses high-intensity ultrasound to induce pore formation in cell membranes. Optical transfection is a method where a tiny (˜1 μm diameter) hole is transiently generated in the plasma membrane of a cell using a highly focused laser (see, e.g., Tsukakoshi et al. (1984) Appl. Physics B: Photophysics and Laser Chem. 35 (3): 135-140). Impalefection is a method of introducing a nucleic acid bound to a surface of a nanofiber that is inserted into a cell. This approach can also be implemented with arrays of nanofibers that are introduced into large numbers of cells and intact tissue.

Illustrative, but non-limiting, chemical-based transfection methods include, but are not limited to, calcium phosphate, transfection, dendrimers-based transfection, the use of cationic polymers (e.g., DEAE-dextran or polyethylenimine (PEI)), lipofection (e.g., using a positively charged (cationic) lipid or cationic liposomes, or mixtures thereof), or a non-liposomal transfection reagent such as FUGENE®.

Illustrative, but non-limiting, particle-based transfection methods include, but are not limited to the gene gun, where the DNA is coupled to a nanoparticle of an inert solid (commonly gold), which is then “shot” directly into the target cell's nucleus, magnetofection, or magnet-assisted transfection, where magnetic force is used to deliver nucleic acids into target cells, and the like.

These methods of introducing the endonuclease, and/or nucleic acid encoding an endonuclease, and/or a gRNA (where required) are illustrative and non-limiting. Using the teachings provided herein, numerous other approaches will be available to one of skill in the art.

### TALENs

In certain embodiments, the targeting endonuclease can be a Transcription Activator-Like Effector Nuclease (TALEN). TAL effector nucleases are a class of sequence-specific nucleases derived from Xanthomonas bacteria, that can be used to make double-strand breaks at specific target sequences in the genome of a prokaryotic or eukaryotic organism. The DNA binding domain of the TAL effector contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence.

TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences (see, e.g., WO 2010/079430; Morbitzer et al. (2010) Proc. Natl. Acad. Sci. USA, 107(50): 21617-21622; Scholze & Boch (2010) Virulence, 1: 428-432; Christian et al. (2010) Genetics, 186:757-761; Li et al. (2010) Nucl. Acids Res. (1):359-372; and Miller et al. (2011) Nat. Biotechl. 29: 143-148).

To produce a TALEN, a TAL protein is fused to a nuclease, which is typically a wild-type or mutated FokI endonuclease. Several mutations to FokI have been made for its use in TALENs. These, for example, improve cleavage specificity or activity (see, e.g., Cermak et al. (2011) Nucl. Acids Res. 39: e82; Miller et al. (2011) Nat. Biotech. 29: 143-148; Hockemeyer et al. (2011) Nat. Biotech. 29: 731-734; Wood et al. (2011) Science, 333: 307; Doyon et al. (2010) Nat. Meth. 8: 74-79; Szczepek et al. (2007) Nat. Biotech. 25: 786-793; and Guo et al. (2010) J. Mol. Biol. 200: 96).

The FokI domain functions as a dimer, typically requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity (see, e.g., Miller et al. (2011) Nat. Biotech., 29: 143-148).

Examples of suitable TAL nucleases, and methods for preparing suitable TAL nucleases, are disclosed, e.g., in US Patent Application Nos. 2011/0239315 A1, 2011/0269234 A1, 2011/0145940 A1, 2003/0232410 A1, 2005/0208489 A1, 2005/0026157 A1, 2005/0064474 A1, 2006/0188987 A1, and 2006/0063231 A1. In various embodiments, TAL effector nucleases are engineered that cut in or near a target nucleic acid sequence in, e.g., a genomic locus of interest (e.g., in the CRYAB gene), where the target nucleic acid sequence is at or near a sequence to be modified by a targeting vector (e.g., CRYAB gene). The TAL nucleases suitable for use with the various methods and compositions provided herein include those that are specifically designed to bind at or near target nucleic acid sequences to be modified by targeting vectors as described herein.

In one illustrative, but non-limiting embodiment, each monomer of the TALEN comprises 10 or more DNA binding repeats, and in some cases 15 or more DNA binding repeats (e.g., in certain embodiments, 12-25 TAL repeats), wherein each TAL repeat binds a 1 bp subsite. In one embodiment, the nuclease agent is a chimeric protein comprising a TAL repeat-based DNA binding domain operably linked to an independent nuclease. In one embodiment, the independent nuclease is a Fok1 endonuclease (see e.g., Kim et al. (1996) Proc. Natl. Acad. Sci. USA, 93:1156-1160), however, other useful endonucleases may include, but are not limited to, for example, HhaI, HindIII, Nod, BbvCI, EcoRI, BglI, and AlwI.

In some embodiments, the TAL effector domain that binds to a specific nucleotide sequence within the target DNA (e.g., CRYAB gene) comprises a plurality of repeat variable-diresidues (RVD) each of which determines recognition of a base pair in the target DNA sequence, where each DNA binding repeat is responsible for recognizing one base pair in the target DNA sequence, and wherein the RVD comprises one or more of: HD for recognizing C; NG for recognizing T; NI for recognizing A; NN for recognizing G or A; NS for recognizing A or C or G or T; N* for recognizing C or T, where * represents a gap in the second position of the RVD; HG for recognizing T; H* for recognizing T, where * represents a gap in the second position of the RVD; IG for recognizing T; NK for recognizing G; HA for recognizing C; ND for recognizing C; HI for recognizing C; HN for recognizing G; NA for recognizing G; SN for recognizing G or A; and YG for recognizing T.

If the genome editing endonuclease to be utilized is a TALEN, in some embodiments, optimal target sites may be selected in accordance with the methods described by Sanjana et al. (2012) Nat. Protocol., 7: 171-192. In brief, in various embodiments, TALENs function as dimers, and a pair of TALENs, referred to as the left and right TALENs, target sequences on opposite strands of DNA. TALENs can be engineered as a fusion of the TALE DNA-binding domain and a monomeric FokI catalytic domain. In certain embodiments, to facilitate FokI dimerization, the left and right TALEN target sites can be chosen with a spacing of approximately 14-20 bases. Therefore, for a pair of TALENs, each targeting 20-bp sequences, an optimal target site can have the form 5′-TN¹⁹N¹⁴⁻²⁰N¹⁹A-3′, where the left TALEN targets 5′-TN¹⁹-3′ and the right TALEN targets the antisense strand of 5′-N¹⁹A-3′ (N=A, G, T or C). This is of course illustrative and non-limiting and TALENs that bind to the CRYAB gene are readily created. For more information on TALENs, refer to U.S. Pat. No. 8,685,737.

### CRISPR/Cas Systems.

In certain embodiments, the targeting endonuclease can comprise a CRISPR/Cas endonuclease which is typically guided to a target site by one or more guide RNAs (gRNAs). CRISPR-based endonucleases are RNA-guided endonucleases derived from CRISPR/Cas systems. Bacteria and archaea have evolved an RNA-based adaptive immune system that uses CRISPR (clustered regularly interspersed short palindromic repeat) and Cas (CRISPR-associated) proteins to detect and destroy invading viruses or plasmids. CRISPR/Cas endonucleases can be programmed to introduce targeted site-specific double-strand breaks by providing target-specific synthetic guide RNAs (see, e.g., Jinek et al. (2012) Science, 337: 816-821).

In various embodiments, the CRISPR-based endonuclease can be derived from a CRISPR/Cas type I, type II, type III, type V, or type VI system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

### Type II CRISPR/Cas Endonucleases (e.g., Cas 9)

In certain embodiments, the CRISPR-based endonuclease is derived from a type II CRISPR/Cas system. In illustrative, but non-limiting embodiments, the CRISPR-based endonuclease is derived from a Cas9 protein. In certain embodiments, the Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. In one specific illustrative embodiment, the CRISPR-based nuclease is derived from a Cas9 protein from Streptococcus pyogenes.

In general, CRISPR/Cas proteins comprise at least one RNA recognition and/or RNA binding domain. RNA recognition and/or RNA binding domains interact with the guide RNA such that the CRISPR/Cas protein is directed to a specific genomic or genomic sequence. CRISPR/Cas proteins can also comprise nuclease domains (i.e., DNase or RNase domains), DNA binding domains, helicase domains, protein-protein interaction domains, dimerization domains, as well as other domains.

The CRISPR-based endonuclease used herein can be a wild type CRISPR/Cas protein, a modified CRISPR/Cas protein, or a fragment of a wild type or modified CRISPR/Cas protein. In certain embodiments, the CRISPR/Cas protein can be modified to increase nucleic acid binding affinity and/or specificity, alter an enzymatic activity, and/or change another property of the protein. For example, the CRISPR/Cas protein can be truncated to remove domains that are not essential for the function of the protein. The CRISPR/Cas protein also can be truncated or modified to optimize the activity of the protein or an effector domain fused with the CRISPR/Cas protein.

In some embodiments, the CRISPR-based endonuclease can be derived from a wild type Cas9 protein or fragment thereof. In other embodiments, the CRISPR-based endonuclease can be derived from a modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-strand break in DNA (see, e.g., Jinek et al. (2012) Science, 337: 816-821). In one embodiment, the CRISPR-based endonuclease is derived from a Cas9 protein and comprises two function nuclease domains, which together introduce a double-stranded break into the targeted site.

The target sites recognized by naturally occurring CRISPR/Cas systems typically having lengths of about 14-15 bp (see, e.g., Cong et al. (2013) Science, 339: 819-823). The target site has no sequence limitation except that sequence complementary to the 5′ end of the guide RNA (i.e., called a protospacer sequence) is typically immediately followed by (3′ or downstream) a consensus sequence. This consensus sequence is also known as a protospacer adjacent motif (or PAM). Examples of PAM include, but are not limited to, NGG, NGGNG, and NNAGAAW (wherein N is defined as any nucleotide and W is defined as either A or T). At the typical length, only about 5-7% of the target sites would be unique within a target genome, indicating that off target effects could be significant. The length of the target site can be expanded by requiring two binding events. For example, CRISPR-based endonucleases can be modified such that they can only cleave one strand of a double-stranded sequence (i.e., converted to nickases). Thus, the use of a CRISPR-based nickase in combination with two different guide RNAs would essentially double the length of the target site, while still effecting a double stranded break.

The requirement of the crRNA-tracrRNA complex in a CRISPR/Cas system can be avoided by use of an engineered “single-guide RNA” (sgRNA) that comprises the hairpin normally formed by the annealing of the crRNA and the tracrRNA (see Jinek et al. (2012) Science 337:816; Cong et al. (2013) Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA containing a PAM sequence has been used for RNA guided genome editing and has been useful for zebrafish embryo genomic editing in vivo (see Hwang et al. (2013) Nat. Biotechnol., 31(3):227) with editing efficiencies similar to ZFNs and TALENs.

Accordingly in certain embodiments, a CRISPR/Cas endonuclease complex used in the methods described herein comprises a Cas protein and at least one to two ribonucleic acids (e.g., gRNAs) that are capable of directing the Cas protein to and hybridizing to a target motif of a target polynucleotide sequence (e.g., CRYAB). In some embodiments, a CRISPR/Cas endonuclease complex used in the methods described herein comprises a Cas protein and one ribonucleic acid (e.g., gRNA) that us capable of directing the Cas protein to and hybridizing to a target motif of a target polynucleotide sequence.

As used herein, “protein” and “polypeptide” are used interchangeably to refer to a series of amino acid residues joined by peptide bonds (i.e., a polymer of amino acids) and include modified amino acids (e.g., phosphorylated, glycated, glycosolated, etc.) and amino acid analogs. Illustrative polypeptides or proteins include gene products, naturally occurring proteins, homologs, paralogs, fragments and other equivalents, variants, and analogs of the above.

In some embodiments, a Cas protein comprises a core Cas protein. Illustrative Cas core proteins include, but are not limited to, Cas1, Cas2, Cas3, Cas4, Cas5, Cas6, Cas7, Cas8 and Cas9. In some embodiments, a Cas protein comprises a Cas protein of an E. coli subtype (also known as CASS2). Illustrative Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Illustrative Cas proteins of the Ypest subtype include, but are not limited to Csy1, Csy2, Csy3, and Csy4. In some embodiments, a Cas protein comprises a Cas protein of the Nmeni subtype (also known as CASS4). Illustrative Cas proteins of the Nmeni subtype include, but are not limited to Csn1 and Csn2. In some embodiments, a Cas protein comprises a Cas protein of the Dvulg subtype (also known as CASS1). Illustrative Cas proteins of the Dvulg subtype include Csd1, Csd2, and Cas5d. In some embodiments, a Cas protein comprises a Cas protein of the Tneap subtype (also known as CASS7). Illustrative Cas proteins of the Tneap subtype include, but are not limited to, Cst1, Cst2, Cas5t. In some embodiments, a Cas protein comprises a Cas protein of the Hmari subtype. Illustrative Cas proteins of the Hmari subtype include, but are not limited to Csh1, Csh2, and Cas5h. In some embodiments, a Cas protein comprises a Cas protein of the Apern subtype (also known as CASS5). Illustrative Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Illustrative Cas proteins of the Mtube subtype include, but are not limited to Csm1, Csm2, Csm3, Csm4, and Csm5. In some embodiments, a Cas protein comprises a RAMP module Cas protein. Illustrative RAMP module Cas proteins include, but are not limited to, Cmr1, Cmr2, Cmr3, Cmr4, Cmr5, and Cmr6.

In some embodiments, the Cas protein is a Streptococcus pyogenes Cas9 protein (spCas9) or a functional portion thereof (see, e.g., UniProtKB—Q99ZW2 (CAS9_STRP1)). In some embodiments, the Cas protein is a Staphylococcus aureus Cas9 protein (saCas9) or a functional portion thereof. In some embodiments, the Cas protein is a Streptococcus thermophilus Cas9 protein (stCas9) or a functional portion thereof. In some embodiments, the Cas protein is a Neisseria meningitides Cas9 protein (nmCas9) or a functional portion thereof. In some embodiments, the Cas protein is a Treponema denticola Cas9 protein (tdCas9) or a functional portion thereof. In some embodiments, the Cas protein is Cas9 protein from any other bacterial species or functional portion thereof.

### Type V and Type VI CRISPR/Cas Endonucleases

In certain embodiments, the CRISPR/Cas endonuclease systems contemplated herein include, but are not limited to a type V or type VI CRISPR/Cas endonuclease (e.g., the genome editing endonuclease is a type V or type VI CRISPR/Cas endonuclease) (e.g., Cpf1, C2c1, C2c2, C2c3). Type V and type VI CRISPR/Cas endonucleases are a type of class 2 CRISPR/Cas endonuclease. Examples of type V CRISPR/Cas endonucleases include but are not limited to: Cpf1, C2c1, and C2c3. An example of a type VI CRISPR/Cas endonuclease is C2c2. In some cases, a subject genome targeting composition includes a type V CRISPR/Cas endonuclease (e.g., Cpf1, C2c1, C2c3). In some cases, a Type V CRISPR/Cas endonuclease is a Cpf1 protein. In some cases, a subject genome targeting composition includes a type VI CRISPR/Cas endonuclease (e.g., C2c2)

Like type II CRISPR/Cas endonucleases, type V and VI CRISPR/Cas endonucleases form a complex with a corresponding guide RNA. The guide RNA provides target specificity to an endonuclease-guide RNA RNP complex by having a nucleotide sequence (a guide sequence) that is complementary to a sequence (the target site) of a target nucleic acid (as described elsewhere herein). The endonuclease of the complex provides the site-specific activity. In other words, the endonuclease is guided to a target site (e.g., stabilized at a target site) within a target nucleic acid sequence (e.g., a chromosomal sequence) by virtue of its association with the protein-binding segment of the guide RNA.

Examples and guidance related to type V and type VI CRISPR/Cas proteins (e.g., cpf1, C2c1, C2c2, and C2c3 guide RNAs) can be found in the art (see, e.g., Zetsche et al. (2015) Cell, 163(3):759-771; Makarova et al. (2015) Nat. Rev. Microbiol. 13(11): 722-736; Shmakov et al. (2015) Mol. Cell, 60(3):385-397; and the like).

In some cases, the Type V or type VI CRISPR/Cas endonuclease (e.g., Cpf1, C2c1, C2c2, C2c3) is enzymatically active, e.g., the Type V or type VI CRISPR/Cas polypeptide, when bound to a guide RNA, and cleaves a target nucleic acid. In some cases, the Type V or type VI CRISPR/Cas endonuclease (e.g., Cpf1, C2c1, C2c2, C2c3) exhibits reduced enzymatic activity relative to a corresponding wild-type a Type V or type VI CRISPR/Cas endonuclease (e.g., Cpf1, C2c1, C2c2, C2c3), and retains DNA binding activity.

In some cases a type V CRISPR/Cas endonuclease is a Cpf1 protein or a functional portion thereof (see, e.g., UniProtKB—AOQ7Q2 (CPF1_FRATN)). Cpf1 protein is a member of the type V CRISPR system and is a polypeptide comprising about 1300 amino acids. Cpf1 contains a RuvC-like endonuclease domain. Unlike Cas9, Cpf1 cleaves target DNA in a staggered pattern using a single ribonuclease domain. The staggered DNA double-stranded break results in a 4 or 5-nt 5′ overhang.

The CRISPR-Cpf1 system, identified in Francisella spp, is a class 2 CRISPR-Cas system that mediates robust DNA interference in human cells. Although functionally conserved, Cpf1 and Cas9 differ in many aspects including in their guide RNAs and substrate specificity (see, e.g., Fagerlund et al. (2015) Genom. Bio. 16: 251). A major difference between Cas9 and Cpf1 proteins is that Cpf1 does not utilize tracrRNA, and thus requires only a crRNA. The FnCpf1 crRNAs are 42-44 nucleotides long (19-nucleotide repeat and 23-25-nucleotide spacer) and contain a single stem-loop, which tolerates sequence changes that retain secondary structure. In addition, the Cpf1 crRNAs are significantly shorter than the ˜100-nucleotide engineered sgRNAs required by Cas9, and the PAM requirements for FnCpf1 are 5′-TTN-3′ and 5′-CTA-3′ on the displaced strand. Although both Cas9 and Cpf1 make double strand breaks in the target DNA, Cas9 uses its RuvC- and HNH-like domains to make blunt-ended cuts within the seed sequence of the guide RNA, whereas Cpf1 uses a RuvC-like domain to produce staggered cuts outside of the seed. Because Cpf1 makes staggered cuts away from the critical seed region, NHEJ will not disrupt the target site, therefore ensuring that Cpf1 can continue to cut the same site until the desired HDR recombination event has taken place. Thus, in the methods and compositions described herein, it is understood that the term “Cas” includes both Cas9 and Cfp1 proteins. Accordingly, as used herein, a “CRISPR/Cas system” refers both CRISPR/Cas and/or CRISPR/Cfp1 systems, including both nuclease and/or transcription factor systems.

Accordingly, in certain embodiments, the methods described herein the Cas protein is Cpf1 from any bacterial species or functional portion thereof. In some aspects, Cpf1 is a Francisella novicida U112 protein or a functional portion thereof. In some aspects, Cpf1 is a Acidaminococcus sp. BV3L6 protein or a functional portion thereof. In some aspects, Cpf1 is a Lachnospiraceae bacterium ND2006 protein or a function portion thereof.

In certain embodiments, Cas protein may be a “functional portion” or “functional derivative” of a naturally occurring Cas protein, or of a modified Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity (e.g., endonuclease activity) in common with a corresponding native sequence polypeptide. As used herein, “functional portion” refers to a portion of a Cas polypeptide that retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cpf1 protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cpf1 protein comprises a functional portion of a RuvC-like domain.

In certain embodiments, a biological activity contemplated herein is the ability of the functional derivative to introduce a double strand break (DSB) at a desired target site in a genomic DNA (e.g., in the CRYAB gene). The term “derivative” encompasses both amino acid sequence variants of polypeptide, covalent modifications, and fusions thereof. In some aspects, a functional derivative may comprise a single biological property of a naturally occurring Cas protein. In other aspects, a function derivative may comprise a subset of biological properties of a naturally occurring Cas protein.

In view of the foregoing, the term “Cas polypeptide” as used herein encompasses a full-length Cas polypeptide, an enzymatically active fragment of a Cas polypeptide, and enzymatically active derivatives of a Cas polypeptide or fragment thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically, recombinantly expressed, or by a combination of these procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In certain embodiments, the Cas proteins used in the constructs described herein may be mutated to alter functionality. Illustrative selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in WO 02/077227.

In certain embodiments, the Cas proteins (e.g., Cas9 protein) comprise truncated Cas proteins. In one illustrative, but non-limiting, embodiment, the Cas9 comprises only the domain responsible for interaction with the crRNA or sgRNA and the target DNA.

In certain embodiments, the Cas proteins comprising the constructs described herein comprise a Cas protein, or truncation thereof, fused to a different functional domain. In some aspects, the functional domain is an activation or a repression domain. In other aspects, the functional domain is a nuclease domain. In some embodiments, the nuclease domain is a FokI endonuclease domain (see, e.g. Tsai (2014) Nat. Biotechnol. doi:10.1038/nbt.2908). In some embodiments, the FokI domain comprises mutations in the dimerization domain.

### Guide RNA (for CRISPR/Cas Endonucleases)

In various embodiments, the constructs methods described herein involve the introduction into the desired cell(s) of one or more guide RNAs (gRNAs) along with the CRISPR/Cas endonuclease. In certain embodiments, the CRISPR/Cas endonuclease and gRNA are encoded by a single nucleic acid that is introduced into the cell. In certain embodiments, the CRISPR/Cas endonuclease and gRNA are introduced into the cell as a ribonucleoprotein complex. In certain embodiments, the complex comprises a Cas protein attached to a single guide RNA.

A nucleic acid molecule that binds to a class 2 CRISPR/Cas endonuclease (e.g., a Cas9 protein; a type V or type VI CRISPR/Cas protein; a Cpf1 protein; etc.) and targets the complex to a specific location within a target nucleic acid is referred to herein as a “guide RNA” or “CRISPR/Cas guide nucleic acid” or “CRISPR/Cas guide RNA.”

In various embodiments, the guide RNA provides target specificity to the complex (the RNP complex) by including a targeting segment, which includes a guide sequence (also referred to herein as a targeting sequence), which typically comprises a nucleotide sequence that is complementary to a sequence of a target nucleic acid

A guide RNA can be referred to by the protein to which it corresponds. For example, when the class 2 CRISPR/Cas endonuclease is a Cas9 protein, the corresponding guide RNA can be referred to as a “Cas9 guide RNA.” Likewise, as another example, when the class 2 CRISPR/Cas endonuclease is a Cpf1 protein, the corresponding guide RNA can be referred to as a “Cpf1 guide RNA.”

In some embodiments, a guide RNA includes two separate nucleic acid molecules (or two sequenced within a single molecule): an “activator” and a “targeter” and is referred to herein as a “dual guide RNA”, a “double-molecule guide RNA”, a “two-molecule guide RNA”, or a “dgRNA.” In some embodiments, the guide RNA is one molecule (e.g., for some class 2 CRISPR/Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.”

### Cas9 Guide RNA

A nucleic acid molecule that binds to a Cas9 protein and targets the complex to a specific location (e.g., CRYAB gene) within a target nucleic acid is referred to herein as a “Cas9 guide RNA.” In certain embodiments, a Cas9 guide RNA (can be said to include two segments, a first segment (referred to herein as a “targeting segment”); and a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule.

In various embodiments, the first segment (targeting segment) of a Cas9 guide RNA typically includes a nucleotide sequence (a guide sequence) that is complementary to (and therefore hybridizes with) a specific sequence (a target site) within a target nucleic acid (e.g., a target ssRNA, a target ssDNA, the complementary strand of a double stranded target DNA, etc.). The protein-binding segment (or “protein-binding sequence”) interacts with (binds to) a Cas9 polypeptide. The protein-binding segment of a subject Cas9 guide RNA typically includes two complementary stretches of nucleotides that hybridize to one another to form a double stranded RNA duplex (dsRNA duplex). Site-specific binding and/or cleavage of a target nucleic acid (e.g., genomic DNA) can occur at locations (e.g., target sequence of a target locus) determined by base-pairing complementarity between the Cas9 guide RNA (the guide sequence of the Cas9 guide RNA) and the target nucleic acid

A Cas9 guide RNA and a Cas9 protein form a complex (e.g., bind via non-covalent interactions). The Cas9 guide RNA provides target specificity to the complex by including a targeting segment, which includes a guide sequence (a nucleotide sequence that is complementary to a sequence of a target nucleic acid). The Cas9 protein of the complex provides the site-specific activity (e.g., cleavage activity or an activity provided by the Cas9 protein when the Cas9 protein is a Cas9 fusion polypeptide, i.e., has a fusion partner). In other words, the Cas9 protein is guided to a target nucleic acid sequence (e.g., a target sequence in a chromosomal nucleic acid, e.g., a chromosome; a target sequence in an extrachromosomal nucleic acid, e.g., an episomal nucleic acid, a minicircle, an ssRNA, an ssDNA, etc.; a target sequence in a mitochondrial nucleic acid; a target sequence in a chloroplast nucleic acid; a target sequence in a plasmid; a target sequence in a viral nucleic acid; etc.) by virtue of its association with the Cas9 guide RNA.

The “guide sequence” also referred to as the “targeting sequence” of a Cas9 guide RNA can be modified so that the Cas9 guide RNA can target a Cas9 protein to any desired sequence of any desired target nucleic acid, with the exception that the protospacer adjacent motif (PAM) sequence can be taken into account. Thus, for example, a Cas9 guide RNA can have a targeting segment with a sequence (a guide sequence) that has complementarity with (e.g., can hybridize to) a sequence in a nucleic acid in a eukaryotic cell, e.g., a viral nucleic acid, a eukaryotic nucleic acid (e.g., a eukaryotic chromosome, chromosomal sequence, a eukaryotic RNA, etc.), and the like.

In some embodiments, a Cas9 guide RNA includes two separate nucleic acid molecules: an “activator” and a “targeter” and is referred to herein as a “dual Cas9 guide RNA”, a “double-molecule Cas9 guide RNA”, or a “two-molecule Cas9 guide RNA” a “dual guide RNA”, or a “dgRNA.” In some embodiments, the activator and targeter are covalently linked to one another (e.g., via intervening nucleotides) and the guide RNA is referred to as a “single guide RNA”, a “Cas9 single guide RNA”, a “single-molecule Cas9 guide RNA,” or a “one-molecule Cas9 guide RNA”, or simply “sgRNA.”

In various embodiments, a Cas9 guide RNA comprises a crRNA-like (“CRISPR RNA”/“targeter”/“crRNA”/“crRNA repeat”) molecule and a corresponding tracrRNA-like (“trans-acting CRISPR RNA”/“activator”/“tracrRNA”) molecule. A crRNA-like molecule (targeter) typically comprises both the targeting segment (single stranded) of the Cas9 guide RNA and a stretch (“duplex-forming segment”) of nucleotides that forms one half of the dsRNA duplex of the protein-binding segment of the Cas9 guide RNA. A corresponding tracrRNA-like molecule (activator/tracrRNA) typically comprises a stretch of nucleotides (duplex-forming segment) that forms the other half of the dsRNA duplex of the protein-binding segment of the guide nucleic acid. In other words, a stretch of nucleotides of a crRNA-like molecule are complementary to and hybridize with a stretch of nucleotides of a tracrRNA-like molecule to form the dsRNA duplex of the protein-binding domain of the Cas9 guide RNA. As such, each targeter molecule can be said to have a corresponding activator molecule (which has a region that hybridizes with the targeter). In various embodiments, the targeter molecule additionally provides the targeting segment. Thus, in various embodiments, a targeter and an activator molecule (as a corresponding pair) can hybridize to form a Cas9 guide RNA. The exact sequence of a given crRNA or tracrRNA molecule is characteristic of the species in which the RNA molecules are found. A subject dual Cas9 guide RNA can include any corresponding activator and targeter pair.

The term “activator” or “activator RNA” is used herein to mean a tracrRNA-like molecule (tracrRNA: “trans-acting CRISPR RNA”) of a Cas9 dual guide RNA (and therefore of a Cas9 single guide RNA when the “activator” and the “targeter” are linked together by, e.g., intervening nucleotides). Thus, for example, a Cas9 guide RNA (dgRNA or sgRNA) typically comprises an activator sequence (e.g., a tracrRNA sequence). A tracr molecule (a tracrRNA) is a naturally existing molecule that hybridizes with a CRISPR RNA molecule (a crRNA) to form a Cas9 dual guide RNA. The term “activator” is used herein to encompass naturally existing tracrRNAs, but also to encompass tracrRNAs with modifications (e.g., truncations, sequence variations, base modifications, backbone modifications, linkage modifications, etc.) where the activator retains at least one function of a tracrRNA (e.g., contributes to the dsRNA duplex to which Cas9 protein binds). In some cases the activator provides one or more stem loops that can interact with Cas9 protein. An activator can be referred to as having a tracr sequence (tracrRNA sequence) and in some cases is a tracrRNA, but the term “activator” is not limited to naturally existing tracrRNAs.

The term “targeter” or “targeter RNA” is used herein to refer to a crRNA-like molecule (crRNA: “CRISPR RNA”) of a Cas9 dual guide RNA (and therefore of a Cas9 single guide RNA when the “activator” and the “targeter” are linked together, e.g., by intervening nucleotides). Thus, for example, a Cas9 guide RNA (dgRNA or sgRNA) typically comprises a targeting segment (which includes nucleotides that hybridize with (are complementary to) a target nucleic acid, and a duplex-forming segment (e.g., a duplex forming segment of a crRNA, which can also be referred to as a crRNA repeat). Because the sequence of a targeting segment (the segment that hybridizes with a target sequence of a target nucleic acid) of a targeter is modified by a user to hybridize with a desired target nucleic acid, the sequence of a targeter will often be a non-naturally occurring sequence. However, in various embodiments, the duplex-forming segment of a targeter (described in more detail below), which hybridizes with the duplex-forming segment of an activator, can include a naturally existing sequence (e.g., can include the sequence of a duplex-forming segment of a naturally existing crRNA, which can also be referred to as a crRNA repeat). Thus, the term targeter is used herein to distinguish from naturally occurring crRNAs, despite the fact that part of a targeter (e.g., the duplex-forming segment) often includes a naturally occurring sequence from a crRNA. However, the term “targeter” encompasses naturally occurring crRNAs.

In various embodiments, a Cas9 guide RNA can also be said to include 3 parts: (i) a targeting sequence (a nucleotide sequence that hybridizes with a sequence of the target nucleic acid); (ii) an activator sequence (as described above)(in some cases, referred to as a tracr sequence); and (iii) a sequence that hybridizes to at least a portion of the activator sequence to form a double stranded duplex. A targeter has (i) and (iii); while an activator has (ii).

A Cas9 guide RNA (e.g., a dual guide RNA or a single guide RNA) can be comprised of any corresponding activator and targeter pair. In some cases, the duplex forming segments can be swapped between the activator and the targeter. In other words, in some cases, the targeter includes a sequence of nucleotides from a duplex forming segment of a tracrRNA (which sequence would normally be part of an activator) while the activator includes a sequence of nucleotides from a duplex forming segment of a crRNA (which sequence would normally be part of a targeter).

As noted above, a targeter typically comprises both the targeting segment (single stranded) of the Cas9 guide RNA and a stretch (“duplex-forming segment”) of nucleotides that forms one half of the dsRNA duplex of the protein-binding segment of the Cas9 guide RNA. A corresponding tracrRNA-like molecule (activator) typically comprises a stretch of nucleotides (a duplex-forming segment) that forms the other half of the dsRNA duplex of the protein-binding segment of the Cas9 guide RNA. In other words, a stretch of nucleotides of the targeter is complementary to and hybridizes with a stretch of nucleotides of the activator to form the dsRNA duplex of the protein-binding segment of a Cas9 guide RNA. As such, each targeter can be said to have a corresponding activator (which has a region that hybridizes with the targeter). The targeter molecule additionally provides the targeting segment. Thus, a targeter and an activator (as a corresponding pair) hybridize to form a Cas9 guide RNA. The particular sequence of a given naturally existing crRNA or tracrRNA molecule is characteristic of the species in which the RNA molecules are found. Examples of suitable activator and targeter are well known in the art.

In various embodiments, a Cas9 guide RNA (e.g., a dual guide RNA or a single guide RNA) can be comprised of any corresponding activator and targeter pair.

### Targeting Segment of a Cas9 Guide RNA

The first segment of a subject guide nucleic acid typically includes a guide sequence (e.g., a targeting sequence)(a nucleotide sequence that is complementary to a sequence (a target site) in a target nucleic acid). In other words, the targeting segment of a subject guide nucleic acid can interact with a target nucleic acid (e.g., double stranded DNA (dsDNA)) in a sequence-specific manner via hybridization (i.e., base pairing). As such, the nucleotide sequence of the targeting segment may vary (depending on the target) and can determine the location within the target nucleic acid that the Cas9 guide RNA and the target nucleic acid will interact. The targeting segment of a Cas9 guide RNA can be modified (e.g., by genetic engineering)/designed to hybridize to any desired sequence (target site) within a target nucleic acid (e.g., a eukaryotic target nucleic acid such as genomic DNA).

In certain embodiments, the targeting segment can have a length of 7 or more nucleotides (nt) (e.g., 8 or more, 9 or more, 10 or more, 12 or more, 15 or more, 20 or more, 25 or more, 30 or more, or 40 or more nucleotides). In some cases, the targeting segment can have a length of from 7 to 100 nucleotides (nt) (e.g., from 7 to 80 nt, from 7 to 60 nt, from 7 to 40 nt, from 7 to 30 nt, from 7 to 25 nt, from 7 to 22 nt, from 7 to 20 nt, from 7 to 18 nt, from 8 to 80 nt, from 8 to 60 nt, from 8 to 40 nt, from 8 to 30 nt, from 8 to 25 nt, from 8 to 22 nt, from 8 to 20 nt, from 8 to 18 nt, from 10 to 100 nt, from 10 to 80 nt, from 10 to 60 nt, from 10 to 40 nt, from 10 to 30 nt, from 10 to 25 nt, from 10 to 22 nt, from 10 to 20 nt, from 10 to 18 nt, from 12 to 100 nt, from 12 to 80 nt, from 12 to 60 nt, from 12 to 40 nt, from 12 to 30 nt, from 12 to 25 nt, from 12 to 22 nt, from 12 to 20 nt, from 12 to 18 nt, from 14 to 100 nt, from 14 to 80 nt, from 14 to 60 nt, from 14 to 40 nt, from 14 to 30 nt, from 14 to 25 nt, from 14 to 22 nt, from 14 to 20 nt, from 14 to 18 nt, from 16 to 100 nt, from 16 to 80 nt, from 16 to 60 nt, from 16 to 40 nt, from 16 to 30 nt, from 16 to 25 nt, from 16 to 22 nt, from 16 to 20 nt, from 16 to 18 nt, from 18 to 100 nt, from 18 to 80 nt, from 18 to 60 nt, from 18 to 40 nt, from 18 to 30 nt, from 18 to 25 nt, from 18 to 22 nt, or from 18 to 20 nt).

The nucleotide sequence (the targeting sequence) of the targeting segment that is complementary to a nucleotide sequence (target site) of the target nucleic acid can have a length of 10 nt or more. For example, the targeting sequence of the targeting segment that is complementary to a target site of the target nucleic acid can have a length of 12 nt or more, 15 nt or more, 18 nt or more, 19 nt or more, or 20 nt or more. In some cases, the nucleotide sequence (the targeting sequence) of the targeting segment that is complementary to a nucleotide sequence (target site) of the target nucleic acid has a length of 12 nt or more. In some cases, the nucleotide sequence (the targeting sequence) of the targeting segment that is complementary to a nucleotide sequence (target site) of the target nucleic acid has a length of 18 nt or more.

For example, in certain embodiments, the targeting sequence of the targeting segment that is complementary to a target sequence of the target nucleic acid can have a length of from 10 to 100 nucleotides (nt) (e.g., from 10 to 90 nt, from 10 to 75 nt, from 10 to 60 nt, from 10 to 50 nt, from 10 to 35 nt, from 10 to 30 nt, from 10 to 25 nt, from 10 to 22 nt, from 10 to 20 nt, from 12 to 100 nt, from 12 to 90 nt, from 12 to 75 nt, from 12 to 60 nt, from 12 to 50 nt, from 12 to 35 nt, from 12 to 30 nt, from 12 to 25 nt, from 12 to 22 nt, from 12 to 20 nt, from 15 to 100 nt, from 15 to 90 nt, from 15 to 75 nt, from 15 to 60 nt, from 15 to 50 nt, from 15 to 35 nt, from 15 to 30 nt, from 15 to 25 nt, from 15 to 22 nt, from 15 to 20 nt, from 17 to 100 nt, from 17 to 90 nt, from 17 to 75 nt, from 17 to 60 nt, from 17 to 50 nt, from 17 to 35 nt, from 17 to 30 nt, from 17 to 25 nt, from 17 to 22 nt, from 17 to 20 nt, from 18 to 100 nt, from 18 to 90 nt, from 18 to 75 nt, from 18 to 60 nt, from 18 to 50 nt, from 18 to 35 nt, from 18 to 30 nt, from 18 to 25 nt, from 18 to 22 nt, or from 18 to 20 nt). In some cases, the targeting sequence of the targeting segment that is complementary to a target sequence of the target nucleic acid has a length of from 15 nt to 30 nt. In some cases, the targeting sequence of the targeting segment that is complementary to a target sequence of the target nucleic acid has a length of from 15 nt to 25 nt. In some cases, the targeting sequence of the targeting segment that is complementary to a target sequence of the target nucleic acid has a length of from 18 nt to 30 nt. In some cases, the targeting sequence of the targeting segment that is complementary to a target sequence of the target nucleic acid has a length of from 18 nt to 25 nt. In some cases, the targeting sequence of the targeting segment that is complementary to a target sequence of the target nucleic acid has a length of from 18 nt to 22 nt. In some cases, the targeting sequence of the targeting segment that is complementary to a target site of the target nucleic acid is 20 nucleotides in length. In some cases, the targeting sequence of the targeting segment that is complementary to a target site of the target nucleic acid is 19 nucleotides in length.

In certain embodiments, the percent complementarity between the targeting sequence (guide sequence) of the targeting segment and the target site of the target nucleic acid can be 60% or more (e.g., 65% or more, 70% or more, 75% or more, 80% or more, 85% or more, 90% or more, 95% or more, 97% or more, 98% or more, 99% or more, or 100%). In some cases, the percent complementarity between the targeting sequence of the targeting segment and the target site of the target nucleic acid is 100% over the seven contiguous 5′-most nucleotides of the target site of the target nucleic acid. In some cases, the percent complementarity between the targeting sequence of the targeting segment and the target site of the target nucleic acid is 60% or more over about 20 contiguous nucleotides. In some cases, the percent complementarity between the targeting sequence of the targeting segment and the target site of the target nucleic acid is 100% over the fourteen contiguous 5′-most nucleotides of the target site of the target nucleic acid and as low as 0% or more over the remainder. In such a case, the targeting sequence can be considered to be 14 nucleotides in length. In some cases, the percent complementarity between the targeting sequence of the targeting segment and the target site of the target nucleic acid is 100% over the seven contiguous 5′-most nucleotides of the target site of the target nucleic acid and as low as 0% or more over the remainder. In such a case, the targeting sequence can be considered to be 20 nucleotides in length.

In some cases, the percent complementarity between the targeting sequence of the targeting segment and the target site of the target nucleic acid is 100% over the 7 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 8 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 9 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 10 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 17 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 18 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 60% or more (e.g., e.g., 65% or more, 70% or more, 75% or more, 80% or more, 85% or more, 90% or more, 95% or more, 97% or more, 98% or more, 99% or more, or 100%) over about 20 contiguous nucleotides.

In some cases, the percent complementarity between the targeting sequence of the targeting segment and the target site of the target nucleic acid is 100% over the 7 contiguous 5′-most nucleotides of the target site of the target nucleic acid and as low as 0% or more over the remainder. In such a case, the targeting sequence can be considered to be 7 nucleotides in length. In some cases, the percent complementarity between the targeting sequence of the targeting segment and the target site of the target nucleic acid is 100% over the 8 contiguous 5′-most nucleotides of the target site of the target nucleic acid and as low as 0% or more over the remainder. In such a case, the targeting sequence can be considered to be 8 nucleotides in length. In some cases, the percent complementarity between the targeting sequence of the targeting segment and the target site of the target nucleic acid is 100% over the 9 contiguous 5′-most nucleotides of the target site of the target nucleic acid and as low as 0% or more over the remainder. In such a case, the targeting sequence can be considered to be 9 nucleotides in length. In some cases, the percent complementarity between the targeting sequence of the targeting segment and the target site of the target nucleic acid is 100% over the 10 contiguous 5′-most nucleotides of the target site of the target nucleic acid and as low as 0% or more over the remainder. In such a case, the targeting sequence can be considered to be 10 nucleotides in length. In some cases, the percent complementarity between the targeting sequence of the targeting segment and the target site of the target nucleic acid is 100% over the 11 contiguous 5′-most nucleotides of the target site of the target nucleic acid and as low as 0% or more over the remainder. In such a case, the targeting sequence can be considered to be 11 nucleotides in length. In some cases, the percent complementarity between the targeting sequence of the targeting segment and the target site of the target nucleic acid is 100% over the 12 contiguous 5′-most nucleotides of the target site of the target nucleic acid and as low as 0% or more over the remainder. In such a case, the targeting sequence can be considered to be 12 nucleotides in length. In some cases, the percent complementarity between the targeting sequence of the targeting segment and the target site of the target nucleic acid is 100% over the 13 contiguous 5′-most nucleotides of the target site of the target nucleic acid and as low as 0% or more over the remainder. In such a case, the targeting sequence can be considered to be 13 nucleotides in length. In some cases, the percent complementarity between the targeting sequence of the targeting segment and the target site of the target nucleic acid is 100% over the 14 contiguous 5′-most nucleotides of the target site of the target nucleic acid and as low as 0% or more over the remainder. In such a case, the targeting sequence can be considered to be 14 nucleotides in length. In some cases, the percent complementarity between the targeting sequence of the targeting segment and the target site of the target nucleic acid is 100% over the 17 contiguous 5′-most nucleotides of the target site of the target nucleic acid and as low as 0% or more over the remainder. In such a case, the targeting sequence can be considered to be 17 nucleotides in length. In some cases, the percent complementarity between the targeting sequence of the targeting segment and the target site of the target nucleic acid is 100% over the 18 contiguous 5′-most nucleotides of the target site of the target nucleic acid and as low as 0% or more over the remainder. In such a case, the targeting sequence can be considered to be 18 nucleotides in length.

### Protein-Binding Segment of a Cas9 Guide RNA

The protein-binding segment of a subject Cas9 guide RNA typically interacts with a Cas9 protein. The Cas9 guide RNA guides the bound Cas9 protein to a specific nucleotide sequence within target nucleic acid via the above mentioned targeting segment. The protein-binding segment of a Cas9 guide RNA typically comprises two stretches of nucleotides that are complementary to one another and hybridize to form a double stranded RNA duplex (dsRNA duplex). Thus, the protein-binding segment can include a dsRNA duplex. In some cases, the protein-binding segment also includes stem loop 1 (the “nexus”) of a Cas9 guide RNA. For example, in some cases, the activator of a Cas9 guide RNA (dgRNA or sgRNA) includes (i) a duplex forming segment that contributes to the dsRNA duplex of the protein-binding segment; and (ii) nucleotides 3′ of the duplex forming segment, e.g., that form stem loop 1 (the “nexus”). For example, in some cases, the protein-binding segment includes stem loop 1 (the “nexus”) of a Cas9 guide RNA. In some cases, the protein-binding segment includes 5 or more nucleotides (nt) (e.g., 6 or more, 7 or more, 8 or more, 9 or more, 10 or more, 11 or more, 12 or more, 15 or more, 20 or more, 30 or more, 40 or more, 50 or more, 60 or more, 70 or more, 75 or more, or 80 or more nt) 3′ of the dsRNA duplex (where 3′ is relative to the duplex-forming segment of the activator sequence).

The dsRNA duplex of the guide RNA (sgRNA or dgRNA) that forms between the activator and targeter is sometimes referred to herein as the “stem loop”. In addition, the activator (activator RNA, tracrRNA) of many naturally existing Cas9 guide RNAs (e.g., S. pygogenes guide RNAs) has 3 stem loops (3 hairpins) that are 3′ of the duplex-forming segment of the activator. The closest stem loop to the duplex-forming segment of the activator (3′ of the duplex forming segment) is called “stem loop 1” (and is also referred to herein as the “nexus”); the next stem loop is called “stem loop 2” (and is also referred to herein as the “hairpin 1”); and the next stem loop is called “stem loop 3” (and is also referred to herein as the “hairpin 2”).

In some cases, a Cas9 guide RNA (sgRNA or dgRNA) (e.g., a full length Cas9 guide RNA) has stem loops 1, 2, and 3. In some cases, an activator (of a Cas9 guide RNA) has stem loop 1, but does not have stem loop 2 and does not have stem loop 3. In some cases, an activator (of a Cas9 guide RNA) has stem loop 1 and stem loop 2, but does not have stem loop 3. In some cases, an activator (of a Cas9 guide RNA) has stem loops 1, 2, and 3.

In some cases, the activator (e.g., tracr sequence) of a Cas9 guide RNA (dgRNA or sgRNA) includes (i) a duplex forming segment that contributes to the dsRNA duplex of the protein-binding segment; and (ii) a stretch of nucleotides (e.g., referred to herein as a 3′ tail) 3′ of the duplex forming segment. In some cases, the additional nucleotides 3′ of the duplex forming segment form stem loop 1. In some cases, the activator (e.g., tracr sequence) of a Cas9 guide RNA (dgRNA or sgRNA) includes (i) a duplex forming segment that contributes to the dsRNA duplex of the protein-binding segment; and (ii) 5 or more nucleotides (e.g., 6 or more, 7 or more, 8 or more, 9 or more, 10 or more, 11 or more, 12 or more, 13 or more, 14 or more, 15 or more, 20 or more, 25 or more, 30 or more, 35 or more, 40 or more, 45 or more, 50 or more, 60 or more, 70 or more, or 75 or more nucleotides) 3′ of the duplex forming segment. In some cases, the activator (activator RNA) of a Cas9 guide RNA (dgRNA or sgRNA) includes (i) a duplex forming segment that contributes to the dsRNA duplex of the protein-binding segment; and (ii) 5 or more nucleotides (e.g., 6 or more, 7 or more, 8 or more, 9 or more, 10 or more, 11 or more, 12 or more, 13 or more, 14 or more, 15 or more, 20 or more, 25 or more, 30 or more, 35 or more, 40 or more, 45 or more, 50 or more, 60 or more, 70 or more, or 75 or more nucleotides) 3′ of the duplex forming segment.

In some cases, the activator (e.g., tracr sequence) of a Cas9 guide RNA (dgRNA or sgRNA) includes (i) a duplex forming segment that contributes to the dsRNA duplex of the protein-binding segment; and (ii) a stretch of nucleotides (e.g., referred to herein as a 3′ tail) 3′ of the duplex forming segment. In some cases, the stretch of nucleotides 3′ of the duplex forming segment has a length in a range of from 5 to 200 nucleotides (nt) (e.g., from 5 to 150 nt, from 5 to 130 nt, from 5 to 120 nt, from 5 to 100 nt, from 5 to 80 nt, from 10 to 200 nt, from 10 to 150 nt, from 10 to 130 nt, from 10 to 120 nt, from 10 to 100 nt, from 10 to 80 nt, from 12 to 200 nt, from 12 to 150 nt, from 12 to 130 nt, from 12 to 120 nt, from 12 to 100 nt, from 12 to 80 nt, from 15 to 200 nt, from 15 to 150 nt, from 15 to 130 nt, from 15 to 120 nt, from 15 to 100 nt, from 15 to 80 nt, from 20 to 200 nt, from 20 to 150 nt, from 20 to 130 nt, from 20 to 120 nt, from 20 to 100 nt, from 20 to 80 nt, from 30 to 200 nt, from 30 to 150 nt, from 30 to 130 nt, from 30 to 120 nt, from 30 to 100 nt, or from 30 to 80 nt). In some cases, the nucleotides of the 3′ tail of an activator RNA are wild type sequences. It will be recognized that a number of different alternative sequences can be used.

Examples of various Cas9 proteins and Cas9 guide RNAs (as well as information regarding requirements related to protospacer adjacent motif (PAM) sequences present in targeted nucleic acids) can be found in the art (see, e.g., Jinek et al. (2012) Science, 337(6096): 816-821; Chylinski et al. (2013) RNA Biol. 10(5):726-737; Ma et al., (2013) Biomed. Res. Int. 2013: 270805; Hou et al. (2013) Proc. Natl. Acad. Sci. USA, 110(39): 15644-15649; Pattanayak et al. (2013) Nat. Biotechnol. 31(9): 839-843; Qi et al. (2013) Cell, 152(5): 1173-1183; Wang et al. (2013) Cell, 153(4): 910-918; Chen et. al. (2013) Nucl. Acids Res. 41(20): e19; Cheng et. al. (2012) Cell Res. 23(10): 1163-1171; Cho et. al. (2013) Genetics, 195(3): 1177-1180; DiCarlo et al. (2013) Nucl. Acids Res. 41(7): 4336-4343; Dickinson et. al. (2013) Nat. Meth. 10(10): 1028-1034; Ebina et. al. (2013) Sci. Rep. 3: 2510; Fujii et. al. (2013) Nucl. Acids Res. 41(20): e187; Hu et. al. (2013) Cell Res. 23(11): 1322-1325; Jiang et. al. (2013) Nucl. Acids Res. 41(20): e188; Larson et. al. (2013) Nat. Protoc. 8(11): 2180-2196; Mali et. al. (2013) Nat. Meth. 10(10): 957-963; Nakayama et. al. (2013) Genesis, 51(12): 835-843; Ran et. al. (2013) Nat. Protoc. 8(11): 2281-2308; Ran et. al. (2013) Cell 154(6): 1380-1389; Walsh et. al. (2013) Proc. Natl. Acad. Sci. USA, 110(39): 15514-15515; Yang et. al. (2013) Cell, 154(6): 1370-1379; Briner et al. (2014) Mol. Cell, 56(2): 333-339; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 2014/0068797; 2014/0170753; 2014/0179006; 2014/0179770; 2014/0186843; 2014/0186919; 2014/0186958; 2014/0189896; 2014/0227787; 2014/0234972; 2014/0242664; 2014/0242699; 2014/0242700; 2014/0242702; 2014/0248702; 2014/0256046; 2014/0273037; 2014/0273226; 2014/0273230; 2014/0273231; 2014/0273232; 2014/0273233; 2014/0273234; 2014/0273235; 2014/0287938; 2014/0295556; 2014/0295557; 2014/0298547; 2014/0304853; 2014/0309487; 2014/0310828; 2014/0310830; 2014/0315985; 2014/0335063; 2014/0335620; 2014/0342456; 2014/0342457; 2014/0342458; 2014/0349400; 2014/0349405; 2014/0356867; 2014/0356956; 2014/0356958; 2014/0356959; 2014/0357523; 2014/0357530; 2014/0364333; and 2014/0377868; all of which are incorporated herein by reference in their entirety.

In certain embodiments, alternative PAM sequences may also be utilized, where a PAM sequence can be NAG as an alternative to NGG (Hsu (2014) supra.) using an S. pyogenes Cas9. Additional PAM sequences may also include those lacking the initial G (see, e.g., Sander and Joung (2014) Nature Biotech 32(4):347). In addition to the S. pyogenes encoded Cas9 PAM sequences, other PAM sequences can be used that are specific for Cas9 proteins from other bacterial sources. For example, the PAM sequences shown below in Table 1 (adapted from Sander and Joung, supra., and Esvelt et al. (2013) Nat. Meth. 10(11): 1116) are specific for these Cas9 proteins:

TABLE 1
Illustrative PAM sequences from various species.

| Species | PAM |
|---|---|
| S. pyogenes | NGG |
| S. pyogenes | NAG |
| S. mutans | NGG |
| S. thermophilius | NGGNG |
| S. thermophilius | NNAAAW |
| S. thermophilius | NNAGAA |
| S. thermophilius | NNNGATT |
| C. jejuni | NNNNACA |
| N. meningitides | NNNNGATT |
| P. multocida | GNNNCNNA |
| F. novicida | NG |

Thus, in certain embodiments, a suitable target sequence for use with a S. pyogenes CRISPR/Cas system can be chosen according to the following guideline: [n17, n18, n19, or n20](G/A)G (SEQ ID NO:1). Alternatively, in certain embodiments, the PAM sequence can follow the guideline G[n17, n18, n19, n20](G/A)G (SEQ ID NO:2). For Cas9 proteins derived from non-S. pyogenes bacteria, the same guidelines may be used where the alternate PAMs are substituted in for the S. pyogenes PAM sequences.

### Guide RNAs Corresponding to Type V and Type VI CRISPR/Cas Endonucleases (e.g., Cpf1 Guide RNA)

A guide RNA that binds to a type V or type VI CRISPR/Cas protein (e.g., Cpf1, C2c1, C2c2, C2c3), and targets the complex to a specific location within a target nucleic acid is referred to herein generally as a “type V or type VI CRISPR/Cas guide RNA”. An example of a more specific term is a “Cpf1 guide RNA.”

In various embodiments, a type V or type VI CRISPR/Cas guide RNA (e.g., cpf1 guide RNA) can have a total length of from 30 nucleotides (nt) to 200 nt, e.g., from 30 nt to 180 nt, from 30 nt to 160 nt, from 30 nt to 150 nt, from 30 nt to 125 nt, from 30 nt to 100 nt, from 30 nt to 90 nt, from 30 nt to 80 nt, from 30 nt to 70 nt, from 30 nt to 60 nt, from 30 nt to 50 nt, from 50 nt to 200 nt, from 50 nt to 180 nt, from 50 nt to 160 nt, from 50 nt to 150 nt, from 50 nt to 125 nt, from 50 nt to 100 nt, from 50 nt to 90 nt, from 50 nt to 80 nt, from 50 nt to 70 nt, from 50 nt to 60 nt, from 70 nt to 200 nt, from 70 nt to 180 nt, from 70 nt to 160 nt, from 70 nt to 150 nt, from 70 nt to 125 nt, from 70 nt to 100 nt, from 70 nt to 90 nt, or from 70 nt to 80 nt). In some cases, a type V or type VI CRISPR/Cas guide RNA (e.g., cpf1 guide RNA) has a total length of at least 30 nt (e.g., at least 40 nt, at least 50 nt, at least 60 nt, at least 70 nt, at least 80 nt, at least 90 nt, at least 100 nt, or at least 120 nt).

In some cases, a Cpf1 guide RNA has a total length of 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, or 50 nt.

Like a Cas9 guide RNA, a type V or type VI CRISPR/Cas guide RNA (e.g., cpf1 guide RNA) can include a target nucleic acid-binding segment and a duplex-forming region (e.g., in some cases formed from two duplex-forming segments, i.e., two stretches of nucleotides that hybridize to one another to form a duplex)

In various embodiments, the target nucleic acid-binding segment of a type V or type VI CRISPR/Cas guide RNA (e.g., cpf1 guide RNA) can have a length of from 15 nt to 30 nt, e.g., 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt. In some cases, the target nucleic acid-binding segment has a length of 23 nt. In some cases, the target nucleic acid-binding segment has a length of 24 nt. In some cases, the target nucleic acid-binding segment has a length of 25 nt.

In certain embodiments, the guide sequence of a type V or type VI CRISPR/Cas guide RNA (e.g., cpf1 guide RNA) can have a length of from 15 nt to 30 nt (e.g., 15 to 25 nt, 15 to 24 nt, 15 to 23 nt, 15 to 22 nt, 15 to 21 nt, 15 to 20 nt, 15 to 19 nt, 15 to 18 nt, 17 to 30 nt, 17 to 25 nt, 17 to 24 nt, 17 to 23 nt, 17 to 22 nt, 17 to 21 nt, 17 to 20 nt, 17 to 19 nt, 17 to 18 nt, 18 to 30 nt, 18 to 25 nt, 18 to 24 nt, 18 to 23 nt, 18 to 22 nt, 18 to 21 nt, 18 to 20 nt, 18 to 19 nt, 19 to 30 nt, 19 to 25 nt, 19 to 24 nt, 19 to 23 nt, 19 to 22 nt, 19 to 21 nt, 19 to 20 nt, 20 to 30 nt, 20 to 25 nt, 20 to 24 nt, 20 to 23 nt, 20 to 22 nt, 20 to 21 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt). In some cases, the guide sequence has a length of 17 nt. In some cases, the guide sequence has a length of 18 nt. In some cases, the guide sequence has a length of 19 nt. In some cases, the guide sequence has a length of 20 nt. In some cases, the guide sequence has a length of 21 nt. In some cases, the guide sequence has a length of 22 nt. In some cases, the guide sequence has a length of 23 nt. In some cases, the guide sequence has a length of 24 nt.

In certain embodiments, the guide sequence of a type V or type VI CRISPR/Cas guide RNA (e.g., cpf1 guide RNA) can have 100% complementarity with a corresponding length of target nucleic acid sequence. The guide sequence can have less than 100% complementarity with a corresponding length of target nucleic acid sequence. For example, the guide sequence of a type V or type VI CRISPR/Cas guide RNA (e.g., cpf1 guide RNA) can have 1, 2, 3, 4, or 5 nucleotides that are not complementary to the target nucleic acid sequence. For example, in some cases, where a guide sequence has a length of 25 nucleotides, and the target nucleic acid sequence has a length of 25 nucleotides, in some cases, the target nucleic acid-binding segment has 100% complementarity to the target nucleic acid sequence. As another example, in some cases, where a guide sequence has a length of 25 nucleotides, and the target nucleic acid sequence has a length of 25 nucleotides, in some cases, the target nucleic acid-binding segment has 1 non-complementary nucleotide and 24 complementary nucleotides with the target nucleic acid sequence. As another example, in some cases, where a guide sequence has a length of 25 nucleotides, and the target nucleic acid sequence has a length of 25 nucleotides, in some cases, the target nucleic acid-binding segment has 2 non-complementary nucleotides and 23 complementary nucleotides with the target nucleic acid sequence.

In certain embodiments, the duplex-forming segment of a type V or type VI CRISPR/Cas guide RNA (e.g., cpf1 guide RNA) (e.g., of a targeter RNA or an activator RNA) can have a length of from 15 nt to 25 nt (e.g., 15 nt, 16 nt, 17 nt, 18 nt, 19 nt, 20 nt, 21 nt, 22 nt, 23 nt, 24 nt, or 25 nt).

The RNA duplex of a type V or type VI CRISPR/Cas guide RNA (e.g., cpf1 guide RNA) can have a length of from 5 base pairs (bp) to 40 bp (e.g., from 5 to 35 bp, 5 to 30 bp, 5 to 25 bp, 5 to 20 bp, 5 to 15 bp, 5-12 bp, 5-10 bp, 5-8 bp, 6 to 40 bp, 6 to 35 bp, 6 to 30 bp, 6 to 25 bp, 6 to 20 bp, 6 to 15 bp, 6 to 12 bp, 6 to 10 bp, 6 to 8 bp, 7 to 40 bp, 7 to 35 bp, 7 to 30 bp, 7 to 25 bp, 7 to 20 bp, 7 to 15 bp, 7 to 12 bp, 7 to 10 bp, 8 to 40 bp, 8 to 35 bp, 8 to 30 bp, 8 to 25 bp, 8 to 20 bp, 8 to 15 bp, 8 to 12 bp, 8 to 10 bp, 9 to 40 bp, 9 to 35 bp, 9 to 30 bp, 9 to 25 bp, 9 to 20 bp, 9 to 15 bp, 9 to 12 bp, 9 to 10 bp, 10 to 40 bp, 10 to 35 bp, 10 to 30 bp, 10 to 25 bp, 10 to 20 bp, 10 to 15 bp, or 10 to 12 bp).

As an illustrative, but non-limiting example, a duplex-forming segment of a Cpf1 guide RNA can comprise a nucleotide sequence selected from (5′ to 3′): AAUUUCUACUGUUGUAGAU (SEQ ID NO:3), AAUUUCUGCUGUUGCAGAU (SEQ ID NO:4), AAUUUCCACUGUUGUGGAU (SEQ ID NO:5), AAUUCCUACUGUUGUAGGU (SEQ ID NO:6), AAUUUCUACUAUUGUAGAU (SEQ ID NO:7), AAUUUCUACUGCUGUAGAU (SEQ ID NO:8), AAUUUCUACUUUGUAGAU (SEQ ID NO:9), AAUUUCUACUUGUAGAU (SEQ ID NO:10), and the like. The guide sequence can then follow (5′ to 3′) the duplex forming segment.

Examples and guidance related to type V or type VI CRISPR/Cas endonucleases and guide RNAs (as well as information regarding requirements related to protospacer adjacent motif (PAM) sequences present in targeted nucleic acids) can be found in the art (see, e.g., Zetsche et al. (2015) Cell, 163(3): 759-771; Makarova et al. (2015) Nat. Rev. Microbiol. 13(11): 722-736; Shmakov et al. (2015) Mol. Cell, 60(3): 385-397; and the like).

Using the teachings provided herein, methods utilizing different CRISPR/Cas endonucleases and gRNAs to knock down or to knock out the CRYAB gene will be available to one of skill in the art.

### Zinc Finger Endonucleases.

In certain embodiments, the targeting endonuclease can be a zinc finger nuclease (ZFN). Typically, a zinc finger nuclease comprises a DNA binding domain (e.g., zinc finger) and a cleavage domain (e.g., nuclease), both of which are described below.

### Zinc Finger Binding Domain.

Zinc finger binding domains may be engineered to recognize and bind to any nucleic acid sequence of choice (see, e.g., Beerli et al. (2002) Nat. Biotechnol. 20: 135-141; Pabo et al. (2001) Ann. Rev. Biochem. 70: 313-340; Isalan et al. (2001) Nat. Biotechnol. 19: 656-660; Segal et al. (2001) Curr. Opin. Biotechnol. 12: 632-637; Choo et al. (2000) Curr. Opin. Struct. Biol. 10: 411-416; Zhang et al. (2000) J. Biol. Chem. 275(43): 33850-33860; Doyon et al. (2008) Nat. Biotechnol. 26: 702-708; and Santiago et al. (2008) Proc. Natl. Acad. Sci. USA, 105: 5809-5814). An engineered zinc finger binding domain can have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence (see, e.g., U.S. Pat. Nos. 6,453,242 and 6,534,261, and the like). As an example, the algorithm described in U.S. Pat. No. 6,453,242 may be used to design a zinc finger binding domain to target a preselected sequence. Alternative methods, such as rational design using a nondegenerate recognition code table can also be used to design a zinc finger binding domain to target a specific sequence (see, e.g., Sera et al. (2002) Biochemistry 41: 7074-7081; and the like). Publically available web-based tools for identifying target sites in DNA sequences and designing zinc finger binding domains are found, inter alia, at www.zincfingertools.org and zifit.partners.org/ZiFiT/(see also Mandell et al. (2006) Nucl. Acida Res. 34: W516-W523; Sander et al. (2007) Nucl. Acida Res. 35: W599-W605; and the like).

A zinc finger binding domain may be designed to recognize and bind a DNA sequence ranging from about 3 nucleotides to about 21 nucleotides in length, for example, from about 9 to about 18 nucleotides in length. Each zinc finger recognition region (i.e., zinc finger) typically recognizes and binds three nucleotides. In certain embodiments, the zinc finger binding domains of suitable targeted zinc finger nucleases comprise at least three zinc finger recognition regions (i.e., zinc fingers). The zinc finger binding domain, however, may comprise four, or five, or six, or more zinc finger recognition regions. A zinc finger binding domain may be designed to bind to any suitable target DNA sequence (see, e.g., U.S. Pat. Nos. 6,607,882; 6,534,261, 6,453,242, and the like.

Illustrative methods of selecting a zinc finger recognition region include, but are not limited to phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237y. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 2005/0064474 and 2006/0188987. Zinc finger recognition regions and/or multi-fingered zinc finger proteins may be linked together using suitable linker sequences, including for example, linkers of five or more amino acids in length (see, e.g., U.S. Pat. Nos. 6,479,626; 6,903,185; and 7,153,949) for non-limiting examples of linker sequences of six or more amino acids in length.

### Cleavage Domain.

A zinc finger nuclease also typically includes a cleavage domain. The cleavage domain portion of the zinc finger nuclease may be obtained from any endonuclease or exonuclease. Non-limiting examples of endonucleases from which a cleavage domain may be derived include, but are not limited to, restriction endonucleases and homing endonucleases (see, e.g., New England Biolabs catalog (www.neb.com); Belfort et al. (1997) Nucleic Acids Res. 25:3379-3388; and the like). Additional enzymes that cleave DNA are known (e.g., S1 Nuclease; mung bean nuclease; pancreatic DNase I; micrococcal nuclease; yeast HO endonuclease). In certain embodiments, one or more of these enzymes (or functional fragments thereof) may be used as a source of cleavage domains.

In certain embodiments, a cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease can comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

In various embodiments, when two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs can intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other (see, e.g., U.S. Pat. Nos. 5,356,802; 5,436,150, and 5,487,994; Li et al. (1992) Proc. Natl. Acad. Sci. USA, 89: 4275-4279; Li et al. (1993)Proc. Natl. Acad. Sci. USA, 90: 2764-2768. Thus, a zinc finger nuclease can comprise the cleavage domain from at least one Type IIS restriction enzyme and one or more zinc finger binding domains, which may or may not be engineered. Illustrative type IIS restriction enzymes are described for example in International Patent Publication No: WO 07/014,275. Additional restriction enzymes also contain separable binding and cleavage domains, and these also are contemplated by the present disclosure (see, e.g., Roberts et al. (2003) Nucleic Acids Res. 31:418-420.

An illustrative Type IIS restriction enzyme, whose cleavage domain is separable from the binding domain, is FokI. This particular enzyme is active as a dimer (Bitinaite et al. (1998) Proc. Natl. Acad. Sci. USA 95: 10, 570-10, 575). Accordingly, for the purposes of the present disclosure, the portion of the FokI enzyme used in a zinc finger nuclease is considered a cleavage monomer. Thus, for targeted double-stranded cleavage using a FokI cleavage domain, two zinc finger nucleases, each comprising a FokI cleavage monomer, may be used to reconstitute an active enzyme dimer. Alternatively, a single polypeptide molecule containing a zinc finger binding domain and two FokI cleavage monomers can also be used.

In certain embodiments, the cleavage domain may comprise one or more engineered cleavage monomers that minimize or prevent homodimerization, as described, for example, in U.S. Patent Publication Nos. 2005/0064474, 2006/0188987, 2008/0131962, and the like. By way of non-limiting example, amino acid residues at positions 446, 447, 479, 483, 484, 486, 487, 490, 491, 496, 498, 499, 500, 531, 534, 537, and 538 of FokI are all targets for influencing dimerization of the FokI cleavage half-domains. Illustrative engineered cleavage monomers of FokI that form obligate heterodimers include a pair in which a first cleavage monomer includes mutations at amino acid residue positions 490 and 538 of FokI and a second cleavage monomer that includes mutations at amino-acid residue positions 486 and 499 (see, e.g., Miller et al. (2007) Nat. Biotechnol. 25: 778-785; Szczpek et al. (2007) Nat. Biotechnol. 25: 786-793). For example, the Glu (E) at position 490 may be changed to Lys (K) and the lie (I) at position 538 may be changed to K in one domain (E490K, 1538K), and the Gin (Q) at position 486 may be changed to E and the I at position 499 may be changed to Leu (L) in another cleavage domain (Q486E, 1499L). In other aspects, modified FokI cleavage domains can include three amino acid changes (see, e.g., Doyon et al. (2011) Nat. Methods, 8: 74-81). For example, one modified FokI domain (which is termed ELD) can comprise Q486E, 1499L, N496D mutations and the other modified FokI domain (which is termed KKR) can comprise E490K, 1538K, H537R mutations.

### Additional Domains.

In certain embodiments, the zinc finger nuclease further comprises at least one nuclear localization signal or sequence (NLS). A NLS is an amino acid sequence that facilitates transport of the zinc finger nuclease protein into the nucleus of eukaryotic cells. In general, an NLS comprise a stretch of basic amino acids. Nuclear localization signals are known in the art (see, e.g., Makkerh et al. (1996) Curr. Biol. 6: 1025-1027; Lange et al. (2007) J. Biol. Chem. 282: 5101-5105). For example, in one embodiment, the NLS can be a monopartite sequence, such as PKKKRKV (SEQ ID NO:11) or PKKKRRV (SEQ ID NO:12). In another embodiment, the NLS can be a bipartite sequence. In still another embodiment, the NLS can be KRPAATKKAGQAKKKK (SEQ ID NO:13). In various embodiments, the NLS can be located at the N-terminus, the C-terminus, or in an internal location of the zinc finger nuclease.

Although not required in the methods described herein, in certain embodiments, the zinc finger nuclease can also comprise at least one cell-penetrating domain. In one embodiment, the cell-penetrating domain can be a cell-penetrating peptide sequence derived from the HIV-1 TAT protein. As an example, the TAT cell-penetrating sequence can be GRKKRRQRRRPPQPKKKRKV (SEQ ID NO:14). In another embodiment, the cell-penetrating domain can be TLM (PLSSIFSRIGDPPKKKRKV, SEQ ID NO:15), a cell-penetrating peptide sequence derived from the human hepatitis B virus. In still another embodiment, the cell-penetrating domain can be MPG (GALFLGWLGAAGSTMGAPKKKRKV, SEQ ID NO:16) or GALFLGFLGAAGSTMGAWSQPKKKRKV, SEQ ID NO:17). In an additional embodiment, the cell-penetrating domain can be Pep-1 (KETWWETWWTEWSQPKKKRKV, SEQ ID NO:18), VP22, a cell penetrating peptide from Herpes simplex virus, or a polyarginine peptide sequence. In various embodiments, the cell-penetrating domain can be located at the N-terminus, the C-terminus, or in an internal location of the protein.

Using the teaching provided herein numerous constructs utilizing CRISPR, TALEN, or zinc finger endonucleases to knock down or to knock out CRYAB will be available to one of skill in the art.

### Antibody CRYAB Inhibitors

In certain embodiments, the inhibitors of CRYAB expression and/or aggregation can comprise antibodies, including, without limitation, monoclonal antibodies, polyclonal antibodies, recombinant antibodies, chimeric antibodies, naturally occurring antibodies, or specific antigen binding and/or functional domains, motifs, or fragments thereof. In various embodiments, the antibodies can include human antibodies, non-human animal antibodies from any animal species (e.g., mouse, rat, rabbit, chicken, dog, goat, camelids [dromedaries, camels, llamas and alpacas], and monkey), and/or humanized antibodies.

Techniques for the production of antibodies are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999.

In certain embodiments, the antibodies of the invention are directed to one or more epitopes within the expressed CRYAB protein (see, e.g., UniProtKB—P02511, and SEQ ID NO:19).

```
(SEQ ID NO: 19)
                10 20 30 40
MDIAIHHPWI RRPFFPFHSP SRLFDQFFGE HLLESDLFPT
                50 60 70 80
STSLSPFYLR PPSFLRAPSW FDTGLSEMRL EKDRFSVNLD
                90 100 110 120
VKHFSPEELK VKVLGDVIEV HGKHEERQDE HGFISREFHR
              130 140 150 160
KYRIPADVDP LTITSSLSSD GVLTVNGPRK QVSGPERTIP
              170
ITREEKPAVT AAPKK
```

In certain embodiments, the antibody binds to an epitope comprising at least 15 contiguous amino acids, or at least 20 contiguous amino acids, or at least 25 contiguous amino acids, or at least 30 contiguous amino acids, or at least 35contiguous amino acids of the CRYAB protein sequence (e.g., SEQ ID NO:19)).

In certain embodiments, antibodies used to inhibit CRYAB expression and/or aggregation include, but are not limited to, bispecific antibodies, that bind to two different antigens, e.g., binds to two different epitopes on the CRYAB protein. In certain embodiments, the bispecific antibodies are diabodies, which are a class of small bivalent and bispecific antibody fragments that can be expressed in bacteria (e.g. E. coli), yeast (e.g. Pichia pastoris), and cells from higher eukaryotic organisms in functional form and with high yields (up to 1 g/L). Diabodies comprise a heavy (VH) chain variable domain connected to a light chain variable domain (VL) on the same polypeptide chain (VH-VL) connected by a peptide linker that is too short to allow pairing between the two domains on the same chain. This forces pairing with the complementary domains of another chain and promotes the assembly of a dimeric molecule with two functional antigen binding sites.

To construct bispecific diabodies, the variable domains derived from antibody A and antibody B can be fused to create the two chains VHA-VLB, VHB-VLA. Each chain is inactive in binding to antigen, but recreates the functional antigen binding sites of antibodies A and B on pairing with the other chain.

Anti-CRYAB antibodies useful in the methods described herein can be generated using any antibody producing methods, now known or later developed in the art. For example, phage display methods can be used. In phage display methods, functional antibody domains are displayed on the surface of phage particles that carry the nucleic acid sequences encoding them. In one illustrative embodiment, such phage can be utilized to display antigen binding domains expressed from a repertoire or combinatorial antibody library (e.g., human or murine). In particular, DNA sequences encoding V_{H} and V_{L} domains can be amplified from animal cDNA libraries (e.g., human or murine cDNA libraries of lymphoid tissues). The DNA encoding the V_{H} and V_{L} domains can be recombined together with an scFv linker by PCR and cloned into a phagemid vector. The vector can electroporated in E. coli and the E. coli is infected with helper phage. Phage used in these methods are typically filamentous phage including fd and M13 binding domains expressed from phage with Fab, Fv or disulfide stabilized Fv antibody domains recombinantly fused to either the phage gene III or gene VIII protein. Phage expressing an antigen binding domain that binds to or-portions thereof can be selected or identified with antigen e.g., using labeled antigen or antigen bound or captured to a solid surface or bead. Alternatively it is also possible to produce the recombinant bispecific antibody constructs by hybridoma fusion.

It is also noted that a number of anti-CRYAB antibodies are known to those of skill in the art and are commercially available. In certain embodiments, these antibodies can be used as is or the sequences of these antibodies (e.g., the complementarity determining regions (CDRs), or the V_{H} and V_{L} domains can be used to produce chimeric or fully human antibodies or to engineer other antibody fors (e.g., Fab, scFv, etc.).

In various embodiments, the antibodies are delivered into the cytoplasm of senescent cells. In certain embodiments, this can be accomplished using, e.g., internalizing antibodies, single chain (scFv) antibodies, antibody fragments, cameloid antibodies, and the like. Alternatively, enhanced and/or targeted delivery of anti-CYRAB antibodies can be achieved by using bispecific antibodies with the second antigen being specific to senescent cells.

As noted above, in some embodiments, the anti-CRYAB antibodies will need to be able to function within the target cell. Specifically, in certain embodiments, the antibodies must be able to enter the target cell cytoplasm and/or organelles and must also be soluble and capable of specifically binding to CRYAB protein within an intracellular environment. These intracellular antibodies can be introduced into cells by a variety of methods, including for example and not limitation, various cell penetrating compositions (see, e.g., WO 2013/138795, WO 2015/031837, U.S. Pat. No. 7,157,087), and vesicles comprising liposomes and/or block copolymers (see, e.g., WO 2009/138473). Stability of intracellular antibodies can be improved by, for example and not limitation, the methods and compositions taught in U.S. Pat. No. 7,608,453, WO 2004046186, U.S. Pat. Nos. 8,853,362, and 7,258,986.

Therapeutic and/or Prophylactic Methods

In various embodiments, methods are provided for selectively killing one or more senescent cells in a sample (e.g., in a biological sample), where the method involves contacting the sample with an effective amount of one or more agents that inhibit expression and/or aggregation of a CRYAB protein (e.g., as described herein).

In certain embodiments, methods are provided for selectively killing one or more senescent cells in a subject in need thereof, where the method involves contacting the sample with an effective amount of one or more agents that inhibit expression and/or aggregation of a CRYAB protein (e.g., as described herein). In certain embodiments the subject in need thereof is a subject with an age-related disorder. In certain embodiments the subject in need thereof has a pathology characterized by production of senescent cells and/or an inflammatory response.

By selectively killing one or more senescent cells is meant that the agent that inhibits CRYAB expression and/or aggregation (CRYAB inhibitor), does not appreciably kill non-senescent cells at the same concentration. Accordingly, in certain embodiments, the median lethal dose or LD50 of the CRYAB inhibitor in non-senescent cells may be about 5 to about 500, or about 5 to about 400, or about 5 to about 300, or about 5 to about 300, or about 5 to about 200, or about 5 to about 100, or about 5 to about 90, or about 5 to about 80, or about 5 to about 70, or about 5 to about 60, or about 5 to about 50 times higher than the LD50 of the CRYAB inhibitor in senescent cells. As used herein, the LD50 is the concentration of CRYAB inhibitor required to kill half the cells in the cell sample. For example, the LD50 of the CRYAB inhibitor in non-senescent cells may be greater than about 5, about 6, about 7, about 8, about 9 or about 10 times higher than the LD50 of the CRYAB inhibitor in senescent cells. In certain embodiments, the LD50 of the CRYAB inhibitor in non-senescent cells may be greater than about 10, about 15, about 20, about 25, about 30, about 35, about 40, about 45, or about 50 times higher than the LD50 of the CRYAB inhibitor in senescent cells. In certain embodiments, the LD50 of the CRYAB inhibitor in non-senescent cells may be greater than about 50, about 100, about 200, about 300, about 400, about 500 times higher than the LD50 of the CRYAB inhibitor in senescent cells. In certain embodiments, the LD50 of the CRYAB inhibitor in non-senescent cells may be greater than 50 times higher than the LD50 of the CRYAB inhibitor in senescent cells. In one illustrative embodiment, the LD50 of the CRYAB inhibitor in non-senescent cells is greater than 10 times higher than the LD50 of the CRYAB inhibitor in senescent cells. In another illustrative embodiment, the LD50 of the CRYAB inhibitor in non-senescent cells is greater than 20 times higher than the LD50 of the CRYAB inhibitor in senescent cells.

The progression from an actively dividing cell to a metabolically active, non-dividing cell is termed “senescence” or “cellular senescence.” As used herein, the terms “senescence” and “cellular senescence” may be used interchangeably. The term “senescence” also refers to the state into which cells enter after multiple rounds of division and, as a result of cellular pathways, future cell division is prevented from occurring even though the cell remains metabolically active. Senescent cells may differ from their pre-senescent counterparts in one or more of the following ways: 1) they have arrested growth and cannot be stimulated to reenter the cell cycle by physiological mitogens; 2) they become resistant to apoptotic cell death; and/or 3) they acquire altered differentiated functions.

In contrast to cancer cells that grow and divide uncontrollably, the ability of most differentiated eukaryotic cells to proliferate is finite. Stated another way, normal cells have an intrinsically determined limit to the number of cell divisions through which they can proceed. This phenomenon has been termed “replicative cellular senescence” and is an intrinsic anticancer mechanism that limits a cell's proliferative ability, thereby preventing neoplastic progression. Another form of senescence is “stress-induced cellular senescence” (sometimes inaccurately termed premature senescence). Stress-induced cellular senescence, like replicative cellular senescence, is a terminal fate of mitotic cells, characterized by permanent cell cycle arrest. Unlike replicative cellular senescence, however, stress-induced cellular senescence does not require telomere deterioration and can be induced by a variety of stressors including, but not limited to, ultraviolet light, reactive oxygen species, certain chemotherapeutics, environmental toxins, cigarette smoking, ionizing radiation, distortion of chromatin structure, excessive mitogenic signaling, and oncogenic mutations. Still another form of senescence is therapy-induced senescence (TIS) which refers to the phenomenon of a subset of cells (e.g., neoplastic cells such as tumor cells) being forced into a senescent state by therapeutic agents. TIS is known to develop because of certain treatments, including radiotherapy and certain chemotherapies such as cancer medications and HIV medications.

The number of senescent cells in various organs and tissues of a subject is known to increase with age. The accumulation of senescent cells may drive various aspects of the deterioration that underlies aging and age-related diseases. For example, the accumulation of senescent cells in aged tissue may contribute to age-associated tissue dysfunction, reduced regenerative capacity, and disease. In this context, senescence is considered deleterious because it contributes to decrements in tissue renewal and function. As a non-limiting example, an aged tissue may lack the ability to respond to stress when proliferation is required thereby resulting in the reduced fitness seen with aging

### Cellular Targets—Senescent Cells

The method described herein involves the specific or preferential killing of senescent cells (e.g., cells expressing a SASP) in a clinically significant or biologically significant manner (e.g., non-senescent cells are not killed or where killed the cell death produces no pathological symptoms). As discussed in detail herein, the one or more senolytic agents (CRYAB inhibitor(s) is used in an amount and for a time sufficient that selectively kills established senescent cells but is insufficient to kill (destroy, cause the death of) a non-senescent cell in a clinically significant or biologically significant manner. The agents may selectively kill one or more types of senescent cells (e.g., senescent preadipocytes, senescent endothelial cells, senescent fibroblasts, senescent fibro adipogenic progenitors, senescent skeletal muscle satellite cells, senescent neurons, senescent epithelial cells, senescent mesenchymal cells, senescent smooth muscle cells, senescent macrophages, or senescent chondrocytes).

A senescent cell may exhibit any one or more of the following seven characteristics. (1) Senescence growth arrest is essentially permanent and cannot be reversed by known physiological stimuli. (2) Senescent cells increase in size, sometimes enlarging more than twofold relative to the size of non-senescent counterparts. (3) Senescent cells express a senescence-associated β-galactosidase (SA-β-gal), which partly reflects the increase in lysosomal mass. (4) Most senescent cells express p16INK4a, which is not commonly expressed by quiescent or terminally differentiated cells. (5) Cells that senesce with persistent DNA damage response (DDR) signaling harbor persistent nuclear foci, termed DNA segments with chromatin alterations reinforcing senescence (DNA-SCARS). These foci contain activated DDR proteins and are distinguishable from transient damage foci. DNA-SCARS include dysfunctional telomeres or telomere dysfunction-induced foci (TIF). (6) Senescent cells express and may secrete molecules associated with senescence, which in certain instances may be observed in the presence of persistent DDR signaling, which in certain instances may be dependent on persistent DDR signaling for their expression. (7) The nuclei of senescent cells lose structural proteins such as Lamin B1 or chromatin-associated proteins such as histones and HMGB1. See, e.g., Freund et al., Mol. Biol. Cell 23:2066-75 (2012); Davalos et al., J. Cell Biol. 201:613-29 (2013); Ivanov et al., J. Cell Biol. DOI: 10.1083/jcb.201212110, page 1-15; published online Jul. 1, 2013; Funayama et al., J. Cell Biol. 175:869-80 (2006)).

Senescent cells and senescent cell associated molecules can be detected by techniques and procedures described in the art. For example, the presence of senescent cells in tissues can be analyzed by histochemistry or immunohistochemistry techniques that detect the senescence marker, SA-beta galactosidase (SA-βgal) (see, e.g., Dimri et al. (1995) Proc. Natl. Acad. Sci. USA, 92: 9363-9367). The presence of the senescent cell-associated polypeptide p16INK4a can be determined by any one of numerous immunochemistry methods practiced in the art, such as immunoblotting analysis. Expression of p16INK4a mRNA in a cell can be measured by a variety of techniques practiced in the art including quantitative PCR. The presence and level of senescent cell associated polypeptides (e.g., polypeptides of the SASP) can be determined by using automated and high throughput assays, such as an automated Luminex array assay described in the art (see, e.g., Coppe et al. (2008) PLoS Biol. 6: 2853-68 (2008)).

The presence of senescent cells can also be determined by detection of senescent cell-associated molecules, which include growth factors, proteases, cytokines (e.g., inflammatory cytokines), chemokines, cell-related metabolites, reactive oxygen species (e.g., H₂O₂), and other molecules that stimulate inflammation and/or other biological effects or reactions that may promote or exacerbate the underlying disease of the subject. Senescent cell-associated molecules include those that are described in the art as comprising the senescence-associated secretory phenotype (SASP, i.e., which includes secreted factors which may make up the pro-inflammatory phenotype of a senescent cell), senescent-messaging secretome, and DNA damage secretory program (DDSP). These groupings of senescent cell associated molecules, as described in the art, contain molecules in common and are not intended to describe three separate distinct groupings of molecules. Senescent cell-associated molecules include certain expressed and secreted growth factors, proteases, cytokines, and other factors that may have potent autocrine and paracrine activities (see, e.g., Coppe et al., supra; Coppe et al. J. Biol. Chem. 281:29568-74 (2006); Coppe et al. PLoS One 5:39188 (2010); Krtolica et al. Proc. Natl. Acad. Sci. U.S.A. 98:12072-77 (2001); Parrinello et al. (2005) J. Cell Sci. 118:485-496). Extracellular matrix (ECM) associated factors include inflammatory proteins and mediators of ECM remodeling and which are strongly induced in senescent cells (see, e.g., Kuilman et al. (2009) Nature Rev. 9: 81-94). Other senescent cell-associated molecules include extracellular polypeptides (proteins) described collectively as the DNA damage secretory program (DDSP) (see, e.g., Sun et al. (2012) Nature Med. 18: 1359-1368). Senescent cell-associated proteins also include cell surface proteins (or receptors) that are expressed on senescent cells, which include proteins that are present at a detectably lower amount or are not present on the cell surface of a non-senescent cell.

Senescence cell-associated molecules include secreted factors that may make up the pro-inflammatory phenotype of a senescent cell (e.g., SASP). These factors include, without limitation, GM-CSF, GROα, GROα,β, γ, IGFBP-7, IL-1α, IL-6, IL-7, IL-8, MCP-1, MCP-2, MIP-1α, MMP-1, MMP-10, MMP-3, Amphiregulin, ENA-78, Eotaxin-3, GCP-2, GITR, HGF, ICAM-1, IGFBP-2, IGFBP-4, IGFBP-5, IGFBP-6, IL-13, IL-1β, MCP-4, MIF, MIP-3α, MMP-12, MMP-13, MMP-14, NAP2, Oncostatin M, osteoprotegerin, PIGF, RANTES, sgp130, TIMP-2, TRAIL-R3, Acrp30, angiogenin, Ax1, bFGF, BLC, BTC, CTACK, EGF-R, Fas, FGF-7, G-CSF, GDNF, HCC-4, 1-309, IFN-γ, IGFBP-1, IGFBP-3, IL-1 R1, IL-11, IL-15, IL-2R-α, IL-6 R, I-TAC, Leptin, LIF, MMP-2, MSP-a, PAI-1, PAI-2, PDGF-BB, SCF, SDF-1, sTNF RI, sTNF RII, Thrombopoietin, TIMP-1, tPA, uPA, uPAR, VEGF, MCP-3, IGF-1, TGF-β3, MIP-1-delta, IL-4, FGF-7, PDGF-BB, IL-16, BMP-4, MDC, MCP-4, IL-10, TIMP-1, Fit-3 Ligand, ICAM-1, Ax1, CNTF, INF-γ, EGF, BMP-6. Additional identified factors, which include those sometimes referred to in the art as senescence messaging secretome (SMS) factors, some of which are included in the listing of SASP polypeptides, include without limitation, IGF1, IGF2, and IGF2R, IGFBP3, IDFBP5, IGFBP7, PA11, TGF-β, WNT2, IL-1α, IL-6, IL-8, and CXCR2-binding chemokines. Cell-associated molecules also include without limitation the factors described in Sun et al., Nature Medicine, supra, and include, including, for example, products of the genes, MMP1, WNT16B, SFRP2, MMP12, SPINK1, MMP10, ENPP5, EREG, BMP6, ANGPTL4, CSGALNACT, CCL26, AREG, ANGPT1, CCK, THBD, CXCL14, NOV, GAL, NPPC, FAM150B, CST1, GDNF, MUCL1, NPTX2, TMEM155, EDN1, PSG9, ADAMTS3, CD24, PPBP, CXCL3, MMP3, CST2, PSG8, PCOLCE2, PSG7, TNFSF15, C17orf67, CALCA, FGF18, IL8, BMP2, MATN3, TFP1, SERPINI 1, TNFFRSF25, and IL23A. Senescent cell-associated proteins also include cell surface proteins (or receptors) that are expressed on senescent cells, which include proteins that are present at a detectably lower amount or are not present on the cell surface of a non-senescent cell.

In certain embodiments, senolytic agents described herein (CRYAB inhibitors) that selectively kill at least senescent preadipocytes may be useful for treatment of diabetes (particularly type 2 diabetes), metabolic syndrome, or obesity. In other embodiments, senolytic agents are capable of selectively killing at least senescent endothelial cells, senescent smooth muscle cells, and/or senescent macrophages. Such CRYAB inhibitors may be useful for treatment of a cardiovascular disease (e.g., atherosclerosis). In other particular embodiments, the CRYAB inhibitors are capable of selectively killing at least senescent fibroblasts. In still another embodiment, the CRYAB inhibitors agents may selectively kill at least senescent neurons, including astrocytes. In still another embodiment, the senolytic agents may kill at least senescent retinal pigmented epithelial cells or other senescent epithelial cells (e.g., pulmonary senescent epithelial cells or senescent kidney (renal) epithelial cells). Selective killing of at least senescent pulmonary epithelial cells may be useful for treating pulmonary diseases, such as chronic obstructive pulmonary disease or idiopathic pulmonary fibrosis. In yet other embodiments, the CRYAB inhibitors may selectively kill at least senescent immune cells (such as senescent macrophages). In still another embodiment, the CRYAB inhibitors may kill at least senescent chondrocytes, which may be useful for treatment of an inflammatory disorder, such as osteoarthritis. In still another embodiment, the CRYAB inhibitors may kill at lease senescent fibro adipogenic progenitors or skeletal muscle satellite cells, which may be useful for treatment of skeletal muscle disorders such as sarcopenia and chemotherapy-related fatigue/wasting/physical dysfunction.

Senescent cells that are the targets of the methods described herein may be senescent due to replicative cellular senescence, stress-induced cellular senescence or therapy-induced senescence. A senescent cell that is senescent due to stress may be induced by, but not limited to one or more of, ultraviolet light, reactive oxygen species, chemotherapeutics, environmental toxin, cigarette smoking, ionizing radiation, distortion of chromatin structure, excessive mitogenic signaling, and oncogenic mutations. In a specific embodiment, premature cellular senescence may be induced by ionizing radiation (IR). A senescent cell that is therapy-induced senescent may have been exposed to DNA-damaging therapy.

Non-limiting examples of senescent cells may include, but are not limited to, mammary epithelial cells, keratinocytes, cardiac myocytes, chondrocytes, endothelial cells (large vessels), endothelial cells (microvascular), epithelial cells, fibroblasts, follicle dermal papilla cells, hepatocytes, melanocytes, osteoblasts, preadipocytes, primary cells of the immune system, skeletal muscle cells, fibro adipogenic progenitors, skeletal muscle satellite cells, smooth muscle cells, adipocytes, neurons, glial cells, contractile cells, exocrine secretory epithelial cells, extracellular matrix cells, hormone secreting cells, keratinizing epithelial cells, islet cells, lens cells, mesenchymal stem cells, pancreatic acinar cells, paneth cells of the small intestine, primary cells of hemopoietic linage, primary cells of the nervous system, sense organ and peripheral neuron supporting cells, wet stratified barrier epithelial cells and the like.

In certain embodiments, senescent cells that are targets in the methods described herein may be found in renewable tissues, including the vasculature, hematopoietic system, epithelial organs and the stroma. The senescent cells may also be found at sites of aging or chronic age-related pathology, such as osteoarthritis and atherosclerosis. Further, the senescent cell may be associated with benign dysplastic or preneoplastic lesions and benign prostatic hyperplasia. In certain embodiments, the target senescent cell(s) may be found in normal and tumor tissues following DNA-damaging therapy. In a specific embodiment, a senescent cell may be found at a site of aging or age-related pathology.

### Prevention or Treatment of Therapy Induced Senescent Cells

In various embodiments, methods are also provided for killing therapy-induced senescent cells. The methods comprise administering a composition comprising a therapeutically effective amount of a CRYAB inhibitor to a subject that has received DNA-damaging therapy or prophylactically to a subject that is about to undergo a DNA-damaging therapy, or concurrently with a DNA damaging therapy and killing therapy induced-senescent cells in normal and/or tumor tissues following DNA-damaging therapy.

Based on the observation that ionizing radiation and various chemotherapeutic agents elicit a marked senescence response in vivo, therapy-induced senescent cells may be a cause of long-term ramifications after DNA-damaging therapy, such as cancer therapy. As such, the systemic accumulation of therapy-induced senescent cells may drive accelerated physical decline in cancer survivors. Accelerated physical decline may also be referred to as accelerated aging. Accordingly, once neoplastic cells are removed or eliminated by systemic radiation or chemotherapy, senescence may be triggered in a variety of other organs, leading to long-term ramifications for the patient. Long-term ramifications may include reduced quality of life predisposing the subject to disabilities and comorbidities. For example, a subject that has received DNA-damaging therapy may experience a disproportionate decline in physical function, such as inability to climb stairs, or to reach up to put things onto shelves and/or increased functional disabilities such as difficulty eating, dressing and maintaining adequate hygiene. Additionally, late effects of ionizing radiation may include long-term bone marrow injury and/or lung fibrosis. Long-term bone marrow injury can promote hypoplastic anemia and/or myelodysplastic syndrome or leukemia. Additionally, it has been demonstrated that following ionizing radiation, senescent cells in lung, muscle and brain are greatly increased. These long-term ramifications provide a link between accelerated aging and cancer treatment. Accordingly, in various embodiments, administration of one or more CRYAB inhibitors is contemplated, e.g., as an adjuvant (adjunct) therapy in the treatment of a cancer. Illustrative cancers in which administration of one or more CRYAB inhibitors may provide an appropriate adjuvant therapy include, but are not limited to a cancer selected from the group consisting of acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Seźary syndrome), Hodgkin, non-Hodgkin, primary central nervous system (CNS)), macroglobulinemia, Waldenstram, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, chronic myelogenous leukemia (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

Non-limiting examples of DNA-damaging therapy and/or cytotoxic therapy may include gamma-irradiation, alkylating agents such as nitrogen mustards (chlorambucil, cyclophosphamide, ifosfamide, melphalan), nitrosoureas (streptozocin, carmustine, lomustine), alkyl sulfonates (busulfan), triazines (dacarbazine, temozolomide) and ethylenimines (thiotepa, altretamine), platinum drugs such as cisplatin, carboplatin, oxalaplatin, antimetabolites such as 5-fluorouracil, 6-mercaptopurine, capecitabine, cladribine, clofarabine, cytarabine, floxuridine, fludarabine, gemcitabine, hydroxyurea, methotrexate, pemetrexed, pentostatin, thioguanine, anthracyclines such as daunorubicin, doxorubicin, epirubicin, idarubicin, anti-tumor antibiotics such as actinomycin-D, bleomycin, mitomycin-C, mitoxantrone, topoisomerase inhibitors such as topoisomerase I inhibitors (topotecan, irinotecan) and topoisomerase II inhibitors (etoposide, teniposide, mitoxantrone), mitotic inhibitors such as taxanes (paclitaxel, docetaxel), epothilones (ixabepilone), vinca alkaloids (vinblastine, vincristine, vinorelbine), estramustine, cyclin-dependent kinase inhibitors (roscovitine, palbociclib, abemaciclib, olaparib), epigenetic modifiers (curcumin, valproic acid), and HIV medications such as NRTIs (Nucleoside Reverse Transcriptase Inhibitors), NNRTIs (Non-Nucleoside Reverse Transcriptase Inhibitors), and protease inhibitors (azidothymidine, tenofovir, emtricitabine, abacavir, nevirapine, atazanavir, lopinavir). In various embodiments, administration of one or more CRYAB inhibitors is contemplated, e.g., as an adjuvant (adjunct) to a therapeutic regimen comprising administration of one or more of the above-identified DNA-damaging therapeutics.

In certain embodiments, when chemotherapy or radiotherapy is administered in a treatment cycle of at least one day on-therapy (i.e., chemotherapy or radiotherapy)) followed by at least 3, 4, 5, 6, 7, 8, 9, 10, 11, 12, 13, 14 (or about 2 weeks), 15, 16, 17, 18, 19, 20, 21 (or about 3 weeks) days, or about 4 weeks (about one month) off-therapy (i.e., off chemo- or radio-therapy), the CRYAB inhibitor (senolytic agent) can be administered on one or more days during the off-therapy time interval (time period) beginning on or after the second day of the off-therapy time interval and ending on or before the last day of the off-therapy time interval. By way of illustrative example, if n is the number of days off-therapy, then the CRYAB inhibitor (senolytic agent) is administered on at least one day and no more than one day prior of the off-therapy time interval. In a certain particular embodiment, when chemotherapy or radiotherapy is administered in a treatment cycle of at least one day on-therapy (i.e., chemotherapy or radiotherapy) followed by at least one week off-therapy, the CRYAB inhibitor (senolytic agent) is administered on one or more days during the off-therapy time interval beginning on or after the second day of the off-therapy time interval and ending on or before the last day of the off-therapy time interval. In a more specific embodiment, when chemotherapy or radiotherapy is administered in a treatment cycle of at least one day on-therapy (i.e., chemotherapy or radiotherapy) followed by at least one week off-therapy, the CRYAB inhibitor (senolytic agent) is administered on one day that is the sixth day of the off-therapy time interval. In other specific embodiments, when chemotherapy or radiotherapy is administered in a treatment cycle of at least one day on-therapy (i.e., chemotherapy or radiotherapy)) followed by at least two weeks off-therapy, the CRYAB inhibitor (senolytic agent) is administered beginning on the sixth day of the off-chemo- or radio-therapy time interval and ending at least one day or at least two days prior to the first day of a subsequent chemotherapy or radiation therapy treatment course. By way of example, if the off-chemo- or radio-therapy time interval is two weeks, a CRYAB inhibitor (senolytic agent) may be administered on at least one and on no more than 7 days (i.e., 1, 2, 3, 4, 5, 6, or 7 days) of the off-therapy time interval beginning on the sixth day after the chemotherapy or radiotherapy course ends (i.e., the sixth day of the off chemo-radio-therapy interval). When the off-chemo- or radio-therapy time interval is at least three weeks, a CRYAB inhibitor (senolytic agent) may be administered on at least one day and on no more than 14 days (i.e., 1-14 days: 1, 2, 3, 4, 5, 6, 7, 8, 9, 10, 11, 12, 13, or 14 days) of the off-therapy time interval beginning on the sixth day after the chemotherapy or radiotherapy course ends. In other embodiments, depending on the off-chemo-radio-therapy interval, the CRYAB inhibitor (senolytic agent) treatment course is at least one day and no longer than 2, 3, 4, 5, 6, 7, 8, 9, 10, 11, 12, 13, 14, 15, 16, 17, 18, 19, 20, or no more than 21 days (i.e., 1-21 days), provided that administration of the CRYAB inhibitor (senolytic agent) is not concurrent with the chemotherapy or radiotherapy. In certain embodiments, the CRYAB inhibitor (senolytic agent)—treatment course is a single day. In certain embodiments, the CRYAB inhibitor (senolytic agent)—is administered on two or more days within a treatment window of no longer than 14 days, on 3 or more days within a treatment window of no longer than 14 days; on 4 or more days within a treatment window of no longer than 14 days; on 5 or more days within a treatment window of no longer than 14 days; on 6, 7, 8, 9, 10, 11, 12, 13, or 14 days within treatment window of no longer than 14 days. In certain embodiments, when the at least one CRYAB inhibitor (senolytic agent) is administered to a subject during a treatment course of 3 days or more, the agent may be administered every 2.^{rd} day (i.e., every other day). In other certain embodiments, when the at least one CRYAB inhibitor (senolytic agent) is administered to a subject during a treatment course of 4 days or more, the agent may be administered every 3^{rd} day (i.e., every other third day).

Many chemotherapy and radiotherapy treatment regimens comprise a finite number of cycles of on-drug therapy followed by off-drug therapy or comprise a finite timeframe in which the chemotherapy or radiotherapy is administered. Such cancer treatment regimens may also be called treatment protocols. The protocols are determined by clinical trials, drug labels, and clinical staff in conjunction with the subject to be treated. The number of cycles of a chemotherapy or radiotherapy or the total length of time of a chemotherapy or radiotherapy regimen can vary depending on the patient's response to the cancer therapy. The timeframe for such treatment regimens is readily determined by a person skilled in the oncology art. In another embodiment, for treating metastasis, a CRYAB inhibitor (senolytic agent) may be administered after the treatment regimen of chemotherapy or radiotherapy has been completed. In a particular embodiment, the senolytic agent is administered after the chemotherapy or radiotherapy has been completed on one or more days within treatment window (i.e., senolytic agent treatment course) of no longer than 14 days. In other embodiments, the senolytic agent treatment course is no longer than 2, 3, 4, 5, 6, 7, 8, 9, 10, 11, 12, 13, 14, 15, 16, 17, 18, 19, 20, or no more than 21 days. In other embodiments, the treatment course is a single day. In certain embodiments, the senolytic agent is administered on two or more days within a treatment window of no longer than 14 days, on 3 or more days within a treatment window of no longer than 14 days; on 4 or more days within a treatment window of no longer than 14 days; on 5 or more days within a treatment window of no longer than 14 days; on 6, 7, 8, 9, 10, 11, 12, 13, or 14 days within treatment window of no longer than 14 days. In certain embodiments, when the at least one senolytic agent is administered to a subject after chemotherapy or radiotherapy for a treatment window of 3 days or more, the agent may be administered every 2^{nd} day (i.e., every other day). In other certain embodiments, when the at least one senolytic agent (e.g., a CRYAB inhibitor) is administered to a subject for a treatment window of 4 days or more, the agent may be administered every 3^{rd} day (i.e., every other third day). In one embodiment, the treatment with the senolytic agent may be initiated at least 1, 2, 3, 4, 5, 6, 7, 8, 9, 10, 11, 12, 13, or 14 days or later after the cancer treatment regimen has been completed. In a more particular embodiment, the treatment with the senolytic agent may be initiated at least 6, 7, 8, 9, 10, 11, 12, 13, or 14 days or later after the cancer treatment regimen has been completed.

### Prevention or Treatment of Vascular Stiffness.

It was surprisingly discovered that senolytics, and in particular, the CRYAB inhibitors described herein (e.g., 25 HC) can reduce vascular stiffness. Accordingly, in various embodiments the use of senolytics including, but not limited to the senolytic agents described herein, can be used to reduce vascular stiffness and/or ro prevent the development of increased vascular stiffness.

Vascular stiffness, and in particular arterial stiffness, is recognized increasingly as an important component in cardiovascular risk, particularly in chronic kidney disease and end stage renal disease (ESRD) populations. Methods of measuring vascular stiffness are known to those of skill in the art, and such measurements are increasingly being used as prophylactic metrics.

When the heart contracts it generates a pulse or energy wave that travels through the circulatory system. The speed of travel of this pulse wave (pulse wave velocity (PWV)) is related to the stiffness of the arteries. Other terms that are used to describe the mechanical properties of arteries include elastance, or the reciprocal (inverse) of elastance, compliance.

Measurement of aortic PWV provides some of the strongest evidence concerning the prognostic significance of large artery stiffening. Increased aortic PWV has been shown to predict cardiovascular, and in some cases all cause, mortality in individuals with end stage kidney disease (Blacher et al. (1999) Circulation, 99(18): 2434-2439), hypertension, (Laurent et al. (2001) Hypertension, 37(5): 1236-1241), diabetes mellitus (Cruickshank et al. (2002) Circulation, 106(16): 2085-2090), and in the general population (Mattace-Raso et al. (2006) Circulation, 113(5): 657-663; Willum-Hansen et al. (2006) Circulation, 113(5): 664-670).

The primary sites of end-target organ damage following an increase in arterial stiffness are the heart, the brain (stroke, white matter hyperintensities (WMHs)), and the kidneys (age-related loss of kidney function). The mechanisms linking arterial stiffness to end-organ damage are several-fold.

Firstly, stiffened arteries compromise the Windkessel effect of the arteries (see, e.g., Belz (1995) Cardiovasc. Drug Ther. 9(1): 73-83). The Windkessel effect buffers the pulsatile ejection of blood from the heart converting it into a steadier, even outflow. This function depends on the elasticity of the arteries and stiffened arteries require a greater amount of force to permit them to accommodate the volume of blood ejected from the heart (stroke volume). This increased force requirement equates to an increase in pulse pressure (Id.). The increase in pulse pressure may result in increased damage to blood vessels in target organs such as the brain or kidneys (Mitchell (2015) Curr. Opin. Nephrol. Hyperten. 24(1): 1-7; Fernandez-Fresnedo et al. (2006) J. Am. Soc. Nephrol. 17(12 suppl3): S246-S249). This effect may be exaggerated if the increase in arterial stiffness results in reduced wave reflection and more propagation of the pulsatile pressure into the microcirculation (see, e.g., Mitchell (2015) supra.).

An increase in arterial stiffness also increases the load on the heart, since it has to perform more work to maintain the stroke volume. Over time, this increased workload causes left ventricular hypertrophy and left ventricular remodeling, which can lead to heart failure (see, e.g., Cheng & Vasan (2011) Circulation. 124(20): e516-e519). The increased workload may also be associated with a higher heart rate, a proportionately longer duration of systole and a comparative reduction of duration of diastole (see, e.g., Whelton et al. (2013) Hypertension, 62(3): 477-484). This decreases the amount of time available for perfusion of cardiac tissue, which largely occurs in diastole (see, e.g., Westerhof et al. (2010). Snapshots of Hemodynamics: An Aid for Clinical Research and Graduate Education. Springer Science & Business Media. pp. 181). Thus, the hypertrophic heart, which has a greater oxygen demand, may have a compromised supply of oxygen and nutrients.

Arterial stiffness may also affect the time at which pulse wave reflections return to the heart. As the pulse wave travels through the circulation it undergoes reflection at sites where the transmission properties of the arterial tree change (i.e., sites of impedance mismatch). These reflected waves propagate backwards towards the heart. The speed of propagation (i.e. PWV) is increased in stiffer arteries and consequently reflected waves will arrive at the heart earlier in systole. This increases the load on the heart in systole (see, e.g., Nabeel et al. (2020) IEEE Rev. Biomed. Engineering, 13: 74-112).

Thus, in certain embodiments, in addition to reducing vascular stiffness, it is believed therapeutic administration of the inhibitors of CRYAB expression and/or protein aggregation (or other senolytic agents described herein) can improve Windkessel effect, and/or reduce load on heart, and/or lower resting heart rate, and/or lower duration of systole with an accompanying increase in duration of diastole. Similarly, it is believed prophylactic administration of administration of the inhibitors of CRYAB expression and/or protein aggregation (or other senolytic agents described herein) can slow or stop a decrease in Windkessel effect, and/or slow or stop increased load on heart, and/or slow or stop increasing resting heart rate, and/or slow or stop decreases in the duration of systole with an accompanying increase in duration of diastole.

Accordingly, in various embodiments, the inhibitors of CRYAB expression and/or protein aggregation are administered to a subject (e.g., a mammal) in need thereof to therapeutically improve (reduce) vascular stiffness, or to prophylactically slow or stop increases in vascular stiffness.

While the inhibitors of CRYAB expression and/or aggregation described herein are believed to be effective for these therapeutic and/or prophylactic methods, it is also believed that other senolytic agents can be similarly effective to reduce and/or to prevent or slow the increase in vascular stiffness.

Senolytic Agents for Reducing Vascular Stiffness and/or for Slowing or Preventing Increases in Vascular Stiffness.

In certain illustrative embodiments, methods are provided that involve the administration of one or more senolytic agent(s) prophylactically to prevent an increase in vascular stiffness or therapeutically to reduce vascular stiffness.

A senolytic agent as used herein is an agent that “selectively” (preferentially or to a greater degree) destroys, kills, removes, or facilitates selective destruction of senescent cells. In other words, in certain embodiments, the senolytic agent destroys or kills a senescent cell in a biologically, clinically, and/or statistically significant manner compared with its capability to destroy or kill a non-senescent cell. In typical embodiments, a senolytic agent is used in an amount and for a time sufficient to selectively kill established senescent cells but while substantially avoiding the killing or destruction of non-senescent cell(s) in a clinically significant or biologically significant manner. In certain embodiments, the senolytic agents alter at least one signaling pathway in a manner that induces (initiates, stimulates, triggers, activates, and/or promotes) and results in death of the senescent cell(s). The senolytic agent may alter, for example, either or both of a cell survival signaling pathway (e.g., an Akt pathway) or an inflammatory pathway, for example, by antagonizing a protein within the cell survival and/or inflammatory pathway in a senescent cell.

Without wishing to be bound by a particular theory, one mechanism by which the senolytic agent(s) described herein selectively kill senescent cells is by inducing (e.g., activating, stimulating, removing inhibition of) one or more components of an apoptotic pathway that leads to cell death. In certain embodiments non-senescent cells may be proliferating cells or may be quiescent cells. In certain instances, exposure of non-senescent cells to the senolytic agent as used in the methods described herein may temporarily reduce the capability of non-senescent cell to proliferate, however, an apoptotic pathway is typically not induced and the non-senescent cell is typically not destroyed.

Certain senolytic agents that may be used in the methods described herein (e.g., for reducing vascular stiffness and/or for slowing or stopping increases in vascular stiffness) have been described as useful for treating a cancer. However, in the methods described herein, the senolytic agents are typically administered in a manner that would be considered different and likely ineffective for treating a cancer. The administration of one or more senolytic agents in the methods described herein may comprise one or more of a daily dose, or cumulative dose over a single treatment cycle, or cumulative dose of the agent from multiple treatment cycles that is less than the dose of an agent required for cancer therapy. Therefore, the likelihood is decreased that one or more adverse effects (e.g., side effects) will occur that are associated with treating a subject according to a regimen optimized for treating a cancer. In various embodiments the senolytic agents administered in the methods described herein may be administered at a lower dose than presently described in the art for cancer treatment, and/or in a manner that selectively kill senescent cells (e.g., intermittent dosing). In certain embodiments, the senolytic agents described herein may be administered at a lower cumulative dose per treatment course or treatment cycle and would likely be insufficiently cytotoxic to cancer cells to effectively treat the cancer. In other words, according to the methods described herein, the senolytic agent is not used in a manner that would be understood by a person skilled in the art as a primary therapy for treating a cancer, whether the agent is administered alone or together with one or more additional chemotherapeutic agents or radiotherapy to treat the cancer.

However, in certain embodiments, even though an agent as used in the methods described herein is not used in a manner that is sufficient to be considered as a primary cancer therapy, the methods and senolytic combinations described herein may be used in a manner (e.g., a short term course of therapy) that is useful for inhibiting metastases. A “primary therapy for cancer” as used herein means that when an agent, which may be used alone or together with one or more agents, is intended to be or is known to be an efficacious treatment for the cancer as determined by a person skilled in the medical and oncology arts, administration protocols for treatment of the cancer using the agent have been designed to achieve the relevant cancer-related endpoints. In certain embodiments, to further reduce toxicity, a senolytic agent may be administered at a site proximal to or in contact with senescent cells (not tumor cells).

In certain embodiments the senolytic agents described herein alter (e.g., interfere with/inhibit) one or more cellular pathways that are activated during the senescence process of a cell. In certain embodiments senolytic agents may alter either cell survival signaling pathway (e.g., Akt pathway) and/or an inflammatory pathway in a senescent cell. Activation of certain cellular pathways during senescence decreases or inhibits the cell's capability to induce, and ultimately undergo apoptosis. Without wishing to be bound by theory, the mechanism by which a senolytic agent selectively kills senescent cells is by inducing (e.g., activating, stimulating, removing inhibition of) an apoptotic pathway that leads to cell death. In various embodiments a senolytic agent may alter one or more signaling pathways in a senescent cell by interacting with one, two, or more target proteins in the one or more pathways, which results in removing or reducing suppression of a cell death pathway, such as an apoptotic pathway. Contacting or exposing a senescent cell to a senolytic agent to alter one, two, or more cellular pathways in the senescent cell, may restore the cell's mechanisms and pathways for initiating apoptosis. In certain embodiments, a senolytic agent is an agent that alters a signaling pathway in a senescent cell, which in turn inhibits secretion and/or expression of one or more gene products important for survival of a senescent cell. The senolytic agent may inhibit a biological activity of the gene product(s) important for survival of the senescent cell. Alternatively, the decrease or reduction of the level of the gene product(s) in the senescent cell may alter the biological activity of another cellular component, which triggers, initiates, activates, or stimulates an apoptotic pathway or removes or reduces suppression of the apoptotic pathway. In certain embodiments senolytic agents contemplated herein comprise biologically active agents that are capable of selectively killing senescent cells in the absence of linkage or conjugation to a cytotoxic moiety (e.g., a toxin or cytotoxic peptide or cytotoxic nucleic acid). In certain embodiments the senolytic agents are also active in selectively killing senescent cells in the absence of linkage or conjugation to a targeting moiety (e.g., an antibody or antigen-binding fragment thereof; cell binding peptide) that selectively binds senescent cells.

In certain embodiments, a senolytic agent used in the methods described herein is a small organic molecule. In certain embodiments, the senolytic agents include those that are activated or that are pro-drugs that are converted to the active form, e.g., by enzymes within the cell. In certain embodiments senolytic prodrug is designed so that the prodrug is converted to an active form by enzymes that are expressed at a higher level in senescent cells than in non-senescent cells.

In various embodiments senolytic agents described herein that may alter at least one signaling pathway include, but are not limited to, an agent that inhibits an activity of at least one of the target proteins within the pathway. In certain embodiments the senolytic agent may be a specific inhibitor of one or more BCL-2 anti-apoptotic protein family members wherein the inhibitor inhibits at least BCL-xL (e.g., a Bcl-2/Bcl-xL/Bcl-w inhibitor; a selective Bcl-xL inhibitor; a Bcl-xL/Bcl-w inhibitor), an Akt kinase specific inhibitor, and/or an MDM2 inhibitor. In certain embodiments, molecules such as quercetin (and analogs thereof), enzastaurin, and dasatinib are excluded and are not compounds used in the methods and compositions described herein. In other illustrative embodiments, the methods described herein involve the use of at least two senolytic agents wherein at least one senolytic agent and a second senolytic agent are each different and independently alter either one or both of a survival signaling pathway and an inflammatory pathway in a senescent cell.

### Small Senolytic Molecules

Senolytic agents that may be used in the methods described herein include, but are not limited to, small organic molecules. In various embodiments the small organic molecules (also called small molecules or small molecule compounds herein) typically have molecular weights less than about 10⁵ daltons, or less than about 10⁴ daltons, or less than about 10³ daltons. In certain embodiments, a small molecule senolytic agent does not violate the following criteria more than once: (1) no more than 5 hydrogen bond donors (the total number of nitrogen-hydrogen and oxygen-hydrogen bonds); (2) not more than 10 hydrogen bond acceptors (all nitrogen or oxygen atoms); (3) a molecular mass less than 500 daltons; (4) an octanol-water partition coefficient[5] log P not greater than 5.

### MDM2 Inhibitors

In certain embodiments, the senolytic agent(s) used in the methods described herein may comprise an MDM2 inhibitor. In certain embodiments an MDM2 (murine double minute 2) inhibitor that may be used in the methods described herein may be a small organic molecule compound that belongs to any one of the following classes of compounds, for example, an imidazoline compound (e.g., cis-imidazoline compound), a spiro-oxindole compound, a benzodiazepine compound, a piperidinone compound, a tryptamine compound, CGM097 ((S)-1-(4-chlorophenyl)-7-isopropoxy-6-methoxy-2-(4-(methyl(((1r,4S)-4-(4-methyl-3-oxopiperazin-1-yl)cyclohexyl)methyl)amino)phenyl)-1,2-dihydroisoquinolin-3(4H)-one), and related analogs. In certain embodiments, the MDM2 inhibitor is also capable of binding to and inhibiting an activity of MDMX (murine double minute X, which is also known as HDMX in humans). The human homolog of MDM2 is called HDM2 (human double minute 2) in the art. Therefore, when the subject to which the senolytic inhibitors are administered in accordance with the methods described herein is a human subject, the compounds described herein as MDM2 inhibitors may also inhibit binding of HDM2 to one or more of its ligands.

MDM2 is described in the art as an E3 ubiquitin ligase that can promote tumor formation by targeting tumor suppressor proteins, such as p53, for proteasomal degradation through the 26S proteasome (see, e.g., Haupt et al. (1997) Nature 387: 296-299; Honda et al. (1997) FEBS Lett. 420: 25-27; Kubbutat et al. (1997) Nature, 387: 299-303; and the like). MDM2 also affects p53 by directly binding to the N-terminal end of p53, which inhibits the transcriptional activation function of p53 (see, e.g., Momand et al. (1992) Cell 69: 1237-1245; Oliner et al. (1993) Nature 362: 857-860). Mdm2 is in turn regulated by p53—(see, e.g., Lahav (2008) Exp. Med. Biol. 641: 28-38). MDM2 activities, include, inter alia, activity as a ubiquitin ligase E3 toward itself and ARRB1, facilitation of nuclear export of p53; promotion of proteasome-dependent ubiquitin-independent degradation of retinoblastoma RB1 protein, inhibition of DAXX-mediated apoptosis by inducing its ubiquitination and degradation, action as a component of TRIM28/KAP1-MDM2-p53 complex involved in stabilizing p53; component of TRIM28/KAP1-ERBB4-MDM2 complex that links growth factor and DNA damage response pathways; mediation of ubiquitination and subsequent proteasome degradation of DYRK2 in the nucleus; ubiquitination of IGF1R and SNAI1 and promotion of these moieties to proteasomal degradation. MDM2 has also been reported to induce mono-ubiquitination of the transcription factor FOXO4 (see, e.g., Brenkman et al. (2008) PLOS One, 3(7): e2819). In various embodiments the MDM2 inhibitors described herein may disrupt the interaction between MDM2 and any one or more of the aforementioned cellular components.

In one embodiment, a senolytic agent useful in the methods described herein comprises an imidazoline (e.g., a cis-imidazoline). Cis-imidazoline compounds include, but are not limited to, those called nutlins in the art. Similar to other MDM2 inhibitors described herein, nutlins are cis-imidazoline small molecule inhibitors of the interaction between MDM2 and p53 (see, e.g., Vassilev et al. (2004) Science 303(5659): 844-48). Illustrative, but non-limiting examples of cis-imidazolines compounds that may be used in the methods described herein are described in U.S. Pat. Nos. 6,734,302; 6,617,346; and 7,705,007 and in U.S. Patent Pub. Nos: 2005/0282803, 2007/0129416, and 2013/0225603. In certain embodiments, the methods described herein comprise use of a nutlin compound called Nutlin-1, or a nutlin compound called Nutlin-2, or a Nutlin compound called Nutlin-3 (see, e.g., CAS Registry Nos. 675576-98-4 and 548472-68-0). The active enantiomer of Nutlin-3 (4-[[4S,5R)-4,5-bis(4-chlorophenyl)-4,5-dihydro-2-[4-methoxy-2-(-1-methylethoxy)phenyl]-1H-imidazol-1-yl]carbonyl]-2-piperazinone) is called Nutlin-3a in the art. In certain embodiments, the methods described herein comprise use of Nutlin-3a for selectively killing senescent cells.

Nutlin-3 is described in the art as a nongenotoxic activator of the p53 pathway, and the activation of p53 is controlled by the murine double minute 2 (MDM2) gene. The MDM2 protein is an E3 ubiquitin ligase and controls p53 half-life by way of ubiquitin-dependent degradation. Nutlin-3a has been investigated in pre-clinical studies (e.g., with respect to pediatric cancers) and clinical trials for treatment of certain cancers (e.g., retinoblastoma). To date in vitro and pre-clinical studies with Nutlin-3 have suggested that the compound has variable biological effects on the function of cells exposed to the compound. For example, Nutlin-3 reportedly increases the degree of apoptosis of cancer cells in hematological malignancies including B-cell malignancies (see, e.g., Zauli et al., (2011) Clin. Cancer Res. 17: 762-770 and references cited therein) and in combination with other chemotherapeutic drugs, such as dasatinib, the cytotoxic effect appears synergistic (see, e.g., Zauli et al., supra).

In certain embodiments the imidazoline compound comprises a compound having the structure:

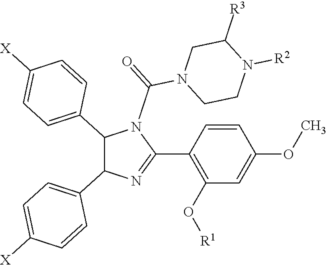

or a pharmaceutically acceptable salt thereof; where X is halide (e.g., Cl, Fl, etc.); R¹ is alkyl (e.g., C₁-C₁₂ alkyl), R² is —H or heteroalkyl, and R³ is —H or ═O. In certain embodiments, as noted above, the imidazoline compound is selected from the group consisting of nutlin-1, nutlin-2, and nutlin-3. In certain embodiments the imidazoline compound comprises a 4-[[(4S,5R)-4,5-bis(4-chlorophenyl)-4,5-dihydro-2-[4-methoxy-2-(1-methyle-thoxy)phenyl]-1H-imidazol-1-yl]carbonyl]-2-piperazinone or a pharmaceutically acceptable salt thereof.

In certain embodiments the imidazoline compound comprises a compound having the structure:

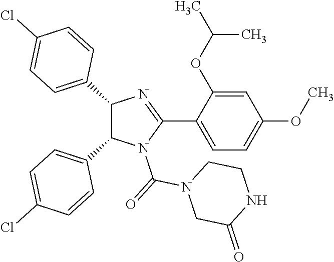

or a pharmaceutically acceptable salt thereof.

Another illustrative senolytic cis-imidazoline compound useful for the methods described herein RG-7112 (Roche) (CAS No: 939981-39-2; IUPAC name: ((4S,5R)-2-(4-(tert-butyl)-2-ethoxyphenyl)-4,5-bis(4-chlorophenyl)-4,5-di-methyl-4,5-dihydro-1H-imidazol-1-yl)(4-(3-(methylsulfonyl)propyl)piperazin--1-yl)methanone (see, e.g., U.S. Pat. No. 7,851,626; Tovar et al. (2013) Cancer Res. 72: 2587-2597).

In another illustrative embodiment, an MDM2 inhibitor useful in the methods described herein is a cis-imidazoline compound called RG7338 (Roche) (IPUAC Name: 4-((2R,3S,4R,5S)-3-(3-chloro-2-fluorophenyl)-4-(4-chloro-2-fluorophenyl)--4-cyano-5-neopentylpyrrolidine-2-carboxamido)-3-methoxybenzoic acid) (CAS 1229705-06-9); Ding et al. (2013) J. Med. Chem. 56(14): 5979-5983; Zhao et al. (2013) J. Med. Chem. 56(13): 5553-5561). Yet another illustrative nutlin compound is RO5503781 (also known as idasanutlin). Other potent cis-imidazoline small molecule compounds include dihydroimidazothiazole compounds (e.g., DS-3032b; Daiichi Sankyo) (see, also, Miyazaki et al. (2013) Bioorg. Med. Chem. Lett. 23(3): 728-732; Miyazaki et al. (2012) Bioorg. Med. Chem. Lett. 22(20): 6338-6342; PCT Pub. No: WO 2009/151069).

In certain embodiments, a cis-imidazoline compound that may be used in the methods described herein is a dihydroimidazothiazole compound.

In certain embodiments, the MDM2 small molecule inhibitor comprises a spiro-oxindole compound (see, e.g., compounds described in Ding et al. (2005) J. Am. Chem. Soc. 127: 10130-10131; Shangary et al. (2008) Proc. Natl. Acad. Sci. USA, 105: 3933-3938; Shangary et al. (2008) Mol. Cancer Ther. 7: 1533-1542; Hardcastle et al., (2005) Bioorg. Med. Chem. Lett. 15: 1515-1520; Hardcastle et al. (2006) J. Med. Chem. 49(21): 6209-6221; Watson et al. (2011) Bioorg. Med. Chem. Lett. 21(19): 5916-5919, which are incorporated herein by reference for the spiro-oxindole compounds described therein). Other examples of spiro-oxindole compounds that are MDM2 inhibitors are called in the art MI-63, MI-126; MI-122, MI-142, MI-147, MI-18, MI-219, MI-220, MI-221, and MI-773. Another specific spiro-oxindole compound is 3-(4-chlorophenyl)-3-((1-(hydroxymethyl)cyclopropyl)methoxy)-2-(4-nitrobe-nzyl)isoindolin-1-one. Another compound is called MI888 (see, e.g., Zhao et al. (2013) J. Med. Chem. 56(13): 5553-5561; PCT Pub. No: WO 2012/065022).

In certain embodiments the MDM2 small molecule inhibitor that may be used in the methods described herein comprises a benzodiazepinedione (see, e.g., Grasberger et al. (2005) J. Med. Chem. 48: 909-912; Parks et al. (2005) Bioorg. Med. Chem. Lett. 15: 765-770; Raboisson et al. (2005) Bioorg. Med. Chem. Lett. 15: 1857-1861; Koblish et al. (2006) Mol. Cancer Ther. 5: 160-169). Benzodiazepinedione compounds that may be used in the methods described herein include, but are not limited to, 1,4-benzodiazepin-2,5-dione compounds. Examples of benzodiazepinedione compounds include 5-[(3S)-3-(4-chlorophenyl)-4-[(R)-1-(4-chlorophenyl)ethyl]-2,5-dioxo-7-ph-enyl-1,4-diazepin-1-yl]valeric acid and 5-[(3S)-7-(2-bromophenyl)-3-(4-chlorophenyl)-4-[(R)-1-(4-chlorophenyl)eth-yl]-2,5-dioxo-1,4-diazepin-1-yl]valeric acid (see, e.g., Raboisson et al., supra). Other benzodiazepinedione compounds are called in the art TDP521252 (IUPAC Name: 5-[(3S)-3-(4-chlorophenyl)-4-[(1R)-1-(4-chlorophenyl)ethyl]-7-ethynyl-2,5--dioxo-3H-1,4-benzodiazepin-1-yl]pentanoic acid) and TDP665759 (IUPAC Name: (3S)-4-[(1R)-1-(2-amino-4-chlorophenyl)ethyl]-3-(4-chlorophenyl)-7--iodo-1-[3-(4-methylpiperazin-1-yl)propyl]-3H-1,4-benzodiazepine-2,5-dione) (see, e.g., Parks et al., supra; Koblish et al., supra).

In certain embodiments, the MDM2 small molecule inhibitor comprises a terphenyl (see, e.g., Yin et al. (2005) Angew Chem. Int. Ed. Engl. 44: 2704-2707; Chen et al. (2005) Mol. Cancer Ther. 4: 1019-1025). In certain embodiments, the MDM2 inhibitor that may be used in the methods described herein comprises a quilinol (see, e.g., Lu et al. (2006) J. Med. Chem. 49: 3759-3762). In certain embodiments, the MDM2 inhibitor comprises a chalcone (see, e.g., Stoll et al. (2001) Biochm. 40: 336-344). In certain embodiments, the MDM2 inhibitor is a sulfonamide (e.g., NSC279287) (see, e.g., Galatin et al. (2004) J. Med. Chem. 47: 4163-4165).

In certain embodiments, a compound that may be used in the methods described herein comprises a tryptamine, such as serdemetan (JNJ-26854165; chemical name: N1-(2-(1H-indol-3-yl)ethyl)-N4-(pyridine-4-yl)benzene-1,4-diamine; CAS No. 881202-45-5) (Johnson & Johnson, New Brunswick, N.J.). Serdemetan is a tryptamine derivative that activates p53 and acts as a HDM2 ubiquitin ligase antagonist (see, e.g., Chargari et al. (2011) Cancer Lett. 312(2): 209-218; Kojima et al. (2010) Mol. Cancer Ther. 9: 2545-2557; Yuan et al. (2011) J. Hematol. Oncol. 4:16).

In certain embodiments, MDM2 small molecule inhibitors that may be used in the methods described herein include, but are not limited to those described in Rew et al. (2012) J. Med. Chem. 55: 4936-4954; Gonzalez-Lopez de Turiso et al. (2013) J. Med. Chem. 56: 4053-4070; Sun et al. (2014) J. Med. Chem. 57: 1454-1472).

In certain embodiments, the MDM2 inhibitor comprises a piperidinone compound. An example of a potent MDM2 piperidinone inhibitor is AM-8553 ({(3R,5R,6S)-5-(3-Chlorophenyl)-6-(4-chlorophenyl)-1-[(2S,3S)-2-hydroxy-3--pentanyl]-3-methyl-2-oxo-3-piperidinyl]acetic acid; CAS No. 1352064-70-0) (Amgen, Thousand Oaks, Calif.).

In certain embodiments, an MDM2 inhibitor that may be used in the methods described herein comprises a piperidine (see, e.g., PCT Publ. No:WO 2011/046771). In certain embodiments, an MDM2 inhibitor that may be used in the methods described herein comprises an imidazole-indole compound (see, e.g., PCT Pub. No: WO 2008/119741).

Examples of compounds that bind to MDM2 and to MDMX and that may be used in the methods described herein also include, but are not limited to, RO-2443 and RO-5963 ((Z)-2-(4-((6-Chloro-7-methyl-1H-indol-3-yl)methylene)-2,5-dioxoimidazoli-din-1-yl)-2-(3,4-difluorophenyl)-N-(1,3-dihydroxypropan-2-yl)acetamide) (see, e.g., Graves et al. (2012) Proc. Natl. Acad. Sci. USA, 109: 11788-11793; Zhao et al. (2013) BioDiscovery, supra).

In another illustrative, but non-limiting embodiment, an MDM2 inhibitor referred to in the art as CGM097 may be used in the methods described herein.

### Inhibitors of BCL-2 Anti-Apoptotic Family of Proteins

In certain embodiments, the senolytic agent used in the methods described herein may be an inhibitor of one or more proteins in the BCL-2 family. In certain embodiments, at least one senolytic agent is selected from an inhibitor of one or more BCL-2 anti-apoptotic protein family members wherein the inhibitor inhibits at least BCL-xL. Typically, inhibitors of BCL-2 anti-apoptotic family of proteins alter at least a cell survival pathway. Apoptosis activation may occur via an extrinsic pathway triggered by the activation of cell surface death receptors or an intrinsic pathway triggered by developmental cues and diverse intracellular stresses. This intrinsic pathway, also known as the stress pathway or mitochondrial pathway, is primarily regulated by the BCL-2 family, a class of key regulators of caspase activation consisting of anti-apoptotic (pro-survival) proteins having BH1-BH4 domains (BCL-2 (i.e., the BCL-2 protein member of the BCL-2 anti-apoptotic protein family), BCL-xL, BCL-w, A1, MCL-1, and BCL-B); pro-apoptotic proteins having BH1, BH2, and BH3 domains (BAX, BAK, and BOK); and pro-apoptotic BH3-only proteins (BIK, BAD, BID, BIM, BMF, HRK, NOXA, and PUMA) (see, e.g., Cory et al., Nature Reviews Cancer 2:647-56 (2002); Cory et al., Cancer Cell 8:5-6 (2005); Adams et al., Oncogene 26:1324-1337 (2007)). BCL-2 anti-apoptotic proteins block activation of pro-apoptotic multi-domain proteins BAX and BAK (see, e.g., Adams et al., Oncogene 26:1324-37 (2007)). While the exact mechanism of apoptosis regulation is unknown, it is hypothesized that BH3-only proteins unleashed by intracellular stress signals bind to anti-apoptotic BCL-2 like proteins via a BH3 “ligand” to a “receptor” BH3 binding groove formed by BH1-3 regions on anti-apoptotic proteins, thereby neutralizing the anti-apoptotic proteins (see, e.g., Letai et al. (2002) Cancer Cell, 2: 183-192; Adams et al., Oncogene, supra). BAX and BAK can then form oligomers in mitochondrial membranes, leading to membrane permeabilization, release of cytochrome C, caspase activation, and ultimately apoptosis (see, e.g., Adams et al., Oncogene, supra).

As used herein, in various embodiments a BCL-2 family member that is inhibited by the agents described herein is a pro-survival (anti-apoptotic) family member. The senolytic agents used in the methods described herein inhibit one or more functions of the BCL-2 anti-apoptotic protein, BCL-xL (which may also be written herein and in the art as Bcl-xL, BCL-XL, Bcl-xl, or Bcl-XL). In certain embodiments, in addition to inhibiting BCL-xL function, the inhibitor may also interact with and/or inhibit one or more functions of BCL-2 (e.g., BCL-xL/BCL-2 inhibitors). In certain embodiments, senolytic agents used in the methods described herein are classified as inhibitors of each of BCL-xL and BCL-w (i.e., BCL-xL/BCL-w inhibitors). In certain embodiments, senolytic agents used in the methods described herein that inhibit BCL-xL may also interact with and inhibit one or more functions of each of BCL-2 (i.e., the BCL-2 protein) and BCL-w (i.e., BCL-xL/BCL-2/BCL-w inhibitors), thereby causing selective killing of senescent cells. In certain embodiments, a BCL-2 anti-apoptotic protein inhibitor interferes with the interaction between the BCL-2 anti-apoptotic protein family member (which includes at least BCL-xL) and one or more ligands or receptors to which the BCL-2 anti-apoptotic protein family member would bind in the absence of the inhibitor. In certain embodiments, an inhibitor of one or more BCL-2 anti-apoptotic protein family members wherein the inhibitor inhibits at least BCL-xL specifically binds only to one or more of BCL-xL, BCL-2, BCL-w and not to other Bcl-2 anti-apoptotic Bcl-2 family members, such as Mcl-1 and BCL2A1.

In certain embodiments, the senolytic agent used in the methods described herein is a BCL-xL selective inhibitor and inhibits one or more functions of BCL-xL. Such senolytic agents that are BCL-xL selective inhibitors do not inhibit the function of one or more other BCL-2 anti-apoptotic proteins in a biologically or statistically significant manner BCL-xL may also be called BCL2L1, BCL2-like 1, BCLX, BCL2L, BCLxL, or BCL-X herein and in the art. In one embodiment, BCL-xL selective inhibitors alter (e.g., reduce, inhibit, decrease, suppress) one or more functions of BCL-xL but do not significantly inhibit one or more functions of other proteins in the BCL-2 anti-apoptotic protein family (e.g., BCL-2 or BCL-w). In certain embodiments, a BCL-xL selective inhibitor interferes with the interaction between BCL-xL and one or more ligands or receptors to which BCL-xL would bind in the absence of the inhibitor. In certain particular embodiments, a senolytic agent that inhibits one or more of the functions of BCL-xL selectively binds to human BCL-xL but not to other proteins in the BCL-2 family, which effects selective killing of senescent cells.

BCL-xL is an anti-apoptotic member of the BCL-2 protein family. BCL-xL also plays an important role in the crosstalk between autophagy and apoptosis (see, e.g., Zhou et al. (2011) FEBS J. 278: 403-413). BCL-xL also appears to play a role in bioenergetic metabolism, including mitochondrial ATP production, Ca²⁺ fluxes, and protein acetylation, as well as on several other cellular and organismal processes such as mitosis, platelet aggregation, and synaptic efficiency (see, e.g., Michels et al. (2013) Int. J. Cell Biol., Vol. 2013, Article ID 705294). In certain embodiments, the BCL-xL inhibitors described herein may disrupt the interaction between BCL-xL and any one or more of the aforementioned BH3-only proteins to promote apoptosis in cells.

In certain embodiments, a BCL-xL inhibitor used in the methods described herein is a selective inhibitor, meaning, that it preferentially binds to BCL-xL over other anti-apoptotic BCL2 family members (e.g., BCL-2, MCL-1, BCL-w, BCL-b, and BFL-1/A1). In certain embodiments, a BCL-XL selective inhibitor exhibits at least a 5-fold, 10-fold, 50-fold, 100-fold, 1000-fold, 10000-fold, 20000-fold, or 30000-fold selectivity for binding a BCL-XL protein or nucleic acid over a BCL-2 protein or nucleic acid. In certain embodiments, a BCL-xL selective inhibitor exhibits at least a 5-fold, 10-fold, 50-fold, 100-fold, 1000-fold, 10000-fold, 20000-fold, or 30000-fold selectivity for binding a BCL-xL protein or nucleic acid over a MCL-1 protein or nucleic acid. In certain embodiments, a BCL-xL selective inhibitor exhibits at least a 5-fold, 10-fold, 50-fold, 100-fold, 1000-fold, 10000-fold, 20000-fold, or 30000-fold selectivity for binding a BCL-xL protein or nucleic acid over a BCL-w protein or nucleic acid. In certain embodiments, a BCL-xL selective inhibitor exhibits at least a 5-fold, 10-fold, 50-fold, 100-fold, 1000-fold, 10000-fold, 20000-fold, or 30000-fold selectivity for binding a BCL-XL protein or nucleic acid over a BCL-B protein or nucleic acid. In certain embodiments, a BCL-XL selective inhibitor exhibits at least a 5-fold, 10-fold, 50-fold, 100-fold, 1000-fold, 10000-fold, 20000-fold, or 30000-fold selectivity for binding a BCL-xL protein or nucleic acid over an A1 protein or nucleic acid. As described herein, in certain embodiments, an inhibitor of one or more BCL-2 anti-apoptotic protein family members wherein the inhibitor inhibits at least BCL-xL (e.g., a BCL-xL selective inhibitor) has no detectable binding to MCL-1 or to BCL2A1.

Methods for measuring binding affinity of a BCL-xL inhibitor for BCL-2 family proteins are known in the art. By way of example, binding affinity of a BCL-xL inhibitor may be determined using a competition fluorescence polarization assay in which a fluorescent BAK BH3 domain peptide is incubated with BCL-xL protein (or other BCL-2 family protein) in the presence or absence of increasing concentrations of the BCL-XL inhibitor as previously described (see, e.g., U.S. Patent Pubb: 2014/0005190; Park et al. (2013) Cancer Res. 73: 5485-5496; Wang et al. (2000) Proc. Natl. Acad. Sci. USA, 97: 7124-7129; Zhang et al. (2002) Anal. Biochem. 307: 70-75; Bruncko et al. (2007) J. Med. Chem. 50: 641-662). Percent inhibition may be determined by the equation: 1-[(mP value of well-negative control)/range)]×100%. Inhibitory constant (Kᵢ) value is determined by the formula: Kᵢ=[I]₅₀/([L]₅₀/K_{d}+[P]₀/K_{d}+1) as described in Bruncko et al. (2007) J. Med. Chem. 50: 641-662 (see, also, Wang (1995) FEBS Lett. 360: 111-114).

In certain embodiments senolytic agents (e.g., BCL-xL selective inhibitors, BCL-xL/BCL-2 inhibitors, BCL-xL/BCL-2/BCL-w inhibitors, BCL-xL/BCL-w inhibitors) used in the methods described herein (e.g., for reducing vascular stiffness or for slowing or preventing increases in vascular stiffness).

In certain embodiments, the BCL-xL inhibitor comprises a small molecule compound that is a benzothiazole-hydrazone compound, an aminopyridine compound, a benzimidazole compound, a tetrahydroquinoline compound, a phenoxyl compound, and/or related analogs.

In one embodiment, a BCL-xL selective inhibitor useful for the methods described herein comprises a benzothiazole-hydrazone inhibitor. Benzothiazole-hydrazone compounds include, but are not limited to, WEHI-539 (5-[3-[4-(aminomethyl)phenoxy]propyl]-2-[(8E)-8-(1,3-benzothiazol-2-ylhyd-razinylidene)-6,7-dihydro-5H-naphthalen-2-yl]-1,3-thiazole-4-carboxylic acid), a BH3 peptide mimetic that selectively targets BCL-xL (see, e.g., Lessene et al. (2103) Nat. Chem. Biol., 9: 390-397).

In certain embodiments, the BCL-xL selective inhibitor comprises an aminopyridine compound. One illustrative, but non-limiting, aminopyridine compound that may be used as a selective BCL-xL inhibitor comprises BXI-61 (3-[(9-amino-7-ethoxyacridin-3-yl)diazenyl]pyridine-2,6-diamine) (see, e.g., Park et al. (2013) Cancer Res. 73: 5485-5496, and U.S. Patent Pub. No: 2009/0118135).

In still other embodiments, a BCL-xL selective inhibitor that may be used in the methods described herein is a benzimidazole compound. One example of a benzimidazole compound that may be used as a selective BCL-XL inhibitor is BXI-72 (2′-(4-Hydroxyphenyl)-5-(4-methyl-1-piperazinyl)-2,5′-bi(1H-benzimidazole-) trihydrochloride) (see, e.g., Park et al. (2013) supra). In certain embodiments, the methods described herein utilize BXI-72 for selectively killing senescent cells.

In certain embodiments the BCL-xL selective inhibitor used in the methods described herein comprises a tetrahydroquinoline compound (see, e.g., U.S. Patent Publ. No. 2014/0005190). Examples of tetrahydroquinoline compounds that can be used as selective BCL-xL inhibitors are shown in Table 1 of U.S. Patent Publ. No. 2014/0005190 and described therein which is incorporated herein by reference for the compounds described therein. Other inhibitors described therein may inhibit other BCL-2 family members (e.g., BCL-2) in addition to BCL-xL.

In certain embodiments, a BCL-xL selective inhibitor used in the methods described herein comprises a phenoxyl compound. One non-limiting example of a phenoxyl compound that can be used as a selective BCL-xL inhibitor is 2[[3-(2,3-dichlorophenoxy) propyl]amino]ethanol (2,3-DCPE) (see, e.g., Wu et al. (2004) Cancer Res. 64: 1110-1113).

In still another embodiment, an inhibitor of a Bcl-2 anti-apoptotic family member that inhibits at least BCL-xL that can be used in the methods described herein is described in U.S. Pat. No. 8,232,273. In one illustrative embodiment, the inhibitor comprises a BCL-xL selective inhibitor called A-1155463 (see, e.g., Tao et al. (2014) ACS Med. Chem. Lett. 5(10): 1088-1093).

In certain embodiments senolytic agent(s) useful in the methods described herein inhibits other BCL-2 anti-apoptotic family members in addition to BCL-xL. For example, in certain embodiments the methods described herein comprise use of BCL-xL/BCL-2 inhibitors, and/or BCL-xL/BCL-2/BCL-w inhibitors, and/or BCL-xL/BCL-w inhibitors and analogs thereof. In certain embodiments, the inhibitors include compounds that inhibit BCL-2 and BCL-xL, which inhibitors may also inhibit BCL-w. Examples of these inhibitors include, but are not limited to ABT-263 (4-[4-[[2-(4-chlorophenyl)-5,5-dimethylcyclohexen-1-yl]methyl]piperazin-1--yl]-N-[4-[[(2R)-4-morpholin-4-yl-1-phenylsulfanylbutan-2-yl]amino]-3-(tri-fluoromethylsulfonyl)phenyl]sulfonylbenzamide or IUPAC, (R)-4-(4-((4′-chloro-4,4-dimethyl-3,4,5,6-tetrahydro-[1,1′-biphenyl]-2-yl-)methyl)piperazin-1-yl)-N-((4-((4-morpholino-1-(phenylthio)butan-2-yl)amin-o)-3-((trifluoromethyl)sulfonyl)phenyl)sulfonyl)benzamide) (see, e.g., Park et al. (2008) J. Med. Chem. 51: 6902-6915; Tse et al. (2008) Cancer Res. 68: 3421-3428; PCT Pub. No. WO 2009/155386; U.S. Pat. Nos. 7,390,799; 7,709,467; 7,906,505; 8,624,027; and the like), and ABT-737 (4-[4-[(4′-Chloro[1,1′-biphenyl]-2-yl)methyl]-1-piperazinyl]-N-[[4-[[(1R)--3-(dimethylamino)-1-[(phenylthio)methyl]propyl]amino]-3-nitrophenyl]sulfo-nyl]benzamide, Benzamide, 4-[4-[(4′-chloro[1,1′-biphenyl]-2-yl)methyl]-1-piperazinyl]-N-[[4-[[(1R)--3-(dimethylamino)-1-[(phenylthio)methyl]propyl]amino]-3-nitrophenyl]sulfon-yl]- or 4-[4-[[2-(4-chlorophenyl)phenyl]methyl]piperazin-1-yl]-N-[4-[[(2R)--4-(dimethylamino)-1-phenylsulfanylbutan-2-yl]amino]-3-nitrophenyl]sulfony-lbenzamide) (see, e.g., Oltersdorf et al., (2005) Nature, 435: 677-681; U.S. Pat. Nos. 7,973,161, and 7,642,260). In certain embodiments, the BCL-2 anti-apoptotic protein inhibitor is a quinazoline sulfonamide compound (see, e.g., Sleebs et al. (2011) J. Med. Chem. 54:1914-1926). In certain embodiments, the BCL-2 anti-apoptotic protein inhibitor is a small molecule compound as described in Zhou et al. (2012) J. Med. Chem. 55: 4664-4682 (see, e.g., Compound 21 (R)-4-(4-chlorophenyl)-3-(3-(4-(4-(4-((4-(dimethylamino)-1-(phenylthio)bu-tan-2-yl)amino)-3-nitrophenylsulfonamido)phenyl)piperazin-1-yl)phenyl)-5-e-thyl-1-methyl-1H-pyrrole-2-carboxylic acid), and Zhou et al. (2012) J. Med. Chem. 55: 6149-6161 (see, e.g., Compound 14 (R)-5-(4-Chlorophenyl)-4-(3-(4-(4-(4-((4-(dimethylamino)-1-(phenylthio)bu-tan-2-yl)amino)-3-nitrophenylsulfonamido)phenyl)piperazin-1-yl)phenyl)-1-e-thyl-2-methyl-1H-pyrrole-3-carboxylic acid; Compound 15 (R)-5-(4-Chlorophenyl)-4-(3-(4-(4-(4-(4-(dimethylamino)-1-(phenylthio)but-an-2-yl)amino)-3-nitrophenylsulfonamido)phenyl)piperazin-1-yl)phenyl)-1-is-opropyl-2-methyl-1H-pyrrole-3-carboxylic acid). In certain embodiments, the BCL-2 anti-apoptotic protein inhibitor is a BCL-2/BCL-xL inhibitor such as BM-1074 (see, e.g., Aguilar et al. (2013) J. Med. Chem. 56: 3048-3067); BM-957 (see, e.g., Chen et al. (2012) J. Med. Chem. 55: 8502-8514); BM-1197 (see, e.g., Bai et al. (2014) PLoS One, 9(6):e99404; U.S. Patent Pub. No. 2014/0199234); N-acylsufonamide compounds (see, e.g., PCT Pub. Nos. WO 2002/024636, WO 2005/049593, and WO 2005/049594, and U.S. Pat. Nos. 7,767,684, and 7,906,505). In certain embodiments, the BCL-2 anti-apoptotic protein inhibitor is a small molecule macrocyclic compound (see, e.g., PCT Pub. No. WO 2006/127364, and U.S. Pat. No. 7,777,076). In certain embodiments, the BCL-2 anti-apoptotic protein inhibitor comprises an isoxazolidine compound (see, e.g., PCT Pub. No. WO 2008/060569; U.S. Pat. Nos. 7,851,637, and 7,842,815).

In certain embodiments, the senolytic agent comprises a compound that is an inhibitor of Bcl-2, Bcl-w, and Bcl-xL, such as ABT-263 or ABT-737. In certain specific embodiments, the senolytic agent comprises a compound or a pharmaceutically acceptable salt, stereoisomer, tautomer, or prodrug thereof as illustrated below, which depicts the structure of ABT-263. ABT-263 is also known as Navitoclax in the art.

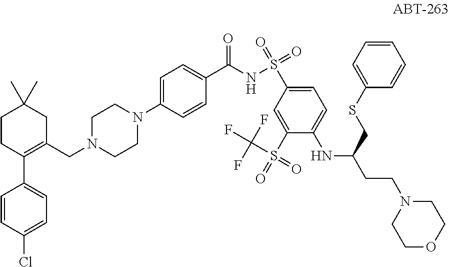 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

### Akt Kinase Inhibitors

In certain embodiments the senolytic agent comprises an Akt Kinase inhibitor. In some embodiments, the senolytic agent comprises a compound that selectively inhibits Akt1, Akt2, and/or Akt3, relative to other protein kinases.

Akt inhibitors (also known as Akt kinase inhibitors or AKT kinase inhibitors) can be divided into six major classes based on their mechanisms of action (see, e.g., Bhutani et al. (2013) Infectious Agents and Cancer, 8: 49). Akt is also called protein kinase B (PKB) in the art. The first class contains ATP competitive inhibitors of Akt and includes compounds such as CCT128930 and GDC-0068, which inhibit Akt2 and Akt1. This category also includes the pan-Akt kinase inhibitors such as GSK2110183 (afuresertib), GSK690693, and AT7867. The second class contains lipid-based Akt inhibitors that act by inhibiting the generation of PIP3 by PI3K. This mechanism is employed by phosphatidylinositol analogs such as Calbiochem Akt Inhibitors I, II and III or other PI3K inhibitors such as PX-866. This category also includes compounds such as Perifosine (KRX-0401) (Aeterna Zentaris/Keryx). The third class contains a group of compounds called pseudosubstrate inhibitors. These include compounds such as AKTide-2 T and FOXO3 hybrid. The fourth class consists of allosteric inhibitors of AKT kinase domain, and include compounds such as MK-2206 (8-[4-(1-aminocyclobutyl)phenyl]-9-phenyl-2H-[1,2,4]triazolo[3,4-f][1,6]n-aphthyridin-3-one; dihydrochloride) (Merck & Co.) (see, e.g., U.S. Pat. No. 7,576,209, which is incorporated herein by reference for the compounds described therein). The fifth class consists of antibodies and includes molecules such as GST-anti-Akt1-MTS. The last class comprises compounds that interact with the PH domain of Akt, and includes Triciribine and PX-316. Other compounds described in the art that act as AKT inhibitors include, for example, GSK-2141795 (GlaxoSmithKline), VQD-002, miltefosine, AZD5363, GDC-0068, and API-1.

In on illustrative, but non-limiting embodiment, the senolytic agent is a compound is an Akt kinase inhibitor having the structure as shown below (also called MK-2206 herein and in the art), 8-[4-(1-aminocyclobutyl)phenyl]-9-phenyl-2H-[1,2,4]triazolo[3,4-f][1,6]na-phthyridin-3-one) or a pharmaceutically acceptable salt, stereoisomer, tautomer, or prodrug thereof. The dihydrochloride salt is shown.

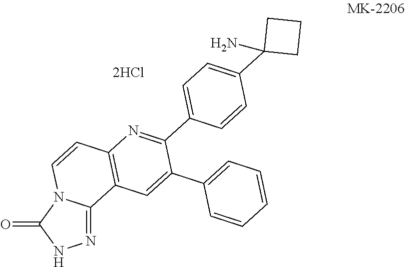 [SMILES: NC1(c2ccc(-c3nc4ccn5c(=O)[nH]nc5c4cc3-c3ccccc3)cc2)CCC1]

### Combinations of Senolytic Agents.

In certain embodiments, at least one senolytic agent may be administered with at least one other senolytic agent. In certain embodiments the two or more senolytic agents act additively or synergistically to selectively kill senescent cells. In particular embodiments, the methods described herein utilize a senolytic agent that alters either a cell survival signaling pathway or an inflammatory pathway or alters both the cell survival signaling pathway and the inflammatory pathway in a senescent cell. In certain embodiments, the methods described herein comprise use of at least two senolytic agents wherein at least one senolytic agent and a second senolytic agent are each different and independently alter either one or both of a survival signaling pathway and an inflammatory pathway in a senescent cell. For convenience, when two or more senolytic agents are described herein as being used in combination, one senolytic agent can be called a first senolytic agent, another senolytic agent can be called the second senolytic agent, etc. In other certain embodiments, the methods described herein comprise administering at least three senolytic agents (a first senolytic agent, second senolytic agent, and third senolytic agent). The adjectives, first, second, third, and such, in this context are used for convenience only and are not to be construed as describing order or administration, preference, or level of senolytic activity or other parameter unless expressly described otherwise. In particular embodiments, when two or more senolytic agents are used in the methods described herein, each senolytic agent is a small molecule.

In certain embodiments, the methods described herein comprise administering at least three senolytic agents (a first senolytic agent, second senolytic agent, and third senolytic agent). In certain embodiments, use of at least two senolytic agents results in significantly increased killing of senescent cells compared with use of each senolytic agent alone. In other particular embodiments, use of at least two senolytic agents results in significant killing of senescent cells compared with use of each senolytic agent alone and which effect may be additive or synergistic. In certain embodiments, at least two senolytic agents are each different and selected from (1) an inhibitor of one or more BCL-2 anti-apoptotic protein family members wherein the inhibitor inhibits at least BCL-xL; (for example, a Bcl-2/Bcl-xL/Bcl-w inhibitor, a Bcl-2/Bcl-xL inhibitor, a selective Bcl-xL inhibitor, or a Bcl-xL/Bcl-w inhibitor); an Akt kinase specific inhibitor; a MDM2 inhibitor. In one particular embodiment, when at least one senolytic agent administered to a subject is an inhibitor of one or more BCL-2 anti-apoptotic protein family members wherein the inhibitor inhibits at least BCL-XL (e.g., a Bcl-2/Bcl-xL/Bcl-w inhibitor, a Bcl-2/Bcl-xL inhibitor, a selective Bcl-xL inhibitor, or a Bcl-xL/Bcl-w inhibitor), a second senolytic agent is administered. In other certain embodiments, one of the two senolytic agents is an inhibitor of one or more BCL-2 anti-apoptotic protein family members wherein the inhibitor inhibits at least BCL-xL and the second senolytic agent is an MDM2 inhibitor. In yet still more particular embodiments, when at least one senolytic agent administered to a subject is a selective Bcl-xL inhibitor, a second senolytic agent is administered. In still more particular embodiments, when at least one senolytic agent administered to a subject is an MDM2 inhibitor, a second senolytic agent is administered. In still more particular embodiments, when at least one senolytic agent administered to a subject is an Akt kinase inhibitor, a second senolytic agent is administered. In even more particular embodiments, the inhibitor of one or more BCL-2 anti-apoptotic protein family members wherein the inhibitor inhibits at least BCL-xL is used alone or in combination with another senolytic agent that is also an inhibitor of one or more BCL-2 anti-apoptotic protein family members wherein the inhibitor inhibits at least BCL-xL or is a different senolytic agent as described herein. In particular embodiments, an inhibitor of one or more BCL-2 anti-apoptotic protein family members wherein the inhibitor inhibits at least BCL-xL is combined with an inhibitor of Akt kinase. By way of non-limiting example, the Bcl-2/Bcl-xL/Bcl-w inhibitor ABT-263 may be used in combination with an Akt kinase inhibitor (e.g., MK2206).

In still other particular embodiments, an MDM2 inhibitor that is a senolytic agent is used in combination with at least one additional senolytic agent in the methods described herein. The additional senolytic agent (which may be referred to for convenience as a second senolytic agent) may be another MDM2 inhibitor or may be a senolytic agent that is not a MDM2 inhibitor. In one embodiment, an inhibitor of a Bcl-2 anti-apoptotic family member that inhibits at least Bcl-xL is used in combination with an AKT inhibitor. In a more specific embodiment, the inhibitor of a Bcl-2 anti-apoptotic family member is ABT-263, ABT-737, or WEHI-539 and the AKT inhibitor is MK-2206.

In other certain embodiments, the methods described herein comprise administering at least three senolytic agents (a first senolytic agent, second senolytic agent, and third senolytic agent).

### mTOR, NFκB, and PI3-k Pathway Inhibitors

In certain embodiments a compound that may be used together with a senolytic agent described herein in the methods described herein (e.g., for selectively killing senescent cells) may comprise a compound that inhibits one or more of mTOR, NFκB, and PI3-k pathways. Thus, for example, in the methods described herein, administration of a senolytic agent (e.g., for selectively killing senescent cells) may comprise administering to the subject at issue at least one senolytic agent and an inhibitor of one or more of mTOR, NFκB, and PI3-k pathways. Inhibitors of these pathways are known in the art.

Examples of mTOR inhibitors include, but are not limited to, sirolimus, temsirolimus, everolimus, ridaforolimus, 32-deoxorapamycin, zotarolimus, PP242, INK128, PP30, Torinl, Ku-0063794, WAY-600, WYE-687 and WYE-354. Inhibitors of an NF.kappa.B pathway include, for example, NF.kappa.B activity abrogation through TPCA-1 (an IKK2 inhibitor); BAY 11-7082 (an IKK inhibitor poorly selective for IKK1 and IKK2); and MLN4924 (an NEDD8 activating enzyme (NAE)-inhibitor), and MG132.

Examples of inhibitors of PI3-k that may also inhibit mTOR and/or AKT pathways include, but are not limited to, perifosine (KRX-0401), idelalisib, PX-866, IPI-145, BAY 80-6946, BEZ235, RP6530, TGR 1201, SF1126, INK1117, GDC-0941, BKM120, XL147 (SAR245408), XL765 (SAR245409), Palomid 529, GSK1059615, GSK690693, ZSTK474, PWT33597, IC87114, TG100-115, CAL263, RP6503, PI-103, GNE-477, CUDC-907, AEZS-136, BYL719, BKM120, GDC-0980, GDC-0032, and MK2206.

### Polypeptides, Antibodies, and Nucleic Acids

In certain embodiments, a senolytic agent used in the methods described herein can comprise a polypeptide, peptide, antibody, antigen-binding fragment, peptibody, recombinant viral vector, or a nucleic acid. In certain embodiments, the senolytic agent comprises an antisense oligonucleotide, siRNA, shRNA, or a peptide. For example, senolytic agents such as polypeptides, antibodies, nucleic acids, and the like, include, for example, MDM2 inhibitors, BCL-2 family inhibitors, or Akt kinase inhibitors. In other embodiments, polypeptides, peptides, antibodies (including antigen-binding fragments thereof) that specifically bind to a ligand or target protein of a small molecule senolytic agent described herein, may be used in assays and methods for characterizing or monitoring the use of the small molecule senolytic agent.

A polynucleotide or oligonucleotide that specifically hybridizes to a portion of mRNA that encodes a target protein (e.g., Bcl-xL, Bcl-2, Bcl-w, MDM2, Akt) of a cell that is a senescent cell or that is a cell in a disease microenvironment may induce the cell to senescence by aging, a biologically damaging (i.e., cell damaging) medical therapy, or an environmental insult. In other embodiments, the target protein may be a ligand, or protein either downstream or upstream in a cell survival pathway or inflammatory pathway or apoptotic pathway. Polynucleotides and oligonucleotides may be complementary to at least a portion of a nucleotide sequence encoding a target polypeptide (e.g., a short interfering nucleic acid, an antisense polynucleotide, a ribozyme, or a peptide nucleic acid) and that may be used to alter gene and/or protein expression. These polynucleotides that specifically bind to or hybridize to nucleic acid molecules that encode a target polypeptide may be prepared using the nucleotide sequences available in the art. In another embodiment, nucleic acid molecules such as aptamers that are not sequence-specific may also be used to alter gene and/or protein expression.

Antisense polynucleotides bind in a sequence-specific manner to nucleic acids such as mRNA or DNA. Identification of oligonucleotides and ribozymes for use as antisense agents and identification of DNA encoding the target gene for targeted delivery involve methods well known in the art. For example, the desirable properties, lengths, and other characteristics of such oligonucleotides are well known. Antisense technology can be used to control gene expression through interference with binding of polymerases, transcription factors, or other regulatory molecules (see, e.g., Gee et al., In Huber and Carr, Molecular and Immunologic Approaches, Futura Publishing Co. (Mt. Kisco, NY; 1994)).

Short interfering RNAs may be used for modulating (decreasing or inhibiting) the expression of a gene encoding a target polypeptide of interest. Small nucleic acid molecules, such as short interfering RNA (siRNA), micro-RNA (miRNA), and short hairpin RNA (shRNA) molecules may be used according to the methods described herein to modulate the expression of a target protein. A siRNA polynucleotide preferably comprises a double-stranded RNA (dsRNA) but may comprise a single-stranded RNA (see, e.g., Martinez et al. (2002) Cell 110: 563-574). An siRNA polynucleotide may comprise other naturally occurring, recombinant, or synthetic single-stranded or double-stranded polymers of nucleotides (ribonucleotides or deoxyribonucleotides or a combination of both) and/or nucleotide analogues as provided herein and known and used by persons skilled in the art.

The term “siRNA” refers to a double-stranded interfering RNA unless otherwise noted. Typically, an siRNA is a double-stranded nucleic acid molecule comprising two nucleotide strands, each strand having about 19 to about 28 nucleotides (i.e., about 19, 20, 21, 22, 23, 24, 25, 26, 27, or 28 nucleotides). In certain embodiments, each strand is 19, 20, 21, 22, or 23 nucleotides. In other particular embodiments, the siRNA comprises two nucleotide strands, each strand having about 15, 16, 17, or 18 nucleotides. In other certain embodiments, one strand of the double stranded siRNA is at least two nucleotides longer, for example, one strand may have a two-base overhang (such as TT) at one end, usually the 3′ terminal end.

Short hairpin interfering RNA molecules comprise both the sense and antisense strands of an interfering RNA in a stem-loop or hairpin structure (e.g., a shRNA). An shRNA may be expressed from a DNA vector in which the DNA oligonucleotides encoding a sense interfering RNA strand are linked to the DNA oligonucleotides encoding the reverse complementary antisense interfering RNA strand by a short spacer. If needed, 3′ terminal T's and nucleotides forming restriction sites may be added. The resulting RNA transcript folds back onto itself to form a stem-loop structure.

In addition to siRNA molecules, other interfering RNA and RNA-like molecules can interact with RISC and silence gene expression, such as short hairpin RNAs (shRNAs), single-stranded siRNAs, microRNAs (miRNAs), and dicer-substrate 27-mer duplexes. Such RNA-like molecules may contain one or more chemically modified nucleotides, one or more non-nucleotides, one or more deoxyribonucleotides, and/or one or more non-phosphodiester linkages. RNA or RNA-like molecules that can interact with RISC and participate in RISC-related changes in gene expression may be referred to herein as “interfering RNAs” or “interfering RNA molecules.” Single-stranded interfering RNA in certain instances effects mRNA silencing, but less efficiently than double-stranded RNA.

A person skilled in the art will also recognize that RNA molecules, such as siRNA, miRNA, shRNA, may be chemically modified to confer increased stability against nuclease degradation while retaining the capability to bind to the target nucleic acids that may be present in cells. The RNA may be modified at any position of the molecule so long as the modified RNA binds to the target sequence of interest and resists enzymatic degradation. Modifications in the siRNA may be in the nucleotide base, the ribose, or the phosphate. By way of example, the T position of ribose can be modified, which modification can be accomplished using any one of a number of different methods routinely practiced in the art. An RNA may be chemically modified by the addition of a halide such as fluoro. Other chemical moieties that have been used to modify RNA molecules include methyl, methoxyethyl, and propyl groups (see, e.g., U.S. Pat. No. 8,675,704).

In certain embodiments, the polynucleotide or oligonucleotide (e.g., including a shRNA) may be delivered by a recombinant vector into which the polynucleotide or oligonucleotide of interest has been incorporated. In other embodiments, the recombinant viral vector may be a recombinant expression vector into which a polynucleotide sequence that encodes an antibody, an antigen-binding fragment, polypeptide or peptide that inhibits a protein in a cell survival pathway or an inflammatory pathway, including the proteins described herein such as Bcl-xL, Bcl-2, Bcl-w, MDM2, and Akt is inserted such that the encoding sequence is operatively linked with one or more regulatory control sequences to drive expression of the polypeptide, antibody, an antigen-binding fragment, or peptide. The recombinant vector or the recombinant expression vector may be a viral recombinant vector or a viral recombinant expression vector. Illustrative viral vectors include, without limitation, a lentiviral vector genome, poxvirus vector genome, vaccinia virus vector genome, adenovirus vector genome, adenovirus-associated virus vector genome, herpes virus vector genome, and alpha virus vector genome. Viral vectors may be live, attenuated, replication conditional or replication deficient, and typically is a non-pathogenic (defective), replication competent viral vector. Procedures and techniques for designing and producing such viral vectors are well known to and routinely practiced by persons skilled in the art.

In certain embodiments a senolytic agent that may be used in the methods described herein comprises an antisense oligonucleotide. By way of non-limiting example, BCL-xL specific antisense oligonucleotides that have been previously described may be used in the methods described herein (see, e.g., PCT Publ. No. WO 00/66724; Xu et al., (2001) Intl. J. Cancer 94: 268-274; Olie et al. (2002) J. Invest. Dermatol. 118: 505-512; and Wacheck et al. (2003) Br. J. Cancer, 89: 1352-1357).

In certain embodiments, a senolytic agent that may be used in the methods described herein comprises a peptide. By way of example and in certain embodiments, a BCL-xL selective peptide inhibitor is a BH3 peptide mimetic. Examples of BCL-xL selective BH3 peptide mimetics include those previously described (see, e.g., Kutzki et al., (2002) J. Am. Chem. Soc. 124: 11838-11839; Yin et al. (2004) Bioorg. Med. Chem. Lett. 22: 1375-1379; Matsumura et al. (2010) FASEB J. 7: 2201).

In certain embodiments, a senolytic agent useful in the methods described herein does not include a polynucleotide, or a fragment thereof, that encodes the exonuclease, EXO1, or a vector (including a viral vector) that comprises a polynucleotide that encodes the EXO1 enzyme (i.e., a polynucleotide encoding an EXO1 enzyme, a fragment of the polynucleotide, or a vector containing such a polynucleotide is excluded). In certain embodiments a senolytic agent useful in the methods described herein also does not include the EXO1 enzyme polypeptide (i.e., the EXO1 enzyme is excluded) or biologically active peptide or polypeptide fragment thereof. In certain embodiments such molecules are not inhibitors of one or both of a cell signaling pathway, such as an inflammatory pathway or a cell survival pathway; instead EXO1 encodes a 5′-3′ exonuclease that degrades capping defective telomeres (see, e.g., PCT Pub No: WO 2006/018632).

A senolytic useful in the methods described herein may comprise a polypeptide that is an antibody, or antigen-binding fragment. In certain embodiments the antigen-binding fragment may be an F(ab′)2, Fab, Fab′, Fv, or Fd and can also include a peptide or polypeptide that comprises at least one complementary determining region (CDR). The antibody may be an internalizing antibody or antigen-binding fragment that is internalized by the senescent cell via interaction with a target protein.

Binding properties of an antibody to its cognate antigen, may generally be determined and assessed using methods that may be readily performed by those having ordinary skill in the art (see, e.g., Harlow et al., Antibodies: A Laboratory Manual, Cold Spring Harbor Laboratory (1988)). As used herein, an antibody is said to be “immunospecific,” “specific for” or to “specifically bind” to an antigen if it reacts at a detectable level with the polypeptide Affinities of antibodies and antigen binding fragments thereof can be readily determined using conventional techniques, for example, those described by Scatchard et al. (1949) Ann. N.Y. Acad. Sci. USA 51: 660, and by surface plasmon resonance (SPR; BIAcore™, Biosensor, Piscataway, N.J.).

In various embodiments the antibodies may be polyclonal or monoclonal. A variable region or one or more complementarity determining regions (CDRs) may be identified and isolated from antigen-binding fragment or peptide libraries. An antibody, or antigen-binding fragment, may be recombinantly engineered and/or recombinantly produced. An antibody may belong to any immunoglobulin class, for example IgG, IgE, IgM, IgD, or IgA and may be obtained from or derived from an animal, for example, fowl (e.g., chicken) and mammals, which include but are not limited to a mouse, rat, hamster, rabbit, or other rodent, a cow, horse, sheep, goat, camel, human, or other primate. For use in human subjects, antibodies and antigen-binding fragments are typically human, humanized, or chimeric to reduce an immunogenic response by the subject to non-human peptides and polypeptide sequences.

In various embodiments the antibody may be a monoclonal antibody that is a human antibody, humanized antibody, chimeric antibody, bispecific antibody, or an antigen-binding fragment (e.g., F(ab′)2, Fab, Fab′, Fv, and Fd) prepared or derived therefrom. An antigen-binding fragment may also be any synthetic or genetically engineered protein (see, e.g., Hayden et al. (1997) Curr. Opin. Immunol. 9: 201-212; Coloma et al. (1997) Nat. Biotechnol. 15: 159-163; U.S. Pat. No. 5,910,573; Holliger et al., (1997) Cancer Immunol. Immunother. 45: 128-130; Drakeman et al. (1997) Exp. Opin. Investig. Drugs, 6: 1169-1178; Koelemij et al. (1999) J. Immunother. 22: 514-524; Marvin et al. (2005) Acta Pharmacol. Sin. 26:649-658; Das et al., (2005) Meth. Mol. Med. 109: 329-346; PCT Pub Nos: PCT/US91/08694 and PCT/US91/04666) and from phage- or yeast-display libraries (see, e.g., Scott et al. (1990) Science, 249: 386-390; Devlin et al., (1990) Science 249: 404-406; Cwirla et al. (1997) Science, 276: 1696-1699; U.S. Pat. Nos. 5,223,409, 5,733,731, 5,498,530, 5,432,018, 5,338,665, and 5,922,545, PCT Pub Nos: WO 96/40987 and WO 98/15833). A peptide that is a minimal recognition unit or a CDR (i.e., any one or more of the three CDRs present in a heavy chain variable region and/or one or more of the three CDRs present in a light chain variable region) may be identified by computer modeling techniques, which can be used for comparing and predicting a peptide sequence that will specifically bind to a target protein of interest (see, e.g., Bradley et al., (2005) Science 309: 1868; Schueler-Furman et al. (2005) Science 310: 638). Useful strategies for designing humanized antibodies are described in the art (see, e.g., Jones et al., (1986) Nature 321: 522-525; Riechmann et al. (1988) Nature, 332: 323-327; Padlan et al., (1995) FASEB 9: 133-139; Chothia et al. (1989) Nature, 342: 377-383).

### Senolytic Viruses.

In certain embodiments the senolytic agents include engineered senolytic viruses that specifically kill senescent cells. Such viruses are described, inter alia, in U.S. Patent Pub. No: US 2015/0064137 A1.

The foregoing senolytic agents described above are illustrative and non-limiting. Using the teachings provided herein numerous other senolytic agents useful in the methods described herein (e.g., for reducing vascular stiffness or for slowing or preventing increases in vascular stiffness) will be known to those of skill in the art.

Delaying the Onset or Progression and/or Treatment of Age-Related Diseases

It has been demonstrated that senescent cells drive age-related pathologies, and that selective elimination of these cells can prevent or delay age-related deterioration. Thus, senescent cells may be therapeutic targets in the treatment of aging and age-related disease. As such, removal of senescent cells may delay tissue dysfunction and extend health span. Clearance of senescent cells is expected to improve tissue milieu, thereby improving the function of the remaining non-senescent cells.

In various embodiments, methods are provided for delaying at least one feature of aging in a subject and/or for ameliorating one or more symptoms of aging in a subject, where the method involves administering a therapeutically effective amount of a CRYAB inhibitor to a subject. As used herein, “a feature of aging” may include, but is not limited to, systemic decline of the immune system, muscle atrophy and decreased muscle strength, decreased skin elasticity, delayed wound healing, retinal atrophy, reduced lens transparency, reduced hearing, osteoporosis, sarcopenia, hair graying, skin wrinkling, poor vision, frailty, and cognitive impairment.

An age-related pathology may include any disease or condition that is fully or partially mediated by the induction or maintenance of a non-proliferating or senescent state in a cell or a population of cells in a subject. Non-limiting examples include age-related tissue or organ decline which may lack visible indication of pathology, or overt pathology such as a degenerative disease or a function-decreasing disorder. For example, Alzheimer's disease, Parkinson's disease, cataracts, macular degeneration, glaucoma, atherosclerosis, acute coronary syndrome, myocardial infarction, stroke, hypertension, idiopathic pulmonary fibrosis (IPF), chronic obstructive pulmonary disease (COPD), osteoarthritis, sarcopenia, type 2 diabetes, obesity, fat dysfunction, coronary artery disease, cerebrovascular disease, periodontal disease, and cancer treatment-related disability such as atrophy and fibrosis in various tissues, brain and heart injury, and therapy-related myelodysplastic syndromes. Additionally, an age-related pathology may include an accelerated aging disease such as progeroid syndromes (i.e. Hutchinson-Gilford progeria syndrome, Werner syndrome, Bloom syndrome, Rothmund-Thomson Syndrome, Cockayne syndrome, xeroderma pigmentosum, trichothiodystrophy, combined xeroderma pigmentosum-Cockayne syndrome, restrictive dermopathy), ataxia telangiectasia, Fanconi anemia, Friedreich's ataxia, dyskeratosis congenital, aplastic anemia, IPF, and others. A method of identifying an age-related disease or condition as described herein may include detecting the presence of senescent cells.

Age related diseases or conditions can also include, for example, renal dysfunction, kyphosis, herniated intervertebral disc, frailty, hair loss, hearing loss, vision loss (blindness or impaired vision), muscle fatigue, skin conditions, skin nevi, diabetes, metabolic syndrome, and sarcopenia. Vision loss refers to the absence of vision when a subject previously had vision. Various scales have been developed to describe the extent of vision and vision loss based on visual acuity. Age-related diseases and conditions also include dermatological conditions, for example without limitation, treating one or more of the following conditions: wrinkles, including superficial fine wrinkles; hyperpigmentation; scars; keloid; dermatitis; psoriasis; eczema (including seborrheic eczema); rosacea; vitiligo; ichthyosis vulgaris; dermatomyositis; and actinic keratosis.

Frailty has been defined as a clinically recognizable state of increased vulnerability resulting from aging-associated decline in reserve and function across multiple physiologic systems that compromise a subject's ability to cope with every day or acute stressors. Frailty may be characterized by compromised energetics characteristics such as low grip strength, low energy, slowed waking speed, low physical activity, and/or unintentional weight loss. Studies have suggested that a patient may be diagnosed with frailty when three of five of the foregoing characteristics are observed (see, e.g., Fried et al. (2001) J. Gerontol. A Biol. Sci. Med, Sci. 56(3): M146-M156; Xue (2011) Clin. Geriatr. Med. 27(1): 1-15). In certain embodiments, aging and diseases and disorders related to aging may be treated or prevented (i.e., the likelihood of occurrence of is reduced) by administering a CRYAB inhibitor (senolytic agent). The CRYAB inhibitor agent may inhibit senescence of adult stem cells or inhibit accumulation, kill, or facilitate removal of adult stem cells that have become senescent. See, e.g., Park et al., J. Clin. Invest. 113:175-79 (2004) and Sousa-Victor, Nature 506:316-21 (2014) describing importance of preventing senescence in stem cells to maintain regenerative capacity of tissues.

The effectiveness of a senolytic agent (e.g., a CRYAB inhibitor) with respect to treating a senescence-associated disease or disorder described herein can readily be determined by a person skilled in the medical and clinical arts. One or any combination of diagnostic methods appropriate for the particular disease or disorder, which methods are well known to a person skilled in the art, including physical examination, patient self-assessment, assessment and monitoring of clinical symptoms, performance of analytical tests and methods, including clinical laboratory tests, physical tests, and exploratory surgery, for example, may be used for monitoring the health status of the subject and the effectiveness of the senolytic agent. The effects of the methods of treatment described herein can be analyzed using techniques known in the art, such as comparing symptoms of patients suffering from or at risk of a particular disease or disorder that have received the pharmaceutical composition comprising a senolytic agent with those of patients who were not treated with the senolytic agent or who received a placebo treatment.

Therapeutic benefit for subjects to whom the senolytic agents (CRYAB inhibitors) described herein are administered, includes, for example, an improved clinical outcome, wherein the object is to prevent or slow or retard (lessen) an undesired physiological change associated with the disease, or to prevent or slow or retard (lessen) the expansion or severity of such disease. As discussed herein, effectiveness of the one or more senolytic agents may include beneficial or desired clinical results that comprise, but are not limited to, abatement, lessening, or alleviation of symptoms that result from or are associated with the disease to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. The effectiveness of the senolytic agents described herein may also mean prolonging survival when compared to expected survival if a subject were not receiving the senolytic agent that selectively kills senescent cells.

In certain embodiments, administration of a senolytic agent (CRYAB inhibitor) described herein can prolong survival when compared to expected survival if a subject were not receiving treatment. Subjects in need of treatment include those who already have the disease or disorder as well as subjects prone to have or at risk of developing the disease or disorder, and those in which the disease, condition, or disorder is to be treated prophylactically. A subject may have a genetic predisposition for developing a disease or disorder that would benefit from clearance of senescent cells or may be of a certain age wherein receiving a senolytic agent would provide clinical benefit to delay development or reduce severity of a disease, including an age-related disease or disorder.

In another embodiment, a method is provided for treating a senescence-associated disease or disorder that further comprises identifying a subject who would benefit from treatment with a senolytic agent described herein (i.e., phenotyping; individualized treatment). This method comprises first detecting the level of senescent cells in the subject, such as in a particular organ or tissue of the subject. A biological sample may be obtained from the subject, for example, a blood sample, serum or plasma sample, biopsy specimen, body fluids (e.g., lung lavage, ascites, mucosal washings, synovial fluid, vitreous fluid, spinal fluid), bone marrow, lymph nodes, tissue explant, organ culture, or any other tissue or cell preparation from a subject. The level of senescent cells may be determined according to any of the in vitro assays or techniques described herein. For example, senescence cells may be detected by morphology (as viewed by microscopy, for example); production of senescence associated markers such as, senescence-associated β-galactosidase (SA-β-gal), p16INK4a, p21, PAI-1, or any one or more SASP factors (e.g., IL-6, MMP3). The senescent cells and non-senescent cells of the biological sample may also be used in an in vitro cell assay in which the cells are exposed to any one of the senolytic agents described herein to determine the capability of the senolytic agent to kill the subject's senescent cells without undesired toxicity to non-senescent cells. As positive controls in these assays, the assay may incorporate any one of the senolytic agents (e.g., Nutlin-3a, RG-7112, ABT-263, ABT-737, WEHI-539, A-1155463, MK-2206) described herein. The subject then may be treated with an appropriate senolytic agent, which may be a MDM2 inhibitor; an inhibitor of one or more Bcl-2 anti-apoptotic protein family members wherein the inhibitor inhibits at least Bcl-xL (e.g., a Bcl-xL selective inhibitor, Bcl-2/Bcl-xL/Bcl-w inhibitor, a Bcl-2/Bcl-xL or a Bcl-xL/Bcl-w inhibitor); or an Akt specific inhibitor. In addition, these methods may be used to monitor the level of senescent cells in the subject before, during, and after treatment with a senolytic agent. In certain embodiments, the presence of senescence cells, may be detected (e.g., by determining the level of a senescent cell marker expression of mRNA, for example), and the treatment course and/or non-treatment interval can be adjusted accordingly.

As indicated above, methods are provided herein for treating conditions, diseases, or disorders related to, associated with, or caused by cellular senescence, including age-related diseases and disorders in a subject in need thereof. A senescence-associated disease or disorder may also be called herein a senescent cell-associated disease or disorder. Senescence-associated diseases and disorders include, for example, cardiovascular diseases and disorders, inflammatory diseases and disorders, autoimmune diseases and disorders, pulmonary diseases and disorders, eye diseases and disorders, metabolic diseases and disorders, neurological diseases and disorders (e.g., neurodegenerative diseases and disorders); age-related diseases and disorders induced by senescence; skin conditions; age-related diseases; dermatological diseases and disorders; and transplant related diseases and disorders. A prominent feature of aging is a gradual loss of function, or degeneration that occurs at the molecular, cellular, tissue, and organismal levels. Age-related degeneration gives rise to well-recognized pathologies, such as sarcopenia, atherosclerosis and heart failure, osteoporosis, pulmonary insufficiency, renal failure, neurodegeneration (including macular degeneration, Alzheimer's disease, and Parkinson's disease), and many others. Although different mammalian species vary in their susceptibilities to specific age-related pathologies, collectively, age-related pathologies generally rise with approximately exponential kinetics beginning at about the mid-point of the species-specific life span (e.g., 50-60 years of age for humans) (see, e.g., Campisi (2013) Annu. Rev. Physiol. 75: 685-705; Naylor et al. (2013) Clin. Pharmacol. Ther. 93:105-16).

Examples of senescence-associated conditions, disorders, or diseases that may be treated by administering any one of the senolytic agents described herein (e.g., CRYAB inhibitors) according to the methods described herein include, cognitive diseases (e.g., mild cognitive impairment (MCI), Alzheimer's disease and other dementias; Huntington's disease); cardiovascular disease (e.g., atherosclerosis, cardiac diastolic dysfunction, aortic aneurysm, angina, arrhythmia, cardiomyopathy, congestive heart failure, coronary artery disease, myocardial infarction, endocarditis, hypertension, carotid artery disease, peripheral vascular diseases, cardiac stress resistance, cardiac fibrosis); metabolic diseases and disorders (e.g., obesity, diabetes, metabolic syndrome); motor function diseases and disorders (e.g., Parkinson's disease, motor neuron dysfunction (MND); Huntington's disease); cerebrovascular disease; emphysema; osteoarthritis; benign prostatic hypertrophy; pulmonary diseases (e.g., idiopathic pulmonary fibrosis, chronic obstructive pulmonary disease (COPD), emphysema, obstructive bronchiolitis, asthma); inflammatory/autoimmune diseases and disorders (e.g., osteoarthritis, eczema, psoriasis, osteoporosis, mucositis, transplantation related diseases and disorders); ophthalmic diseases or disorders (e.g., age-related macular degeneration, cataracts, glaucoma, vision loss, presbyopia); diabetic ulcer; metastasis; a chemotherapeutic side effect, a radiotherapy side effect; aging-related diseases and disorders (e.g., kyphosis, renal dysfunction, frailty, hair loss, hearing loss, muscle fatigue, skin conditions, sarcopenia, and herniated intervertebral disc) and other age-related diseases that are induced by senescence (e.g., diseases/disorders resulting from irradiation, chemotherapy, smoking tobacco, eating a high fat/high sugar diet, and environmental factors); wound healing; skin nevi; fibrotic diseases and disorders (e.g., cystic fibrosis, renal fibrosis, liver fibrosis, pulmonary fibrosis, oral submucous fibrosis, cardiac fibrosis, and pancreatic fibrosis). In certain embodiments, any one or more of the diseases or disorders described above or herein may be excluded.

In a more specific embodiment, methods are provided for treating a senescence-associated disease or disorder by killing senescent cells (i.e., established senescent cells) associated with the disease or disorder in a subject who has the disease or disorder by administering a senolytic agent (CRYAB inhibitor), wherein the disease or disorder is osteoarthritis; idiopathic pulmonary fibrosis; chronic obstructive pulmonary disease (COPD); or atherosclerosis.

In certain embodiments, subjects (i.e., patients, individuals (human or non-human animals)) who may benefit from use of the methods described herein that comprise administering a senolytic agent (e.g., a CRYAB inhibitor) include those who may also have a cancer. The subject treated by these methods may be considered to be in partial or complete remission (also called cancer remission). As discussed in detail herein, the senolytic agents (e.g., CRYAB inhibitors) for use in methods for selective killing of senescent cells are not intended to be used as a treatment for cancer, that is, in a manner that kills or destroys the cancer cells in a statistically significant manner Therefore, the methods disclosed herein do not encompass use of the senolytic agents in a manner that would be considered a primary therapy for the treatment of a cancer. Even though a senolytic agent, alone or with other chemotherapeutic or radiotherapy agents, are not used in a manner that is sufficient to be considered as a primary cancer therapy, the methods and senolytic agents described herein (e.g., CRYAB inhibitors) may be used in a manner (e.g., a short term course of therapy) that is useful for inhibiting metastases. In other certain embodiments, the subject to be treated with the senolytic agent does not have a cancer (i.e., the subject has not been diagnosed as having a cancer by a person skilled in the medical art).

### Cardiovascular Diseases and Disorders.

In another embodiment, the senescence-associated disease or disorder treated by the methods described herein is a cardiovascular disease. The cardiovascular disease may be any one or more of angina, arrhythmia, atherosclerosis, cardiomyopathy, congestive heart failure, coronary artery disease (CAD), carotid artery disease, endocarditis, heart attack (coronary thrombosis, myocardial infarction [MI]), high blood pressure/hypertension, aortic aneurysm, brain aneurysm, cardiac fibrosis, cardiac diastolic dysfunction, hypercholesterolemia/hyperlipidemia, mitral valve prolapse, peripheral vascular disease (e.g., peripheral artery disease (PAD)), cardiac stress resistance, and stroke.

In certain embodiments, methods are provided for treating senescence-associated cardiovascular disease that is associated with or caused by arteriosclerosis (i.e., hardening of the arteries). The cardiovascular disease may be any one or more of atherosclerosis (e.g., coronary artery disease (CAD) and carotid artery disease); angina, congestive heart failure, and peripheral vascular disease (e.g., peripheral artery disease (PAD)). The methods for treating a cardiovascular disease that is associated with or caused by arteriosclerosis may reduce the likelihood of occurrence of high blood pressure/hypertension, angina, stroke, and heart attack (i.e., coronary thrombosis, myocardial infarction (MI)). In certain embodiments, methods are provided for stabilizing atherosclerotic plaque(s) in a blood vessel (e.g., artery) of a subject, thereby reducing the likelihood of occurrence or delaying the occurrence of a thrombotic event, such as stroke or MI. In certain embodiments, these methods comprising administration of a senolytic agent to reduce (i.e., cause decrease of) the lipid content of an atherosclerotic plaque in a blood vessel (e.g., artery) of the subject and/or increase the fibrous cap thickness (i.e., cause an increase, enhance or promote thickening of the fibrous cap).

Atherosclerosis is characterized by patchy intimal plaques (atheromas) that encroach on the lumen of medium-sized and large arteries; the plaques contain lipids, inflammatory cells, smooth muscle cells, and connective tissue. Atherosclerosis can affect large and medium-sized arteries, including the coronary, carotid, and cerebral arteries, the aorta and its branches, and major arteries of the extremities. Atherosclerosis is characterized by patchy intimal plaques (atheromas) that encroach on the lumen of medium-sized and large arteries; the plaques contain lipids, inflammatory cells, smooth muscle cells, and connective tissue.

In one embodiment, methods are provided for inhibiting the formation of atherosclerotic plaques (or reducing, diminishing, causing decrease in formation of atherosclerotic plaques) by administering a senolytic agent (e.g., a CRYAB inhibitor). In other embodiments, methods are provided for reducing (decreasing, diminishing) the amount (i.e., level) of plaque. Reduction in the amount of plaque in a blood vessel (e.g., artery) may be determined, for example, by a decrease in surface area of the plaque, or by a decrease in the extent or degree (e.g., percent) of occlusion of a blood vessel (e.g., artery), which can be determined by angiography or other visualizing methods used in the cardiovascular art. Also provided herein are methods for increasing the stability (or improving, promoting, enhancing stability) of atherosclerotic plaques that are present in one or more blood vessels (e.g., one or more arteries) of a subject, which methods comprise administering to the subject any one of the senolytic agents described herein.

Atherosclerosis is often referred to as a “hardening” or furring of the arteries and is caused by the formation of multiple atheromatous plaques within the arteries. Atherosclerosis (also called arteriosclerotic vascular disease or ASVD herein and in the art) is a form of arteriosclerosis in which an artery wall thickens. Symptoms develop when growth or rupture of the plaque reduces or obstructs blood flow; and the symptoms may vary depending on which artery is affected. Atherosclerotic plaques may be stable or unstable. Stable plaques regress, remain static, or grow slowly, sometimes over several decades, until they may cause stenosis or occlusion. Unstable plaques are vulnerable to spontaneous erosion, fissure, or rupture, causing acute thrombosis, occlusion, and infarction long before they cause hemodynamically significant stenosis. Most clinical events result from unstable plaques, which do not appear severe on angiography; thus, plaque stabilization may be a way to reduce morbidity and mortality. Plaque rupture or erosion can lead to major cardiovascular events such as acute coronary syndrome and stroke (see, e.g., Du et al. (2014) BMC Cardiovascular Disorders 14: 83; Grimm et al. (2102) J. Cardiovasc. Magn. Res. 14: 80). Disrupted plaques were found to have a greater content of lipid, macrophages, and had a thinner fibrous cap than intact plaques (see, e.g., Felton et al.,) 1007_Arteriosclerosis, Thrombosis, and Vascular Biology 17: 1337-1345).

Atherosclerosis is a syndrome affecting arterial blood vessels due in significant part to a chronic inflammatory response of white blood cells in the walls of arteries. This is promoted by low-density lipoproteins (LDL, plasma proteins that carry cholesterol and triglycerides) in the absence of adequate removal of fats and cholesterol from macrophages by functional high-density lipoproteins (HDL). The earliest visible lesion of atherosclerosis is the “fatty streak,” which is an accumulation of lipid-laden foam cells in the intimal layer of the artery. The hallmark of atherosclerosis is atherosclerotic plaque, which is an evolution of the fatty streak and has three major components: lipids (e.g., cholesterol and triglycerides); inflammatory cells and smooth muscle cells; and a connective tissue matrix that may contain thrombi in various stages of organization and calcium deposits. Within the outer-most and oldest plaque, calcium and other crystallized components (e.g., microcalcification) from dead cells can be found. Microcalcification and properties related thereto are also thought to contribute to plaque instability by increasing plaque stress (see, e.g., Bluestein et al., J. Biomech. 41(5):1111-18 (2008); Cilla et al., Journal of Engineering in Medicine 227:588-99 (2013)). Fatty streaks reduce the elasticity of the artery walls, but may not affect blood flow for years because the artery muscular wall accommodates by enlarging at the locations of plaque. Lipid-rich atheromas are at increased risk for plaque rupture and thrombosis (see, e.g., Felton et al., supra; Fuster et al., J. Am. Coll. Cardiol. 46:1209-18 (2005)). Reports have found that of all plaque components, the lipid core exhibits the highest thrombogenic activity (see, e.g., Fernandez-Ortiz et al., J. Am. Coll. Cardiol. 23:1562-69 (1994)). Within major arteries in advanced disease, the wall stiffening may also eventually increase pulse pressure.

A vulnerable plaque that may lead to a thrombotic event (stroke or MI) and is sometimes described as a large, soft lipid pool covered by a thin fibrous cap (see, e.g., Li et al., Stroke 37:1195-99 (2006); Trivedi et al., Neuroradiology 46:738-43 (2004)). An advanced characteristic feature of advance atherosclerotic plaque is irregular thickening of the arterial intima by inflammatory cells, extracellular lipid (atheroma) and fibrous tissue (sclerosis) (see, e.g., Newby et al., Cardiovasc. Res. 345-60 (1999)). Fibrous cap formation is believed to occur from the migration and proliferation of vascular smooth muscle cells and from matrix deposition (see, e.g., Ross, Nature 362:801-809 (1993); Sullivan et al., J. Angiology at dx.doi.org/10.1155/2013/592815 (2013)). A thin fibrous cap contributes to instability of the plaque and to increased risk for rupture (see, e.g., Li et al., supra).

Both proinflammatory macrophages (M1) and anti-inflammatory macrophages (M2) can be found in arteriosclerotic plaque. The contribution of both types of cells to plaque instability is a subject of active investigation, with results suggesting that an increased level of the M1 type versus the M2 type correlates with increased instability of plaque (see, e.g., Medbury et al., Int. Angiol. 32:74-84 (2013); Lee et al., Am. J. Clin. Pathol. 139:317-22 (2013); Martinet et al., Cir. Res. 751-53 (2007)).

Subjects suffering from cardiovascular disease can be identified using standard diagnostic methods known in the art for cardiovascular disease. Generally, diagnosis of atherosclerosis and other cardiovascular disease is based on symptoms (e.g., chest pain or pressure (angina), numbness or weakness in arms or legs, difficulty speaking or slurred speech, drooping muscles in face, leg pain, high blood pressure, kidney failure and/or erectile dysfunction), medical history, and/or physical examination of a patient. Diagnosis may be confirmed by angiography, ultrasonography, or other imaging tests. Subjects at risk of developing cardiovascular disease include those having any one or more of predisposing factors, such as a family history of cardiovascular disease and those having other risk factors (i.e., predisposing factors) such as high blood pressure, dyslipidemia, high cholesterol, diabetes, obesity and cigarette smoking, sedentary lifestyle, and hypertension. In a certain embodiment, the cardiovascular disease that is a senescence cell associated disease/disorder is atherosclerosis.

The effectiveness of one or more senolytic agents (e.g., CRYAB inhibitors) for treating or preventing (i.e., reducing or decreasing the likelihood of developing or occurrence of) a cardiovascular disease (e.g., atherosclerosis) can readily be determined by a person skilled in the medical and clinical arts. One or any combination of diagnostic methods, including physical examination, assessment and monitoring of clinical symptoms, and performance of analytical tests and methods described herein and practiced in the art (e.g., angiography, electrocardiography, stress test, non-stress test), may be used for monitoring the health status of the subject. The effects of the treatment of a senolytic agent or pharmaceutical composition comprising same can be analyzed using techniques known in the art, such as comparing symptoms of patients suffering from or at risk of cardiovascular disease that have received the treatment with those of patients without such a treatment or with placebo treatment.

### Inflammatory and Autoimmune Diseases and Disorders.

In certain embodiments, a senescence-associated disease or disorder is an inflammatory disease or disorder, such as by way of non-limiting example, osteoarthritis, that may be treated or prevented (i.e., likelihood of occurrence is reduced) according to the methods described herein that comprise administration of a senolytic agent. Other inflammatory or autoimmune diseases or disorders that may be treated by administering a senolytic agent such as the CRYAB inhibitors described herein include osteoporosis, psoriasis, oral mucositis, rheumatoid arthritis, inflammatory bowel disease, eczema, kyphosis, herniated intervertebral disc, and the pulmonary diseases, COPD and idiopathic pulmonary fibrosis.

Osteoarthritis degenerative joint disease is characterized by fibrillation of the cartilage at sites of high mechanical stress, bone sclerosis, and thickening of the synovium and the joint capsule. Fibrillation is a local surface disorganization involving splitting of the superficial layers of the cartilage. The early splitting is tangential with the cartilage surface, following the axes of the predominant collagen bundles. Collagen within the cartilage becomes disorganized, and proteoglycans are lost from the cartilage surface. In the absence of protective and lubricating effects of proteoglycans in a joint, collagen fibers become susceptible to degradation, and mechanical destruction ensues. Predisposing risk factors for developing osteoarthritis include increasing age, obesity, previous joint injury, overuse of the joint, weak thigh muscles, and genetics. It is a common cause of chronic disability in the elderly. Symptoms of osteoarthritis include sore or stiff joints, particularly the hips, knees, and lower back, after inactivity or overuse; stiffness after resting that goes away after movement; and pain that is worse after activity or toward the end of the day. Osteoarthritis may also affect the neck, small finger joints, the base of the thumb, ankle, and big toe.

Chronic inflammation is thought to be the main age-related factor that contributes to osteoarthritis. In combination with aging, joint overuse and obesity appear to promote osteoarthritis.

Unexpectedly, by selectively killing senescent cells a senolytic agent (e.g., CRYAB inhibitor) prevents (i.e., reduces the likelihood of occurrence), reduces or inhibits loss or erosion of proteoglycan layers in a joint, reduces inflammation in the affected joint, and promotes (i.e., stimulates, enhances, induces) production of collagen (e.g., type 2 collagen). Removal of senescent cells causes a reduction in the amount (i.e., level) of inflammatory cytokines, such as IL-6, produced in a joint and reduction of inflammation. Methods are provided herein for treating osteoarthritis, for selectively killing senescent cells in an osteoarthritic joint of a subject, and/or inducing collagen (such as Type 2 collagen) production in the joint of a subject in need thereof by administering at least one senolytic agent (which may be combined with at least one pharmaceutically acceptable excipient to form a pharmaceutical composition) to the subject. A senolytic agent also may be used for decreasing (inhibiting, reducing) production of metalloproteinase 13 (MMP-13), which degrades collagen in a joint, and for restoring proteoglycan layer or inhibiting loss and/or degradation of the proteoglycan layer. Treatment with the senolytic agent thereby also prevents (i.e., reduces likelihood of occurrence of), inhibits, or decreases erosion, or slows (i.e., decreases rate) erosion of the bone. As described in detail herein, in certain embodiments, the senolytic agent is administered directly to an osteoarthritic joint (e.g., by intra-articular, topical, transdermal, intradermal, or subcutaneous delivery). Treatment with a senolytic agent can also restore, improve, or inhibit deterioration of strength of a joint. In addition, the methods comprising administering a senolytic agent can reduce joint pain and are therefore useful for pain management of osteoarthritic joints.

The effectiveness of one or more senolytic agents (e.g., CRYAB inhibitors) for treatment or prophylaxis of osteoarthritis in a subject and monitoring of a subject who receives one or more senolytic agents can readily be determined by a person skilled in the medical and clinical arts. One or any combination of diagnostic methods, including physical examination (such as determining tenderness, swelling or redness of the affected joint), assessment and monitoring of clinical symptoms (such as pain, stiffness, mobility), and performance of analytical tests and methods described herein and practiced in the art (e.g., determining the level of inflammatory cytokines or chemokines; X-ray images to determine loss of cartilage as shown by a narrowing of space between the bones in a joint; magnetic resonance imaging (MRI), providing detailed images of bone and soft tissues, including cartilage), may be used for monitoring the health status of the subject. The effects of the treatment of one or more senolytic agents can be analyzed by comparing symptoms of patients suffering from or at risk of an inflammatory disease or disorder, such as osteoarthritis, who have received the treatment with those of patients who have not received such a treatment or who have received a placebo treatment.

In certain embodiments, senolytic agents (e.g., CRYAB inhibitors) may be used for treating and/or preventing (i.e., decreasing or reducing the likelihood of occurrence) rheumatoid arthritis (RA). Dysregulation of innate and adaptive immune responses characterize rheumatoid arthritis (RA), which is an autoimmune disease the incidence of which increases with age. Rheumatoid arthritis is a chronic inflammatory disorder that typically affects the small joints in hands and feet. Whereas osteoarthritis results from, at least in part, wear and tear of a joint, rheumatoid arthritis affects the lining of joints, resulting in a painful swelling that can lead to bone erosion and joint deformity. RA can sometimes also affect other organs of the body, such as the skin, eyes, lungs and blood vessels. RA can occur in a subject at any age; however, RA usually begins to develop after age 40. The disorder is much more common in women. In certain embodiments of the methods described herein, RA is excluded.

Chronic inflammation may also contribute to other age-related or aging related diseases and disorders, such as kyphosis and osteoporosis. Kyphosis is a severe curvature in the spinal column, and it is frequently seen with normal and premature aging (see, e.g., Katzman et al. (2010) J. Orthop. Sports Phys. Ther. 40: 352-360). Age-related kyphosis often occurs after osteoporosis weakens spinal bones to the point that they crack and compress. A few types of kyphosis target infants or teens. Severe kyphosis can affect lungs, nerves, and other tissues and organs, causing pain and other problems. Kyphosis has been associated with cellular senescence. Characterizing the capability of a senolytic agent for treating kyphosis may be determined in pre-clinical animal models used in the art. By way of example, TTD mice develop kyphosis (see, e.g., de Boer et al. (2002) Science 296: 1276-1279); other mice that may be used include BubR1^{H/H} mice, which are also known to develop kyphosis (see, e.g., Baker et al. (2011) Nature 479: 232-36). Kyphosis formation is visually measured over time. The level of senescent cells decreased by treatment with the senolytic agent can be determined by detecting the presence of one or more senescent cell associated markers such as by SA-β-Gal staining.

Osteoporosis is a progressive bone disease that is characterized by a decrease in bone mass and density that may lead to an increased risk of fracture. Bone mineral density (BMD) is reduced, bone microarchitecture deteriorates, and the amount and variety of proteins in bone are altered. Osteoporosis is typically diagnosed and monitored by a bone mineral density test. Post-menopausal women or women who have reduced estrogen are most at risk. While both men and women over 75 are at risk, women are twice as likely to develop osteoporosis than men. The level of senescent cells decreased by treatment with the senolytic agent can be determined by detecting the presence of one or more senescent cell associated markers such as by SA-β-Gal staining.

In still other embodiments, an inflammatory/autoimmune disorder that may be treated or prevented (i.e., likelihood of occurrence is reduced) with the senolytic agents described herein (e.g., CRYAB inhibitors) includes irritable bowel syndrome (IBS) and inflammatory bowel diseases, such as ulcerative colitis and Crohn's disease. Inflammatory bowel disease (IBD) involves chronic inflammation of all or part of the digestive tract. In addition to life-threatening complications arising from IBD, the disease can be painful and debilitating. Ulcerative colitis is an inflammatory bowel disease that causes long-lasting inflammation in part of the digestive tract. Symptoms usually develop over time, rather than suddenly. Ulcerative colitis usually affects only the innermost lining of the large intestine (colon) and rectum. Crohn's disease is an inflammatory bowel disease that causes inflammation anywhere along the lining of your digestive tract, and often extends deep into affected tissues. This can lead to abdominal pain, severe diarrhea, and malnutrition. The inflammation caused by Crohn's disease can involve different areas of the digestive tract. Diagnosis and monitoring of the diseases is performed according to methods and diagnostic tests routinely practiced in the art, including blood tests, colonoscopy, flexible sigmoidoscopy, barium enema, CT scan, MRI, endoscopy, and small intestine imaging.

In other embodiments, the methods described herein may be useful for treating a subject who has herniated intervertebral discs. Subjects with these herniated discs exhibit elevated presence of cell senescence in the blood and in vessel walls (see e.g., Roberts et al. (2006) Eur. Spine J. 15 Suppl 3: S312-316). Symptoms of a herniated intervertebral disc may include pain, numbness or tingling, or weakness in an arm or leg. Increased levels of proinflammatory molecules and matrix metalloproteases are also found in aging and degenerating discs tissues, suggesting a role for senescence cells (see e.g., Chang-Qing et al. (2007) Ageing Res. Rev. 6: 247-61). Animal models may be used to characterize the effectiveness of a senolytic agent in treating herniated intervertebral discs; degeneration of the intervertebral disc is induced in mice by compression and disc strength evaluated (see e.g., Lotz et al. (1998) Spine (Philadelphia Pa. 1976). 23:2493-506).

Other inflammatory or autoimmune diseases that may be treated or prevented (i.e., likelihood of occurrence is reduced) by using a senolytic agent described herein (e.g., CRYAB inhibitor) include eczema, psoriasis, osteoporosis, and pulmonary diseases (e.g., chronic obstructive pulmonary disease (COPD), idiopathic pulmonary fibrosis (IPF), asthma), inflammatory bowel disease, and mucositis (including oral mucositis, which in some instances is induced by radiation). Certain fibrosis or fibrotic conditions of organs such as renal fibrosis, liver fibrosis, pancreatic fibrosis, cardiac fibrosis, skin wound healing, and oral submucous fibrosis may be treated with a senolytic agent.

In certain embodiments, the senescent cell associated disorder is an inflammatory disorder of the skin, such as by way of a non-limiting examples, psoriasis and eczema that may be treated or prevented (i.e., likelihood of occurrence is reduced) according to the methods described herein that comprise administration of a senolytic agent. Psoriasis is characterized by abnormally excessive and rapid growth of the epidermal layer of the skin. A diagnosis of psoriasis is usually based on the appearance of the skin. Skin characteristics typical for psoriasis are scaly red plaques, papules, or patches of skin that may be painful and itch. In psoriasis, cutaneous and systemic overexpression of various proinflammatory cytokines is observed such as IL-6, a key component of the SASP. Eczema is an inflammation of the skin that is characterized by redness, skin swelling, itching and dryness, crusting, flaking, blistering, cracking, oozing, or bleeding. The effectiveness of senolytic agents for treatment of psoriasis and eczema and monitoring of a subject who receives such a senolytic agent can be readily determined by a person skilled in the medical or clinical arts. One or any combination of diagnostic methods, including physical examination (such as skin appearance), assessment of monitoring of clinical symptoms (such as itching, swelling, and pain), and performance of analytical tests and methods described herein and practiced in the art (i.e., determining the level of pro-inflammatory cytokines).

Other immune disorders or conditions that may be treated or prevented (i.e., likelihood of occurrence is reduced) with a senolytic agent include conditions resulting from a host immune response to an organ transplant (e.g., kidney, bone marrow, liver, lung, or heart transplant), such as rejection of the transplanted organ. The senolytic agent (e.g., CRYAB inhibitor) may be used for treating or reducing the likelihood of occurrence of graft-vs-host disease.

### Pulmonary Diseases and Disorders.

In one embodiment, methods are provided for treating or preventing (i.e., reducing the likelihood of occurrence of) a senescence-associated disease or disorder that is a pulmonary disease or disorder by killing senescent cells (i.e., established senescent cells) associated with the disease or disorder in a subject who has the disease or disorder by administering a senolytic agent described herein (e.g., a CRYAB inhibitor). Senescence associated pulmonary diseases and disorders include, for example, idiopathic pulmonary fibrosis (IPF), chronic obstructive pulmonary disease (COPD), asthma, cystic fibrosis, bronchiectasis, and emphysema.

COPD is a lung disease defined by persistently poor airflow resulting from the breakdown of lung tissue (emphysema) and the dysfunction of the small airways (obstructive bronchiolitis). Primary symptoms of COPD include shortness of breath, wheezing, chest tightness, chronic cough, and excess sputum production. Elastase from cigarette smoke-activated neutrophils and macrophages disintegrates the extracellular matrix of alveolar structures, resulting in enlarged air spaces and loss of respiratory capacity (see, e.g., Shapiro et al., Am. J. Respir. Cell Mol. Biol. 32, 367-372 (2005)). COPD is most commonly caused by tobacco smoke (including cigarette smoke, cigar smoke, secondhand smoke, pipe smoke), occupational exposure (e.g., exposure to dust, smoke or fumes), and pollution, occurring over decades thereby implicating aging as a risk factor for developing COPD.

The processes involved in causing lung damage include, for example, oxidative stress produced by the high concentrations of free radicals in tobacco smoke; cytokine release due to inflammatory response to irritants in the airway; and impairment of anti-protease enzymes by tobacco smoke and free radicals, allowing proteases to damage the lungs. Genetic susceptibility can also contribute to the disease. In about 1% of people with COPD, the disease results from a genetic disorder that causes low level production of alpha-1-antitrypsin in the liver. The enzyme is normally secreted into the bloodstream to help protect the lungs.

Pulmonary fibrosis is a chronic and progressive lung disease characterized by stiffening and scarring of the lung, which may lead to respiratory failure, lung cancer, and heart failure. Fibrosis is associated with repair of epithelium. Fibroblasts are activated, production of extracellular matrix proteins is increased, and transdifferentiation to contractile myofibroblasts contribute to wound contraction. A provisional matrix plugs the injured epithelium and provides a scaffold for epithelial cell migration, involving an epithelial-mesenchymal transition (EMT). Blood loss associated with epithelial injury induces platelet activation, production of growth factors, and an acute inflammatory response. Normally, the epithelial barrier heals and the inflammatory response resolves. However, in fibrotic disease the fibroblast response continues, resulting in unresolved wound healing. Formation of fibroblastic foci is a feature of the disease, reflecting locations of ongoing fibrogenesis. As the name connotes, the etiology of IPF is unknown. The involvement of cellular senescence in IPF is suggested by the observations that the incidence of the disease increases with age and that lung tissue in IPF patients is enriched for SA-β-Gal-positive cells and contains elevated levels of the senescence marker p21 (see, e.g., Minagawa et al., Am. J. Physiol. Lung Cell. Mol. Physiol. 300:L391-L401 (2011); see also, e.g., Naylor et al., supra). Short telomeres are a risk factor common to both IPF and cellular senescence (see, e.g., Alder et al., Proc. Natl. Acad. Sci. USA 105:13051-56 (2008)). Without wishing to be bound by theory, the contribution of cellular senescence to IPF is suggested by the report that SASP components of senescent cells, such as IL-6, IL-8, and IL-1β, promotes fibroblast-to-myofibroblast differentiation and epithelial-mesenchymal transition, resulting in extensive remodeling of the extracellular matrix of the alveolar and interstitial spaces (see, e.g., Minagawa et al., supra).

Subjects at risk of developing pulmonary fibrosis include those exposed to environmental or occupational pollutants, such as asbestosis and silicosis; who smoke cigarettes; having some typical connective tissue diseases such as rheumatoid arthritis, SLE and scleroderma; having other diseases that involve connective tissue, such as sarcoidosis and Wegener's granulomatosis; having infections; taking certain medications (e.g., amiodarone, bleomycin, busufan, methotrexate, and nitrofurantoin); those subject to radiation therapy to the chest; and those whose family member has pulmonary fibrosis.

Symptoms of COPD may include any one of shortness of breath, especially during physical activities; wheezing; chest tightness; having to clear your throat first thing in the morning because of excess mucus in the lungs; a chronic cough that produces sputum that may be clear, white, yellow or greenish; blueness of the lips or fingernail beds (cyanosis); frequent respiratory infections; lack of energy; unintended weight loss (observed in later stages of disease). Subjects with COPD may also experience exacerbations, during which symptoms worsen and persist for days or longer. Symptoms of pulmonary fibrosis are known in the art and include shortness of breath, particularly during exercise; dry, hacking cough; fast, shallow breathing; gradual unintended weight loss; tiredness; aching joints and muscles; and clubbing (widening and rounding of the tips of the fingers or toes).

Subjects suffering from COPD or pulmonary fibrosis can be identified using standard diagnostic methods routinely practiced in the art. Monitoring the effect of one or more senolytic agents administered to a subject who has or who is at risk of developing a pulmonary disease may be performed using the methods typically used for diagnosis. Generally, one or more of the following exams or tests may be performed: physical exam, patient's medical history, patient's family's medical history, chest X-ray, lung function tests (such as spirometry), blood test (e.g., arterial blood gas analysis), bronchoalveolar lavage, lung biopsy, CT scan, and exercise testing.

Other pulmonary diseases or disorders that may be treated by using a senolytic agent described herein (e.g., a CRYAB inhibitor) include, for example, emphysema, asthma, bronchiectasis, and cystic fibrosis (see, e.g., Fischer et al., Am J Physiol Lung Cell Mol Physiol. 304(6):L394-400 (2013)). These diseases may also be exacerbated by tobacco smoke (including cigarette smoke, cigar smoke, secondhand smoke, pipe smoke), occupational exposure (e.g., exposure to dust, smoke or fumes), infection, and/or pollutants that induce cells into senescence and thereby contribute to inflammation. Emphysema is sometimes considered as a subgroup of COPD.

Bronchiectasis results from damage to the airways that causes them to widen and become flabby and scarred. Bronchiectasis usually is caused by a medical condition that injures the airway walls or inhibits the airways from clearing mucus. Examples of such conditions include cystic fibrosis and primary ciliary dyskinesia (PCD). When only one part of the lung is affected, the disorder may be caused by a blockage rather than a medical condition.

The methods described herein for treating or preventing (i.e., reducing the likelihood of occurrence of) a senescence associated pulmonary disease or disorder may also be used for treating a subject who is aging and has loss (or degeneration) of pulmonary function (i.e., declining or impaired pulmonary function compared with a younger subject) and/or degeneration of pulmonary tissue. The respiratory system undergoes various anatomical, physiological and immunological changes with age. The structural changes include chest wall and thoracic spine deformities that can impair the total respiratory system compliance resulting in increased effort to breathe. The respiratory system undergoes structural, physiological, and immunological changes with age. An increased proportion of neutrophils and lower percentage of macrophages can be found in bronchoalveolar lavage (BAL) of older adults compared with younger adults. Persistent low grade inflammation in the lower respiratory tract can cause proteolytic and oxidant-mediated injury to the lung matrix resulting in loss of alveolar unit and impaired gas exchange across the alveolar membrane seen with aging. Sustained inflammation of the lower respiratory tract may predispose older adults to increased susceptibility to toxic environmental exposure and accelerated lung function decline. (See, for example, Sharma et al., Clinical Interventions in Aging 1:253-60 (2006)). Oxidative stress exacerbates inflammation during aging (see, e.g., Brod, Inflamm Res 2000; 49:561-570; Hendel et al., Cell Death and Differentiation (2010) 17:596-606). Alterations in redox balance and increased oxidative stress during aging precipitate the expression of cytokines, chemokines, and adhesion molecules, and enzymes (see, e.g., Chung et al., Ageing Res Rev 2009; 8:18-30). Constitutive activation and recruitment of macrophages, T cells, and mast cells foster release of proteases leading to extracellular matrix degradation, cell death, remodeling, and other events that can cause tissue and organ damage during chronic inflammation (see, e.g., Demedts et al., Respir Res 2006; 7: 53-63). By administering a senolytic agent to an aging subject (which includes a middle-aged adult who is asymptomatic), the decline in pulmonary function may be decelerated or inhibited by killing and removing senescent cells from the respiratory tract.

The effectiveness of a senolytic agent can readily be determined by a person skilled in the medical and clinical arts. One or any combination of diagnostic methods, including physical examination, assessment and monitoring of clinical symptoms, and performance of analytical tests and methods described herein, may be used for monitoring the health status of the subject. The effects of the treatment of a senolytic agent or pharmaceutical composition comprising the agent can be analyzed using techniques known in the art, such as comparing symptoms of patients suffering from or at risk of the pulmonary disease that have received the treatment with those of patients without such a treatment or with placebo treatment. In addition, methods and techniques that evaluate mechanical functioning of the lung, for example, techniques that measure lung capacitance, elastance, and airway hypersensitivity may be performed. To determine lung function and to monitor lung function throughout treatment, any one of numerous measurements may be obtained, expiratory reserve volume (ERV), forced vital capacity (FVC), forced expiratory volume (FEV) (e.g., FEV in one second, FEV1), FEV1/FEV ratio, forced expiratory flow 25% to 75%, and maximum voluntary ventilation (MVV), peak expiratory flow (PEF), slow vital capacity (SVC). Total lung volumes include total lung capacity (TLC), vital capacity (VC), residual volume (RV), and functional residual capacity (FRC). Gas exchange across alveolar capillary membrane can be measured using diffusion capacity for carbon monoxide (DLCO). Peripheral capillary oxygen saturation (SpO₂) can also be measured; normal oxygen levels are typically between 95% and 100%. An SpO₂ level below 90% suggests the subject has hypoxemia. Values below 80% are considered critical and requiring intervention to maintain brain and cardiac function and avoid cardiac or respiratory arrest.

### Neurological Diseases and Disorders.

Senescence-associated diseases or disorders treatable by administering a senolytic agent described herein include neurological diseases or disorders. Such senescence-associated diseases and disorders include Parkinson's disease, Alzheimer's disease (and other dementias), motor neuron dysfunction (MND), mild cognitive impairment (MCI), Huntington's disease, and diseases and disorders of the eyes, such as age-related macular degeneration. Other diseases of the eye that are associated with increasing age are glaucoma, vision loss, presbyopia, and cataracts.

Parkinson's disease (PD) is the second most common neurodegenerative disease. It is a disabling condition of the brain characterized by slowness of movement (bradykinesia), shaking, stiffness, and in the later stages, loss of balance. Many of these symptoms are due to the loss of certain nerves in the brain, which results in a lack of dopamine. This disease is characterized by neurodegeneration, such as the loss of about 50% to 70% of the dopaminergic neurons in the substantia nigra pars compacta, a profound loss of dopamine in the striatum, and/or the presence of intracytoplasmic inclusions (Lewy bodies), which are composed mainly of alpha-synuclein and ubiquitin. Parkinson's disease also features locomotor deficits, such as tremor, rigidity, bradykinesia, and/or postural instability. Subjects at risk of developing Parkinson's disease include those having a family history of Parkinson's disease and those exposed to pesticides (e.g., rotenone or paraquat), herbicides (e.g., agent orange), or heavy metals. Senescence of dopamine-producing neurons is thought to contribute to the observed cell death in PD through the production of reactive oxygen species (see, e.g., Cohen et al., J. Neural Transm. Suppl. 19:89-103 (1983)); therefore, the methods and senolytic agents described herein are useful for treatment and prophylaxis of Parkinson's disease.

Methods for detecting, monitoring or quantifying neurodegenerative deficiencies and/or locomotor deficits associated with Parkinson's disease are known in the art, such as histological studies, biochemical studies, and behavioral assessment (see, e.g., U.S. Application Publication No. 2012/0005765). Symptoms of Parkinson's disease are known in the art and include, but are not limited to, difficulty starting or finishing voluntary movements, jerky, stiff movements, muscle atrophy, shaking (tremors), and changes in heart rate, but normal reflexes, bradykinesia, and postural instability. There is a growing recognition that people diagnosed with Parkinson's disease may have cognitive impairment, including mild cognitive impairment, in addition to their physical symptoms.

Alzheimer's disease (AD) is a neurodegenerative disease that shows a slowly progressive mental deterioration with failure of memory, disorientation, and confusion, leading to profound dementia. Age is the single greatest predisposing risk factor for developing AD, which is the leading cause of dementia in the elderly (see, e.g., Hebert, et al., Arch. Neural. 60:1119-1122 (2003)). Early clinical symptoms show remarkable similarity to mild cognitive impairment (see below). As the disease progresses, impaired judgment, confusion, behavioral changes, disorientation, and difficulty in walking and swallowing occur.

Alzheimer's disease is characterized by the presence of neurofibrillary tangles and amyloid (senile) plaques in histological specimens. The disease predominantly involves the limbic and cortical regions of the brain. The argyrophilic plaques containing the amyloidogenic A3 fragment of amyloid precursor protein (APP) are scattered throughout the cerebral cortex and hippocampus. Neurofibrillary tangles are found in pyramidal neurons predominantly located in the neocortex, hippocampus, and nucleus basalis of Meynert. Other changes, such as granulovacuolar degeneration in the pyramidal cells of the hippocampus, and neuron loss and gliosis in the cortex and hippocampus, are observed. Subjects at risk of developing Alzheimer's disease include those of advanced age, those with a family history of Alzheimer's disease, those with genetic risk genes (e.g., ApoE4) or deterministic gene mutations (e.g., APP, PS1, or PS2), and those with history of head trauma or heart/vascular conditions (e.g., high blood pressure, heart disease, stroke, diabetes, high cholesterol).

A number of behavioral and histopathological assays are known in the art for evaluating Alzheimer's disease phenotype, for characterizing therapeutic agents, and assessing treatment. Histological analyses are typically performed postmortem. Histological analysis of Aβ levels may be performed using Thioflavin-S. Congo red, or anti-Aβ staining (e.g., 4G8, 10D5, or 6E10 antibodies) to visualize Aβ deposition on sectioned brain tissues (see, e.g., Holcomb et al., 1998, Nat. Med. 4:97-100; Borchelt et al., 1997, Neuron 19:939-945; Dickson et al., 1988, Am. J. Path. 132:86-101). In vivo methods of visualizing Aβ deposition in transgenic mice have been also described. BSB ((trans, trans)-1-bromo-2,5-bis-(3-hydroxycarbonyl-4-hydroxy)styrylbenzene) and PET tracer ¹¹C-labelled Pittsburgh Compound-B (PIB) bind to AP plaques (see, e.g., Skovronsky et al., 2000, Proc. Natl. Acad. Sci. USA 97:7609-7614; Klunk et al., 2004, Ann. Neurol. 55:306-319). ¹⁹F-containing amyloidophilic Congo red-type compound FSB ((E,E)-1-fluoro-2,5-bis-(3-hydroxycarbonyl-4-hydroxy)styrylbenzene) allows visualization of Aβ plaques by MRI (see, e.g., Higuchi et al., 2005, Nature Neurosci. 8:527-533). Radiolabeled, putrescine-modified amyloid-beta peptide labels amyloid deposits in vivo in a mouse model of Alzheimer's disease (see, e.g., Wengenack et al., 2000, Nat. Biotechnol. 18:868-872).

Increased glial fibrillary acidic protein (GFAP) by astrocytes is a marker for astroglial activation and gliosis during neurodegeneration. AP plaques are associated with GFAP-positive activated astrocytes, and may be visualized via GFAP staining (see, e.g., Nagele et al. 2004, Neurobiol. Aging 25:663-674; Mandybur et al., 1990, Neurology 40:635-639; Liang et al., 2010, J. Biol. Chem. 285:27737-27744). Neurofibrillary tangles may be identified by immunohistochemistry using thioflavin-S fluorescent microscopy and Gallyas silver stains (see, e.g., Gotz et al., 2001, J. Biol. Chem. 276:529-534; U.S. Pat. No. 6,664,443). Axon staining with electron microscopy and axonal transport studies may be used to assess neuronal degeneration (see, e.g., Ishihara et al., 1999, Neuron 24:751-762).

Subjects suffering from Alzheimer's disease can be identified using standard diagnostic methods known in the art for Alzheimer's disease. Generally, diagnosis of Alzheimer's disease is based on symptoms (e.g., progressive decline in memory function, gradual retreat from and frustration with normal activities, apathy, agitation or irritability, aggression, anxiety, sleep disturbance, dysphoria, aberrant motor behavior, disinhibition, social withdrawal, decreased appetite, hallucinations, dementia), medical history, neuropsychological tests, neurological and/or physical examination of a patient. Cerebrospinal fluid may also be for tested for various proteins that have been associated with Alzheimer pathology, including tau, amyloid beta peptide, and AD7C-NTP. Genetic testing is also available for early-onset familial Alzheimer disease (eFAD), an autosomal-dominant genetic disease. Clinical genetic testing is available for individuals with AD symptoms or at-risk family members of patients with early-onset disease. In the U.S., mutations for PS2, and APP may be tested in a clinical or federally approved laboratory under the Clinical Laboratory Improvement Amendments. A commercial test for PS1 mutations is also available (Elan Pharmaceuticals).

The effectiveness of one or more senolytic agents described herein (e.g., a CRYAB inhibitor) and monitoring of a subject who receives one or more senolytic agents can readily be determined by a person skilled in the medical and clinical arts. One or any combination of diagnostic methods, including physical examination, assessment and monitoring of clinical symptoms, and performance of analytical tests and methods described herein, may be used for monitoring the health status of the subject. The effects of administering one or more senolytic agents can be analyzed using techniques known in the art, such as comparing symptoms of patients suffering from or at risk of Alzheimer's disease that have received the treatment with those of patients without such a treatment or with placebo treatment.

### Mild Cognitive Impairment (MCI).

MCI is a brain-function syndrome involving the onset and evolution of cognitive impairments beyond those expected based on age and education of the individual, but which are not significant enough to interfere with this individual's daily activities. MCI is an aspect of cognitive aging that is considered to be a transitional state between normal aging and the dementia into which it may convert (see, Pepeu, Dialogues in Clinical Neuroscience 6:369-377, 2004). MCI that primarily affects memory is known as “amnestic MCI.” A person with amnestic MCI may start to forget important information that he or she would previously have recalled easily, such as recent events. Amnestic MCI is frequently seen as prodromal stage of Alzheimer's disease. MCI that affects thinking skills other than memory is known as “non-amnestic MCI.” This type of MCI affect thinking skills such as the ability to make sound decisions, judge the time or sequence of steps needed to complete a complex task, or visual perception. Individuals with non-amnestic MCI are believed to be more likely to convert to other types of dementias (e.g., dementia with Lewy bodies).

Persons in the medical art have a growing recognition that people diagnosed with Parkinson's disease may have MCI in addition to their physical symptoms. Recent studies show 20-30% of people with Parkinson's disease have MCI, and that their MCI tends to be non-amnestic. Parkinson's disease patients with MCI sometimes go on to develop full blown dementia (Parkinson's disease with dementia).

Methods for detecting, monitoring, quantifying or assessing neuropathological deficiencies associated with MCI are known in the art, including astrocyte morphological analyses, release of acetylcholine, silver staining for assessing neurodegeneration, and PiB PET imaging to detect beta amyloid deposits (see, e.g., U.S. Application Publication No. 2012/0071468; Pepeu, 2004, supra). Methods for detecting, monitoring, quantifying or assessing behavioral deficiencies associated with MCI are also known in the art, including eight-arm radial maze paradigm, non-matching-to-sample task, allocentric place determination task in a water maze, Morris maze test, visuospatial tasks, and delayed response spatial memory task, olfactory novelty test (see, id.).

### Motor Neuron Dysfunction (MND).

MND is a group of progressive neurological disorders that destroy motor neurons, the cells that control essential voluntary muscle activity such as speaking, walking, breathing and swallowing. It is classified according to whether degeneration affects upper motor neurons, lower motor neurons, or both. Examples of MNDs include, but are not limited to Amyotrophic Lateral Sclerosis (ALS), also known as Lou Gehrig's Disease, progressive bulbar palsy, pseudobulbar palsy, primary lateral sclerosis, progressive muscular atrophy, lower motor neuron disease, and spinal muscular atrophy (SMA) (e.g., SMA1 also called Werdnig-Hoffmann Disease, SMA2, SMAβ also called Kugelberg-Welander Disease, and Kennedy's disease), post-polio syndrome, and hereditary spastic paraplegia. In adults, the most common MND is amyotrophic lateral sclerosis (ALS), which affects both upper and lower motor neurons. It can affect the arms, legs, or facial muscles. Primary lateral sclerosis is a disease of the upper motor neurons, while progressive muscular atrophy affects only lower motor neurons in the spinal cord. In progressive bulbar palsy, the lowest motor neurons of the brain stem are most affected, causing slurred speech and difficulty chewing and swallowing. There are almost always mildly abnormal signs in the arms and legs. Patients with MND exhibit a phenotype of Parkinson's disease (e.g., having tremor, rigidity, bradykinesia, and/or postural instability). Methods for detecting, monitoring or quantifying locomotor and/or other deficits associated with Parkinson's diseases, such as MND, are known in the art (see, e.g., U.S. Application Publication No. 20120005765).

Methods for detecting, monitoring, quantifying or assessing motor deficits and histopathological deficiencies associated with MND are known in the art, including histopathological, biochemical, and electrophysiological studies and motor activity analysis (see, e.g., Rich et al. (2002) J. Neurophysiol. 88: 3293-3304; Appel et al., (1991) Proc. Natl. Acad. Sci. USA, 88: 647-51). Histopathologically, MNDs are characterized by death of motor neurons, progressive accumulation of detergent-resistant aggregates containing SOD1 and ubiquitin and aberrant neurofilament accumulations in degenerating motor neurons. In addition, reactive astroglia and microglia are often detected in diseased tissue. Patients with an MND show one or more motor deficits, including muscle weakness and wasting, uncontrollable twitching, spasticity, slow and effortful movements, and overactive tendon reflexes.

### Ophthalmic Diseases and Disorders

In certain embodiments, a senescence-associated disease or disorder is an ocular disease, disorder, or condition, for example, presbyopia, macular degeneration, or cataracts. In other certain embodiments, the senescence-associated disease or disorder is glaucoma. Macular degeneration is a neurodegenerative disease that causes the loss of photoreceptor cells in the central part of retina, called the macula. Macular degeneration generally is classified into two types: dry type and wet type. The dry form is more common than the wet, with about 90% of age-related macular degeneration (ARMD or AMD) patients diagnosed with the dry form. The wet form of the disease usually leads to more serious vision loss. While the exact causes of age-related macular degeneration are still unknown, the number of senescent retinal pigmented epithelial (RPE) cells increases with age. Age and certain genetic factors and environmental factors are risk factors for developing ARMD (see, e.g., Lyengar et al. (2004) Am. J. Hum. Genet. 74: 20-39); Kenealy et al. (2004) Mol. Vis. 10: 57-61; Gorin et al. (1999) Mol. Vis. 5: 29). Environment predisposing factors include omega-3 fatty acids intake (see, e.g., Christen et al. (2011) Arch Ophthalmol. 129: 921-929); estrogen exposure (see, e.g., Feshanich et al. (2008) Arch Ophthalmol. 126(4): 519-524); and increased serum levels of vitamin D (see, e.g., Millen, et al. (2011) Arch Ophthalmol. 129(4): 481-489). Genetic predisposing risk factors include reduced levels of Dicer1 (enzyme involved in maturation of micro RNA) in eyes of patients with dry AMD, and decreased micro RNAs contributes to a senescent cell profile; and DICER1 ablation induces premature senescence (see, e.g., Mudhasani et al. (2008) J. Cell Biol. 181(7): 1055-1063).

Dry ARMD is associated with atrophy of RPE layer, which causes loss of photoreceptor cells. The dry form of ARMD may result from aging and thinning of macular tissues and from deposition of pigment in the macula. Senescence appears to inhibit both replication and migration of RPE, resulting in permanent RPE depletion in the macula of dry AMD patients (see, e.g., Iriyama et al., J. Biol. Chem. 283:11947-953 (2008)). With wet ARMD, new blood vessels grow beneath the retina and leak blood and fluid. This abnormal leaky choroidal neovascularization causes the retinal cells to die, creating blind spots in central vision. Different forms of macular degeneration may also occur in younger patients. Non-age related etiology may be linked to heredity, diabetes, nutritional deficits, head injury, infection, or other factors.

Declining vision noticed by the patient or by an ophthalmologist during a routine eye exam may be the first indicator of macular degeneration. The formation of exudates, or “drusen,” underneath the Bruch's membrane of the macula is often the first physical sign that macular degeneration may develop. Symptoms include perceived distortion of straight lines and, in some cases, the center of vision appears more distorted than the rest of a scene; a dark, blurry area or “white-out” appears in the center of vision; and/or color perception changes or diminishes. Diagnosing and monitoring of a subject with macular degeneration may be accomplished by a person skilled in the ophthalmic art according to art-accepted periodic eye examination procedures and report of symptoms by the subject.

Presbyopia is an age-related condition where the eye exhibits a progressively diminished ability to focus on near objects as the speed and amplitude of accommodation of a normal eye decreases with advancing age. Loss of elasticity of the crystalline lens and loss of contractility of the ciliary muscles have been postulated as its cause (see, e.g., Heys et al., 2004, Mol. Vis. 10:956-63; Petrash, 2013, Invest. Ophthalmol. Vis. Sci. 54:ORSF54-ORSF59). Age-related changes in the mechanical properties of the anterior lens capsule and posterior lens capsule suggest that the mechanical strength of the posterior lens capsule decreases significantly with age (see, e.g., Krag et al., Invest. Ophthalmol. Vis. Sci. 44:691-96 (2003); Krag et al., Invest. Ophthalmol. Vis. Sci. 38:357-63 (1997)).

The laminated structure of the capsule also changes and may result, at least in part, from a change in the composition of the tissue (see, e.g., Krag et al., 1997, supra, and references cited therein). The major structural component of the lens capsule is basement membrane type IV collagen that is organized into a three-dimensional molecular network (see, e.g., Cummings et al., Connect. Tissue Res. 55:8-12 (2014); Veis et al., Coll. Relat. Res. 1981; 1:269-86). Type IV collagen is composed of six homologous a chains (α1-6) that associate into heterotrimeric collagen IV protomers with each comprising a specific chain combination of α112, a345, or a556 (see, e.g., Khoshnoodi et al., Microsc. Res. Tech. 2008; 71:357-70). Protomers share structural similarities of a triple-helical collagenous domain with the triplet peptide sequence of Gly-X-Y (Timpl et al., Eur. J. Biochem. 1979; 95:255-263), ending in a globular C-terminal region termed the non-collagenous 1 (NC1) domain. The N-termini are composed of a helical domain termed the 7S domain (see, e.g., Risteli et al., Eur. J. Biochem. 1980; 108:239-250), which is also involved in protomer-protomer interactions.

Research has suggested that collagen IV influences cellular function which is inferred from the positioning of basement membranes underneath epithelial layers, and data support the role of collagen IV in tissue stabilization (see, e.g., Cummings et al., supra). Posterior capsule opacification (PCO) develops as a complication in approximately 20-40% of patients in subsequent years after cataract surgery (see, e.g., Awasthi et al., Arch Ophthalmol. 2009; 127:555-62). PCO results from proliferation and activity of residual lens epithelial cells along the posterior capsule in a response akin to wound healing (see, e.g., Awasthi et al., Arch Ophthalmol. 2009; 127:555-62). Growth factors, such as fibroblast growth factor, transforming growth factor J, epidermal growth factor, hepatocyte growth factor, insulin-like growth factor, and interleukins IL-1 and IL-6 may also promote epithelial cell migration, (see, e.g., Awasthi et al., supra; Raj et al., supra). As discussed herein, production of these factors and cytokines by senescent cells contributes to the SASP. In contrast, in vitro studies show that collagen IV promotes adherence of lens epithelial cells (see, e.g., Olivero et al., Invest. Ophthalmol. Vis. Sci. 1993; 34:2825-34). Adhesion of the collagen IV, fibronectin, and laminin to the intraocular lens inhibits cell migration and may reduce the risk of PCO (see, e.g., Raj et al., Int. J. Biomed. Sci. 2007; 3:237-50).

Without wishing to be bound by any particular theory, selective killing of senescent cells by the senolytic agents described herein (e.g., CRYAB inhibitors) may slow or impede (delay, inhibit, retard) the disorganization of the type IV collagen network. Removal of senescent cells and thereby removal of the inflammatory effects of SASP may decrease or inhibit epithelial cell migration and may also delay (suppress) the onset of presbyopia or decrease or slow the progressive severity of the condition (such as slow the advancement from mild to moderate or moderate to severe). The senolytic agents described herein may also be useful for post-cataract surgery to reduce the likelihood of occurrence of PCO.

While no direct evidence for the involvement of cellular senescence with the development of cataracts has been obtained from human studies, BubR1 hypomorphic mice develop posterior subcapsular cataracts bilaterally early in life, suggesting that senescence may play a role (see, e.g., Baker et al., Nat. Cell Biol. 10:825-36 (2008)). Cataracts are a clouding of the lens of an eye, causing blurred vision, and if left untreated can result in blindness. Surgery is effective and routinely performed to remove cataracts. Administration of one or more of the senolytic agents described herein (e.g., CRYAB inhibitors) may result in decreasing the likelihood of occurrence of a cataract or may slow or inhibit progression of a cataract. The presence and severity of a cataract can be monitored by eye exams using methods routinely performed by a person skilled in the ophthalmology art.

In certain embodiments, at least one senolytic agent that selectively kills senescent cells (e.g. a CRYAB inhibitor) may be administered to a subject who is at risk of developing presbyopia, cataracts, or macular degeneration. Treatment with a senolytic agent may be initiated when a human subject is at least 40 years of age to delay or inhibit onset or development of cataracts, presbyopia, and macular degeneration. Because almost all humans develop presbyopia, in certain embodiments, the senolytic agent may be administered in a manner as described herein to a human subject after the subject reaches the age of 40 to delay or inhibit onset or development of presbyopia.

In certain embodiments, the senescence associated disease or disorder is glaucoma. Glaucoma is a broad term used to describe a group of diseases that causes visual field loss, often without any other prevailing symptoms. The lack of symptoms often leads to a delayed diagnosis of glaucoma until the terminal stages of the disease. Even if subjects afflicted with glaucoma do not become blind, their vision is often severely impaired. Normally, clear fluid flows into and out of the front part of the eye, known as the anterior chamber. In individuals who have open/wide-angle glaucoma, this fluid drains too slowly, leading to increased pressure within the eye. If left untreated, this high pressure subsequently damages the optic nerve and can lead to complete blindness. The loss of peripheral vision is caused by the death of ganglion cells in the retina. Ganglion cells are a specific type of projection neuron that connects the eye to the brain. When the cellular network required for the outflow of fluid was subjected to SA-β-Gal staining, a fourfold increase in senescence has been observed in glaucoma patients (see, e.g., Liton et al., Exp. Gerontol. 40:745-748 (2005)).

For monitoring the effect of a therapy on inhibiting progression of glaucoma, standard automated perimetry (visual field test) is the most widely used technique. In addition, several algorithms for progression detection have been developed (see, e.g., Wesselink et al., Arch Ophthalmol. 127(3):270-274 (2009), and references therein). Additional methods include gonioscopy (examines the trabecular meshwork and the angle where fluid drains out of the eye); imaging technology, for example scanning laser tomography (e.g., HRT3), laser polarimetry (e.g., GDX), and ocular coherence tomography); ophthalmoscopy; and pachymeter measurements that determine central corneal thickness.

### Metabolic Disease or Disorder.

Senescence-associated diseases or disorders treatable by administering a senolytic agent described herein (e.g., a CRYAB inhibitor) include metabolic diseases or disorders. Such senescent cell associated diseases and disorders include diabetes, metabolic syndrome, diabetic ulcers, and obesity.

Diabetes is characterized by high levels of blood glucose caused by defects in insulin production, insulin action, or both. The great majority (90 to 95%) of all diagnosed cases of diabetes in adults are type 2 diabetes, characterized by the gradual loss of insulin production by the pancreas. Diabetes is the leading cause of kidney failure, nontraumatic lower-limb amputations, and new cases of blindness among adults in the U.S. Diabetes is a major cause of heart disease and stroke and is the seventh leading cause of death in the U.S. (see, e.g., Centers for Disease Control and Prevention, National diabetes fact sheet: national estimates and general information on diabetes and pre-diabetes in the United States, 2011 (“Diabetes fact sheet”)). In certain embodiments, senolytic agents described herein (e.g., CRYAB inhibitors) may be used for treating type 2 diabetes, particularly age-, diet- and obesity-associated type 2 diabetes.

Involvement of senescent cells in metabolic disease, such as obesity and type 2 diabetes, has been suggested as a response to injury or metabolic dysfunction (see, e.g., Tchkonia et al., Aging Cell 9:667-684 (2010)). Fat tissue from obese mice showed induction of the senescence markers SA-β-Gal, p53, and p21 (see, e.g., Tchkonia et al., supra; Minamino et al., Nat. Med. 15:1082-1087 (2009)). A concomitant up-regulation of pro-inflammatory cytokines, such as tumor necrosis factor-α and Ccl2/MCP1, was observed in the same fat tissue (see, e.g., Minamino et al., supra). Induction of senescent cells in obesity potentially has clinical implications because pro-inflammatory SASP components are also suggested to contribute to type 2 diabetes (see, e.g., Tchkonia et al., supra). A similar pattern of up-regulation of senescence markers and SASP components are associated with diabetes, both in mice and in humans (see, e.g., Minamino et al., supra). Accordingly, the methods described herein that comprise administering a senolytic agent may be useful for treatment or prophylaxis of type 2 diabetes, as well as obesity and metabolic syndrome. Without wishing to be bound by theory, contact of senescent pre-adipocytes with a senolytic agent thereby killing the senescent pre-adipocytes may provide clinical and health benefit to a person who has any one of diabetes, obesity, or metabolic syndrome.

Subjects suffering from type 2 diabetes can be identified using standard diagnostic methods known in the art for type 2 diabetes. Generally, diagnosis of type 2 diabetes is based on symptoms (e.g., increased thirst and frequent urination, increased hunger, weight loss, fatigue, blurred vision, slow-healing sores or frequent infections, and/or areas of darkened skin), medical history, and/or physical examination of a patient. Subjects at risk of developing type 2 diabetes include those who have a family history of type 2 diabetes and those who have other risk factors such as excess weight, fat distribution, inactivity, race, age, prediabetes, and/or gestational diabetes.

The effectiveness of a senolytic agent can readily be determined by a person skilled in the medical and clinical arts. One or any combination of diagnostic methods, including physical examination, assessment and monitoring of clinical symptoms, and performance of analytical tests and methods, such as those described herein, may be used for monitoring the health status of the subject. A subject who is receiving one or more senolytic agents described herein for treatment or prophylaxis of diabetes can be monitored, for example, by assaying glucose and insulin tolerance, energy expenditure, body composition, fat tissue, skeletal muscle, and liver inflammation, and/or lipotoxicity (muscle and liver lipid by imaging in vivo and muscle, liver, bone marrow, and pancreatic β-cell lipid accumulation and inflammation by histology). Other characteristic features or phenotypes of type 2 diabetes are known and can be assayed as described herein and by using other methods and techniques known and routinely practiced in the art.

Obesity and obesity-related disorders are used to refer to conditions of subjects who have a body mass that is measurably greater than ideal for their height and frame. Body Mass Index (BMI) is a measurement tool used to determine excess body weight, and is calculated from the height and weight of a subject. A human is considered overweight when the person has a BMI of 25-29; a person is considered obese when the person has a BMI of 30-39, and a person is considered severely obese when the person has a BMI of ≥40. Accordingly, the terms obesity and obesity-related refer to human subjects with body mass index values of greater than 30, greater than 35, or greater than 40. A category of obesity not captured by BMI is called “abdominal obesity” in the art, which relates to the extra fat found around a subject's middle, which is an important factor in health, even independent of BMI. The simplest and most often used measure of abdominal obesity is waist size. Generally abdominal obesity in women is defined as a waist size 35 inches or higher, and in men as a waist size of 40 inches or higher. More complex methods for determining obesity require specialized equipment, such as magnetic resonance imaging or dual energy X-ray absorption metry machines.

A condition or disorder associated with diabetes and senescence is a diabetic ulcer (i.e., diabetic wound). An ulcer is a breakdown in the skin, which may extend to involve the subcutaneous tissue or even muscle or bone. These lesions occur, particularly, on the lower extremities. Patients with diabetic venous ulcer exhibit elevated presence of cellular senescence at sites of chronic wounds (see, e.g., Stanley et al. (2001) J. Vas. Surg. 33: 1206-1211). Chronic inflammation is also observed at sites of chronic wounds, such as diabetic ulcers (see, e.g., Goren et al. (2006) Am. J. Pathol. 7 168: 65-77; Seitz et al. (2010) Exp. Diabetes Res. 2010: 476969), suggesting that the proinflammatory cytokine phenotype of senescent cells has a role in the pathology.

Subjects who have type 2 diabetes or who are at risk of developing type 2 diabetes may have metabolic syndrome. Metabolic syndrome in humans is typically associated with obesity and characterized by one or more of cardiovascular disease, liver steatosis, hyperlipidemia, diabetes, and insulin resistance. A subject with metabolic syndrome may present with a cluster of metabolic disorders or abnormalities that may include, for example, one or more of hypertension, type-2 diabetes, hyperlipidemia, dyslipidemia (e.g., hypertriglyceridemia, hypercholesterolemia), insulin resistance, liver steatosis (steatohepatitis), hypertension, atherosclerosis, and other metabolic disorders.

### Renal Dysfunction

Nephrological pathologies, such as glomerular disease, arise in the elderly. Glomerulonephritis is characterized by inflammation of the kidney and by the expression of two proteins, IL1α and IL10 (see, e.g., Niemir et al. (1997) Kidney Int. 52:393-403). IL1α and IL10 are considered master regulators of SASP (see, e.g., Coppe et al. (2008) PLoS. Biol. 6: 2853-68). Glomerular disease is associated with elevated presence of senescent cells, especially in fibrotic kidneys (see, e.g., Sis et al. (2007) Kidney Int. 71:218-226).

### Dermatological Disease or Disorder.

Senescence-associated diseases or disorders treatable by administering a senolytic agent described herein (e.g., a CRYAB inhibitor) include dermatological diseases or disorders. Such senescent cell associated diseases and disorders include psoriasis and eczema, which are also inflammatory diseases and are discussed in greater detail above. Other dermatological diseases and disorders that are associated with senescence include rhytides (wrinkles due to aging); pruritis (linked to diabetes and aging); dysesthesia (chemotherapy side effect that is linked to diabetes and multiple sclerosis); psoriasis (as noted) and other papulosquamous disorders, for example, erythroderma, lichen planus, and lichenoid dermatosis; atopic dermatitis (a form of eczema and associated with inflammation); eczematous eruptions (often observed in aging patients and linked to side effects of certain drugs). Other dermatological diseases and disorders associated with senescence include eosinophilic dermatosis (linked to certain kinds of hemotologic cancers); reactive neutrophilic dermatosis (associated with underlying diseases such as inflammatory bowel syndrome); pemphigus (an autoimmune disease in which autoantibodies form against desmoglein); pemphigoid and other immunobullous dermatosis (autoimmune blistering of skin); fibrohistocytic proliferations of skin, which is linked to aging; and cutaneous lymphomas that are more common in older populations. Another dermatological disease that may be treatable according to the methods described herein includes cutaneous lupus, which is a symptom of lupus erythematosus. Late onset lupus may be linked to decreased (i.e., reduced) function of T-cell and B-cells and cytokines (immunosenescence) associated with aging.

### Metastasis.

In a particular embodiment, methods are provided for treating or preventing (i.e., reducing the likelihood of occurrence or development of) a senescence cell associated disease (or disorder or condition), which is metastasis. The senolytic agents described herein may also be used according to the methods described herein for treating or preventing (i.e., reducing the likelihood of occurrence of) metastasis (i.e., the spreading and dissemination of cancer or tumor cells) from one organ or tissue to another organ or tissue in the body.

A senescent cell-associated disease or disorder includes metastasis, and a subject who has a cancer may benefit from administration of a senolytic agent described herein (e.g., a CRYAB inhibitor) for inhibiting metastasis. Such a senolytic agent when administered to a subject who has a cancer according to the methods described herein may inhibit tumor proliferation. Metastasis of a cancer occurs when the cancer cells (i.e., tumor cells) spread beyond the anatomical site of origin and initial colonization to other areas throughout the body of the subject. Tumor proliferation may be determined by tumor size, which can be measured in various ways familiar to a person skilled in the art, such as by PET scanning, MRI, CAT scan, biopsy, for example. The effect of the therapeutic agent on tumor proliferation may also be evaluated by examining differentiation of the tumor cells.

As used herein and in the art, the terms cancer or tumor are clinically descriptive terms that encompass diseases typically characterized by cells exhibiting abnormal cellular proliferation. The term cancer is generally used to describe a malignant tumor or the disease state arising from the tumor. Alternatively, an abnormal growth may be referred to in the art as a neoplasm. The term tumor, such as in reference to a tissue, generally refers to any abnormal tissue growth that is characterized, at least in part, by excessive and abnormal cellular proliferation. A tumor may be metastatic and capable of spreading beyond its anatomical site of origin and initial colonization to other areas throughout the body of the subject. A cancer may comprise a solid tumor or may comprise a “liquid” tumor (e.g., leukemia and other blood cancers).

Cells are induced to senesce by cancer therapies, such as radiation and certain chemotherapy drugs. The presence of senescent cells increases secretion of inflammatory molecules (see description herein of senescent cells), promotes tumor progression, which may include promoting tumor growth and increasing tumor size, promoting metastasis, and altering differentiation. When senescent cells are destroyed, tumor progression is significantly inhibited, resulting in tumors of small size and with little or no observed metastatic growth (see, e.g., Int'l Appl. Publication No. WO 2013/090645).

In one embodiment, methods are provided for preventing (i.e., reducing the likelihood of occurrence of), inhibiting, or retarding metastasis in a subject who has a cancer by administering a senolytic agent as described herein. In a particular embodiment, a senolytic agent described herein (e.g., a CRYAB inhibitor) is administered on one or more days within a treatment window (i.e., treatment course) of no longer than 7 days or 14 days. In other embodiments, the treatment course is no longer than 2, 3, 4, 5, 6, 7, 8, 9, 10, 11, 12, 13, 14, 15, 16, 17, 18, 19, 20, or no longer than 21 days. In other embodiments, the treatment course is a single day. In certain embodiments, the senolytic agent is administered on two or more days within a treatment window of no longer than 7 days or 14 days, on 3 or more days within a treatment window of no longer than 7 days or 14 days; on 4 or more days within a treatment window of no longer than 7 days or 14 days; on 5 or more days within a treatment window of no longer than 7 days or 14 days; on 6, 7, 8, 9, 10, 11, 12, 13, or 14 days within a treatment window of no longer than 7 days or 14 days. In certain embodiments, when the at least one senolytic agent is administered to a subject for a treatment window of 3 days or more, the agent may be administered every 2^{nd} day (i.e., every other day). In other certain embodiments, when the at least one senolytic agent is administered to a subject for a treatment window of 4 days or more, the agent may be administered every 3^{rd} day (i.e., every other third day).

### Administration

In certain aspects, a prophylactically effective and/or a therapeutically effective amount of one or more CRYAB inhibitors (e.g., as described herein) may be administered to a subject. Administration is performed using standard effective techniques, including peripherally (i.e., not by administration into the central nervous system) or locally to the central nervous system. Peripheral administration includes but is not limited to oral, inhalation, intravenous, intraperitoneal, intra-articular, subcutaneous, pulmonary, transdermal, intramuscular, intranasal, buccal, sublingual, or suppository administration. Local administration, including directly into the central nervous system (CNS) includes but is not limited to via a lumbar, intraventricular or intraparenchymal catheter or using a surgically implanted controlled release formulation. The route of administration may be dictated by the disease or condition to be treated. For example, if the disease or condition is COPD or IPF, the composition may be administered via inhalation. Alternatively, if the disease or condition is osteoarthritis, the composition may be administered via intra-articular administration. It is within the skill of one in the art, to determine the route of administration based on the disease or condition to be treated. In a specific embodiment, a composition of the invention is administered orally.

Pharmaceutical compositions comprising one or more CRYAB inhibitors for effective administration are deliberately designed to be appropriate for the selected mode of administration, and pharmaceutically acceptable carriers such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners.

For therapeutic applications, a therapeutically effective amount of one or more CRYAB inhibitor(s) or a composition comprising one or more CRYAB inhibitor(s) is administered to a subject. A “therapeutically effective amount” is an amount of the therapeutic composition sufficient to produce a measurable response (e.g., cell death of senescent cells, an anti-aging response, a delay in the onset of or progression of or improvement in symptoms associated with a degenerative disease, a delay in the onset of or progression of or an improvement in symptoms associated with a function-decreasing disorder, or a delay in the onset of or progression of or improvement in symptoms associated with a DNA damaging therapy). Actual dosage levels of active ingredients in a therapeutic composition of the invention can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, age, the age-related disease or condition, the degenerative disease, the function-decreasing disorder, the symptoms, and the physical condition and prior medical history of the subject being treated. In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

In various embodiments, the frequency of dosing may be daily or once, twice, three times or more per week or per month, as needed for prophylactic effect or as to effectively treat the symptoms. The timing of administration of the treatment relative to the disease itself and duration of treatment will be determined by the circumstances surrounding the case. Treatment could begin immediately, such as at the site of the injury as administered by emergency medical personnel. Treatment could begin in a hospital or clinic itself, or at a later time after discharge from the hospital or after being seen in an outpatient clinic. Duration of treatment could range from a single dose administered on a one-time basis to a life-long course of therapeutic treatments.

Dosages of one or more CRYAB inhibitor(s) can vary between wide limits, depending upon the disease or disorder to be treated, the age and condition of the subject to be treated. In an embodiment where a composition comprising a CRYAB inhibitor is contacted with a sample, the concentration of the CRYAB inhibitor may be from about 1 μM to about 1000 μM. In certain embodiments, the concentration of the CRYAB inhibitor may be from about 5 μM to about 25 μM. For example, the concentration of the CRYAB inhibitor may be about 1, about 2.5 about 5, about 6, about 7, about 8, about 9, about 10, about 11, about 12, about 13, about 14, about 15, about 16, about 17, about 18, about 19, about 20, about 21, about 22, about 23, about 24, about 25, about 30, about 35, or about 40 μM. Additionally, the concentration of the CRYAB inhibitor may be greater than 40 μM. For example, the concentration of CRYAB inhibitor may be about 40, about 45, about 50, about 55, about 60, about 65, about 70, about 75, about 80, about 85, about 90, about 95 or about 100 μM.

In certain embodiments, the composition comprising a CRYAB inhibitor may be from about 0.1 mg/kg to about 500 mg/kg. For example, the dose of a CRYAB inhibitor may be about 0.1 mg/kg, about 0.5 mg/kg, about 1 mg/kg, about 5 mg/kg, about 10 mg/kg, about 15 mg/kg, about 20 mg/kg, or about 25 mg/kg. Alternatively, the dose of the CRYAB inhibitor may be about 25 mg/kg, about 50 mg/kg, about 75 mg/kg, about 100 mg/kg, about 125 mg/kg, about 150 mg/kg, about 175 mg/kg, about 200 mg/kg, about 225 mg/kg, or about 250 mg/kg. Additionally, the dose of the CRYAB inhibitor may be about 300 mg/kg, about 325 mg/kg, about 350 mg/kg, about 375 mg/kg, about 400 mg/kg, about 425 mg/kg, about 450 mg/kg, about 475 mg/kg or about 500 mg/kg.

Typical dosage levels can be determined and optimized using standard clinical techniques and will be dependent on the mode of administration.

### Subject

A subject may be a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

The human subject may be of any age. However, since senescent cells are normally associated with aging, a human subject may be an older human subject. In some embodiments, the human subject may be about 30, 35, 40, 45, 50, 55, 60, 65, 70, 75, 80, 85, 90, 95 years of age or older. In some preferred embodiments, the human subject is 30 years of age or older. In other preferred embodiments, the human subject is 40 years of age or older. In other preferred embodiments, the human subject is 45 years of age or older. In yet other preferred embodiments, the human subject is 50 years of age or older. In still other preferred embodiments, the human subject is 55 years of age or older. In other preferred embodiments, the human subject is 60 years of age or older. In yet other preferred embodiments, the human subject is 65 years of age or older. In still other preferred embodiments, the human subject is 70 years of age or older. In other preferred embodiments, the human subject is 75 years of age or older. In still other preferred embodiments, the human subject is 80 years of age or older. In yet other preferred embodiments, the human subject is 85 years of age or older. In still other preferred embodiments, the human subject is 90 years of age or older.

Additionally, a subject in need thereof may be a subject suffering from an age-related disease or condition as described above.

### Pharmaceutical Formulations.

In various embodiments, pharmaceutical formulations comprising one or more agents that inhibit expression and/or aggregation of CRYAB are provided. In certain embodiments, the pharmaceutical formulation comprises agents that inhibit expression and/or aggregation of CRYAB and at least one pharmaceutically acceptable carrier. “pharmaceutically acceptable carrier”.

In various embodiments illustrative, but non-limiting embodiments, the pharmaceutically acceptable carrier may comprise a diluent, a binder, a filler, a buffering agent, a pH modifying agent, a disintegrant, a dispersant, a preservative, a lubricant, taste-masking agent, a flavoring agent, and/or a coloring agent. The amount and types of carriers utilized to form pharmaceutical compositions may be selected according to known principles of pharmaceutical science.

In one illustrative, but non-limiting embodiment, the carrier may comprise a diluent. In various embodiments, the diluent may be compressible (i.e., plastically deformable) or abrasively brittle. Non-limiting examples of suitable compressible diluents include microcrystalline cellulose (MCC), cellulose derivatives, cellulose powder, cellulose esters (e.g., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In certain embodiments, the carrier may comprise a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

In certain embodiments, the carrier may comprise a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In certain embodiments, the carrier may comprise a buffering agent. Representative examples of suitable buffering agents include, but are not limited to, phosphates, carbonates, citrates, tris buffers, and buffered saline salts (e.g., Tris buffered saline or phosphate buffered saline, and the like).

In various embodiments, the carrier may comprise a pH modifier. By way of non-limiting example, in certain embodiments, the pH modifying agent may comprise sodium carbonate, sodium bicarbonate, sodium citrate, citric acid, or phosphoric acid.

In certain embodiments, the carrier may comprise a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In certain embodiments, the carrier may comprise a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In certain embodiments, the carrier may comprise a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; antimicrobials, such as parabens, chlorobutanol, or phenol; and the like.

In certain embodiments, the carrier may comprise be a lubricant. Non-limiting examples of suitable lubricants include minerals such as talc or silica and/or fats such as vegetable stearin, magnesium stearate or stearic acid.

In certain embodiments, the carrier may comprise a taste-masking agent. Taste-masking materials include cellulose ethers, polyethylene glycols, polyvinyl alcohol, polyvinyl alcohol and polyethylene glycol copolymers, monoglycerides or triglycerides, acrylic polymers, mixtures of acrylic polymers with cellulose ethers, cellulose acetate phthalate, and combinations thereof.

In certain embodiments, the carrier may comprise a flavoring agent. In certain embodiments, flavoring agents may be chosen from synthetic flavor oils and flavoring aromatics and/or natural oils, extracts from plants, leaves, flowers, fruits, and combinations thereof.

In certain embodiments, the carrier may comprise a coloring agent. Suitable color additives include, but are not limited to, food, drug and cosmetic colors (FD&C), drug and cosmetic colors (D&C), or external drug and cosmetic colors (Ext. D&C).

In certain embodiments, the weight fraction of the carrier or combination of carriers in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

In certain embodiments, composition can be formulated into various dosage forms and administered by a number of different means that will deliver a therapeutically effective or prophylactically effective amount of the active ingredient(s) (CRYAB inhibitor(s)). Such compositions can be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18′ ed, 1995), and Liberman, H. A. and Lachman, L., Eds., Pharmaceutical Dosage Forms, Marcel Dekker Inc., New York, N.Y. (1980). In one embodiment, the composition may comprise a food supplement or the composition may comprise a cosmetic.

Solid dosage forms for oral administration include capsules, tablets, caplets, pills, powders, pellets, and granules. In such solid dosage forms, the active ingredient is ordinarily combined with one or more pharmaceutically acceptable carriers, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use.

In certain embodiments, extemporaneous injection solutions and suspensions may be prepared from sterile powders, granules and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising a CRYAB inhibitor, e.g., as described herein, is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering one or more CRYAB inhibitors described herein. Non-limiting examples of suitable structured fluid delivery systems may include nanoparticles, liposomes, microemulsions, micelles, dendrimers and other phospholipid-containing systems. Methods of incorporating compositions into delivery vehicles are known in the art.

In one illustrative embodiment, a liposome delivery vehicle may be utilized. Liposomes, depending upon the embodiment, are suitable for delivery of the CRYAB inhibitor(s) described herein in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the CRYAB inhibitor(s) may be selectively delivered to a cell by encapsulation in a liposome that fuses with the targeted cell's membrane.

Liposomes may be comprised of a variety of different types of phospholipids having varying hydrocarbon chain lengths. Phospholipids generally comprise two fatty acids linked through glycerol phosphate to one of a variety of polar groups. Suitable phospholipids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9,12,15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

In certain embodiments, the phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

In certain embodiments, the liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). Illustrative PEGs may range in size from about 500 to about 10,000 daltons.

In certain embodiments, liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying one or more CRYAB inhibitors may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, one or more CRYAB inhibitor(s) may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. One or more CRYAB inhibitor(s) may be encapsulated in a microemulsion by any method generally known in the art.

In certain embodiments, the one or more CRYAB inhibitor(s) described herein may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

The foregoing methods, compositions, and formulations are illustrative and not limiting. Using the teaching provided herein numerous other methods, compositions, and formulations will be available to one of skill in the art.

Some animal models which are related to senescence-associated diseases or disorders are already well known in the art. For example, a mouse model related to Osteoarthritis which is one of the senescence-associated diseases is disclosed (Jeon O H et al. (2017) Nat Med 23(6): 775-781). Other mouse models related to senescence-associated diseases or disorders are also well known in the art, for example, progeroid model mice (Baker et al. (2011) Nature, 479(7372): 232-236; Zhang et al. (2017) Redox Biol. 11: 30-37), aged model mice (Baker et al. (2016) Nature, 530(7589): 184-189; Chang et al. (2016) Nat. Med. 22(1): 78-83; Xu et al. (2018) Nat. Med. 24(8): 1246-1256), irradiation-induced side-effect model mice (Chang et al. (2016) Nat. Med. 22(1): 78-83), chemotherapy-induced side effect model mice (Demaria et al. (2017) Cancer Discov, 7(2): 165-176; Baar et al. (2017) Cell, 169(1): 132-147.e16), idiopathic pulmonary fibrosis model mice (Schafer et al. (2017) Nat. Commun. 8: 14532; Wiley et al. (2019) JCI Insight, 4(24): 130056), experimental ocular hypertension model mice (Rocha et al. (2019) Aging Cell, 19(2): e13089), Parkinson's disease model mice (Chinta et al. (2018) Cell Rep. 22(4): 930-940), Alzheimer's disease model mice (Wei et al. (2016) Chin. Med. J. 129(15): 1835-44; Zhang et al. (2019) Nat. Neurosci. 22(5): 719-728), tau-dependent neurodegenerative model mice (Bussian et al. (2018) Nature, 562(7728): 578-582), diabetes and hepatic steatosis model mice (Ogrodnik et al. (2017) Nat. Commun. 8: 15691; Aguayo-Mazzucato et al. (2019) Cell Metab. 30(1): 129-142.e4) and atherosclerosis model mice (Childs et al. (2016) Science, 354(6311):472-477). These animal models can be used to investigate the effectiveness of a senolytic agent (e.g., a CRYAB inhibitor).

### EXAMPLES

The following examples are offered to illustrate, but not to limit the claimed invention.

### Example 1

### The Crystallin Alpha B (CRYAB) Gene and Gene Product as Targets for Senolytic

### Agents

To identify genes uniquely associated with senescence in skeletal muscle, specific cell populations purified from skeletal muscle, satellite cells, and fibro adipogenic progenitors (FAPs) were focused on, and analyzed for genes uniquely associated with either senescence, or the SASP in small populations of cells. Single cell profiling of these cells was used to identify genes associated with senescence. A number of novel potential senolytic target genes were identified using this approach.

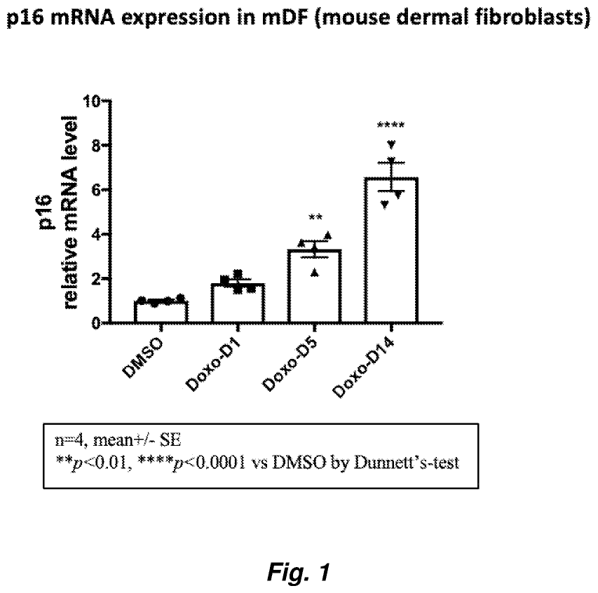

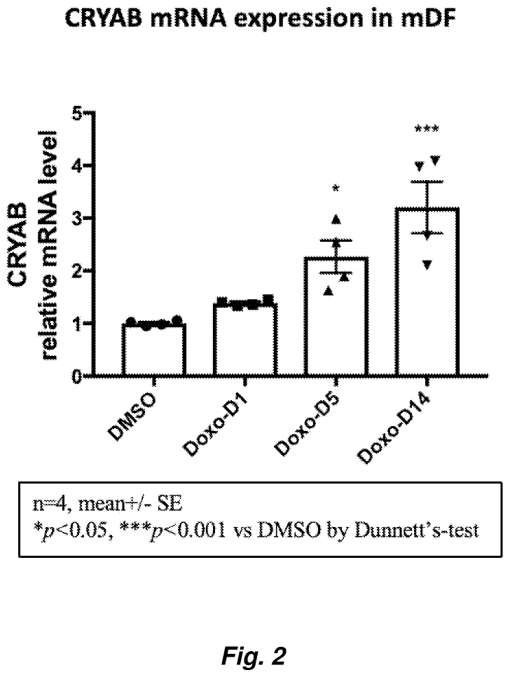

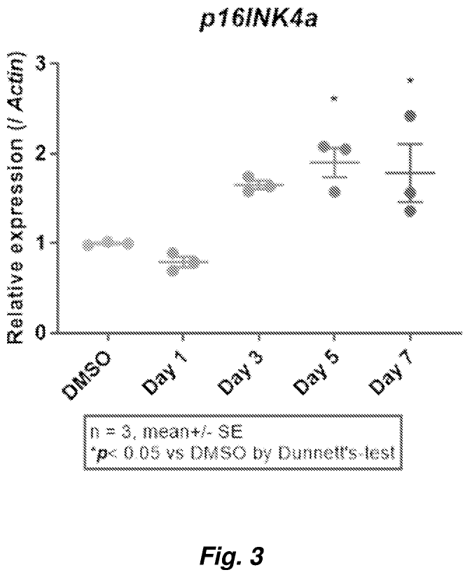

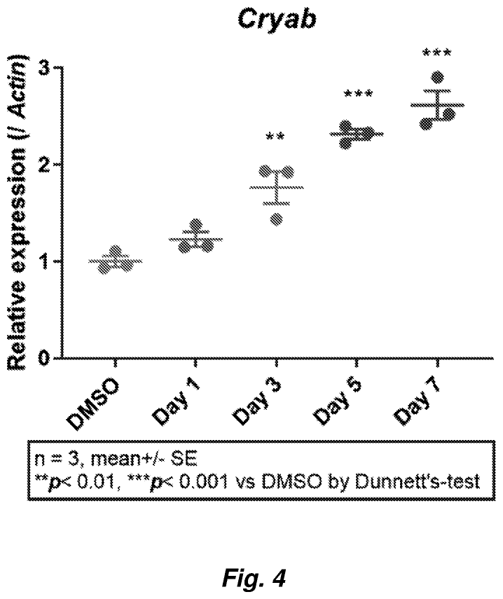

Target gene validation was then performed in cell culture. Senescence induction using doxorubicin (Doxo) in cell cultures of mouse dermal fibroblasts (mDFs) and fibro adipogenic progenitors (FAPs) (see, e.g., FIGS. 1 and 3), FAPs and Satellite cells was used to validate gene modulation of selected potential senolytic target genes in senescent cells (SEN) compared to non-senescent (NS) cells. Using this approach, CRYAB (crystalliln alpha B) was identified as a potential target gene and it was observed that senescent mDFs and FAPs upregulated CRYAB (see, e.g., FIGS. 2 and 4).

Using shRNA approach, the potential senolytic target genes were knocked down in cell culture to test if the knockdown of the selected target would kill the senescent cells. Small molecule inhibitors for the target genes, were validated as senolytic-targets using shRNA approach. The senolytic capability of the selected small molecule inhibitors was then tested.

As described below, the crystallin alpha B (CRYAB) gene and gene product were identified as a novel target gene for senolytics.

In particular, cells were treated with doxorubicin (Doxo) to induce senescence and/or with ABT263 (Navitoclax, 4-(4-{[2-(4-Chlorophenyl)-5,5-dimethyl-1-cyclohexen-1-yl]methyl}-1-piperazinyl)-N-[(4-{[(2R)-4-(4-morpholinyl)-1-(phenylsulfanyl)-2-butanyl]amino}-3-[(trifluoromethyl)sulfonyl]phenyl)sulfonyl]benzamide) to eliminate senescent cells (see, e.g., FIG. 1). Genetic techniques were used to inactivate candidate genes associated with senescence and cell survival in response to Doxo, and to look for specific senescent cell killing (“senolysis”) via down regulating the target genes using conventional molecular techniques. One such gene identified using this approach was the crystallin alpha B (CRYAB) gene, which appears to be specifically induced by senescence. Genetic knockdown of this gene kills senescent cells.

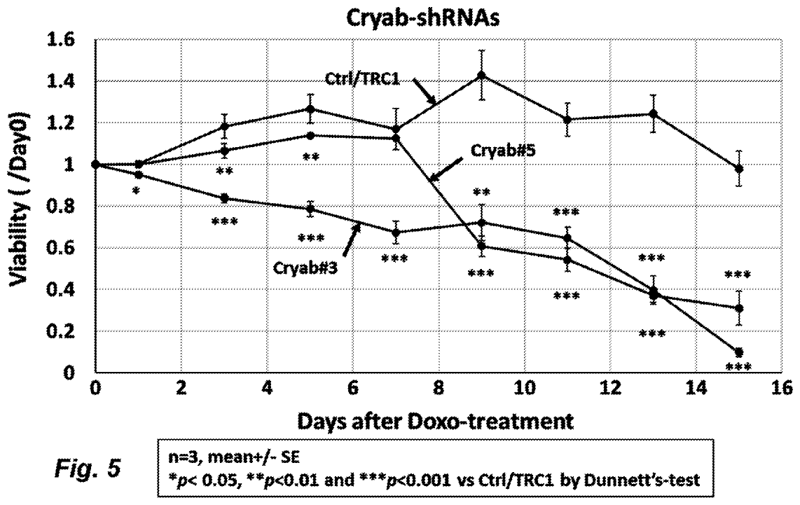

FIG. 5 shows that the suppressed expression of CRYAB significantly decreased the cell viability of senescent FAPs.

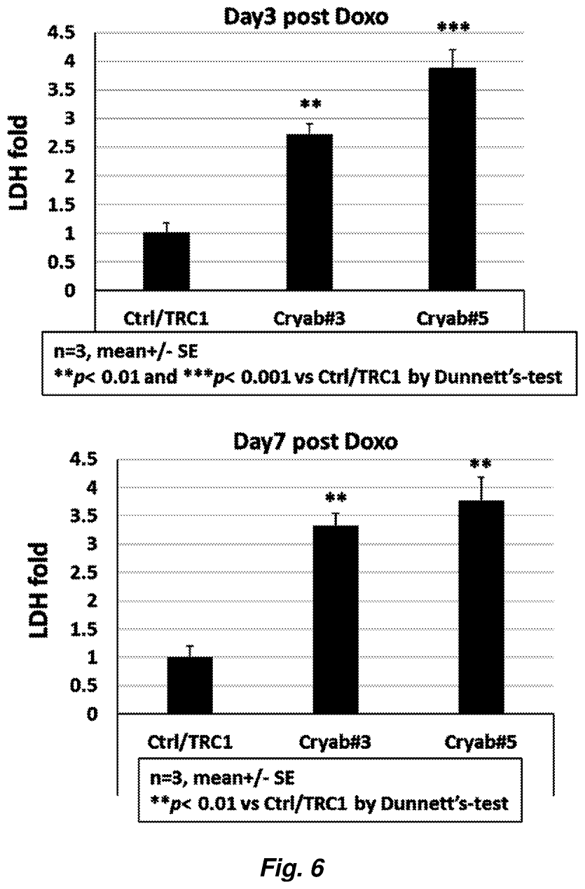

FIG. 6 shows that the suppressed expression of CRYAB significantly increased cytotoxity of senescent FAPs.

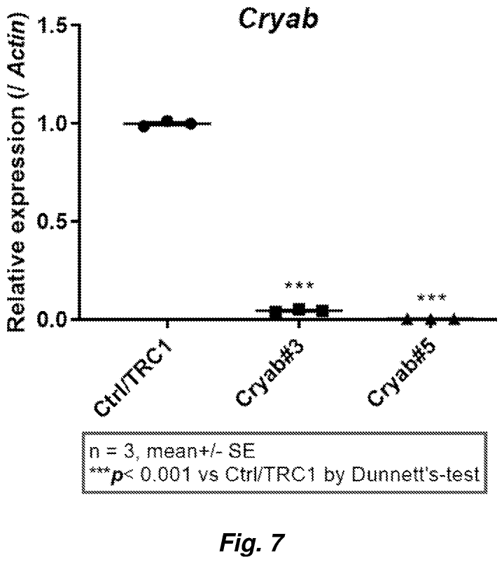

FIG. 7 shows that the shRNAs for CRYAB drastically suppressed CRYAB expression in FAPs.

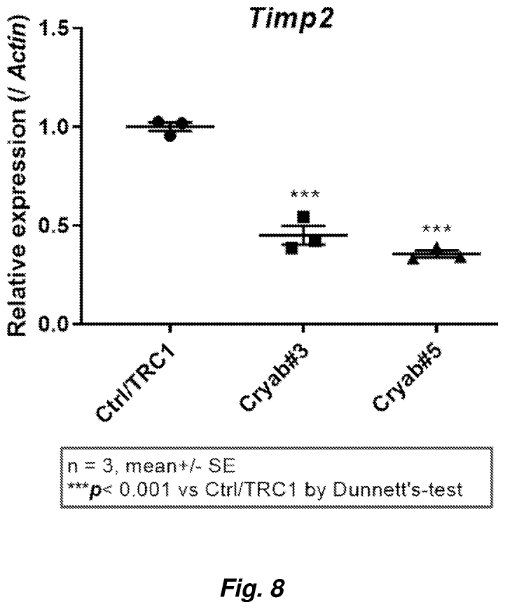

FIG. 8 shows that the suppressed expression of CRYAB significantly decreased Timp2 expression in senescent FAPs.

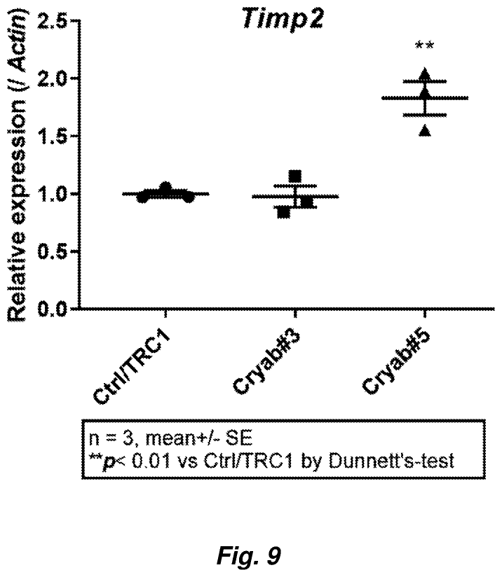

FIG. 9 shows that the suppressed expression of CRYAB didn't decrease Timp2 expression in non-senescent FAPs.

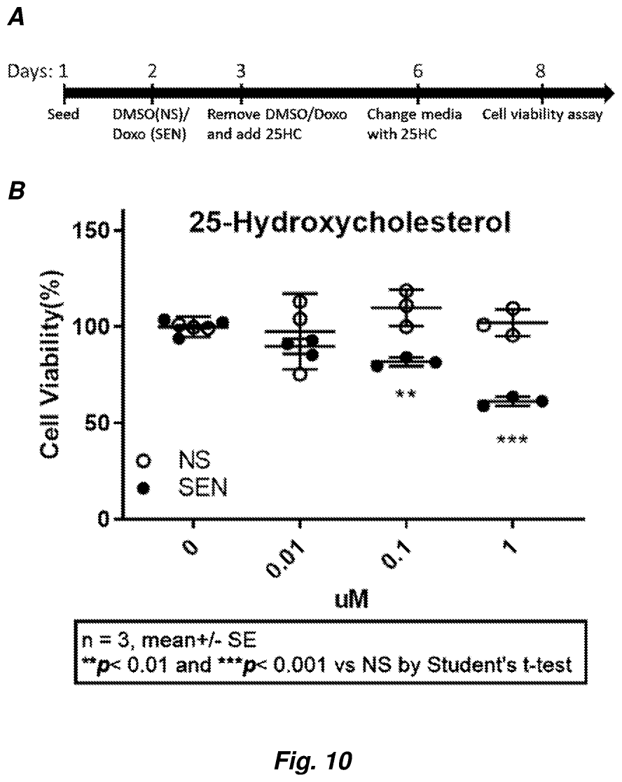

FIG. 10, panel A-B, shows that 25HC treatment specifically kills senescent FAPs at early-timepoint after senescence induction.

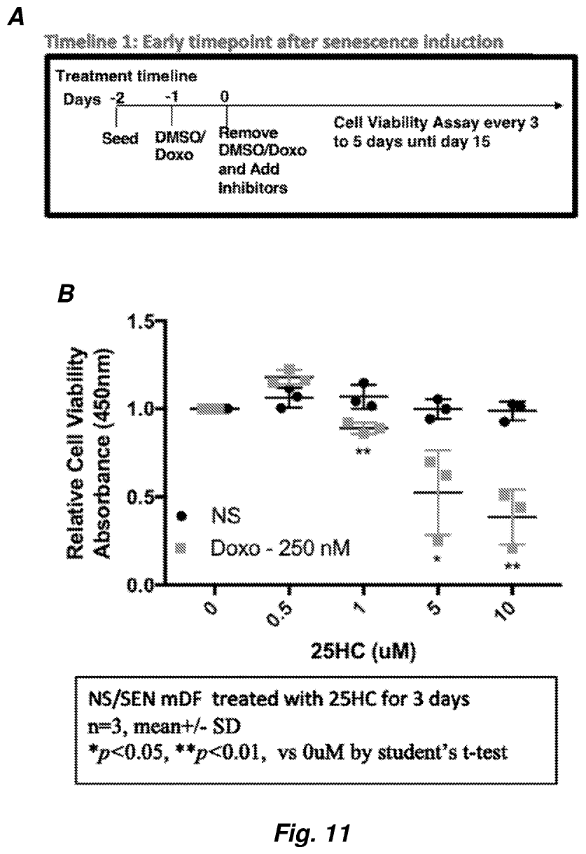

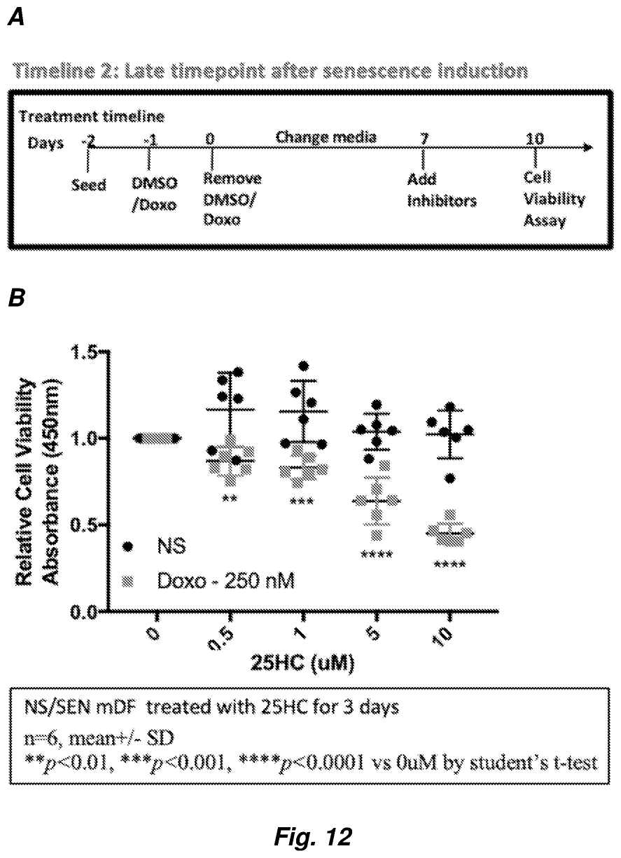

FIG. 11, panels A-B, shows that 25HC treatment specifically kills senescent mDFs at early-timepoint after senescence induction, while FIG. 12, panels A-B, shows that 25HC treatment specifically kills senescent mDFs at late-timepoint after senescence induction.

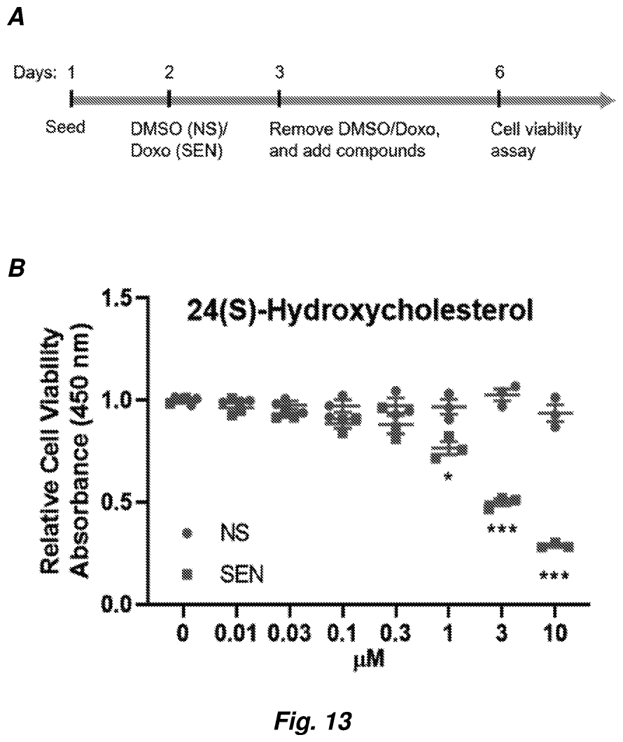

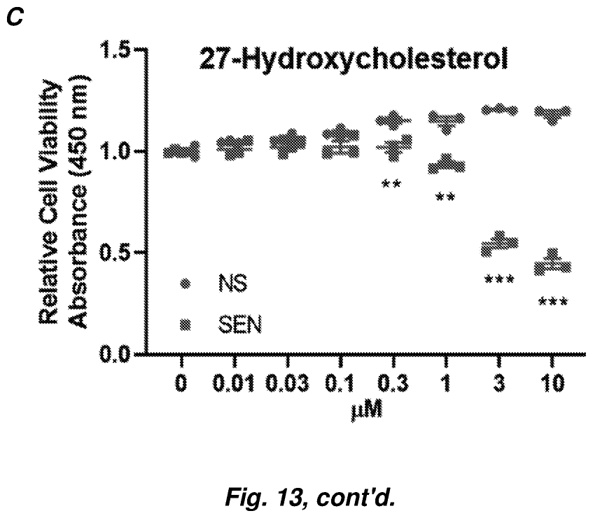

FIG. 13, panels A-C, shows that treatments of 24(S)HC and 27HC specifically kill senescent IMR-90 cells at early-timepoint after senescence induction.

These data thus show that inhibition of CRYAB protein aggregation and by implication, inhibition of CRYAB expression is an effective mechanism to selectively induce killing of senescent cells.

### Example 2

### Reduction or Elimination of Senescent Cells Improves (Decreases) Vascular Stiffness.

To evaluate whether age-related vascular stiffness could be reversed by pharmacologically eliminating senescent cells, we compared pulsed wave velocity (PWV) of the aorta in two paradigms: 1) Genetic elimination of senescent cells using the p16-3MR mouse; and 2) treatment with 25-hydroxycholesterol (25HC) at 50 mpk (IP).

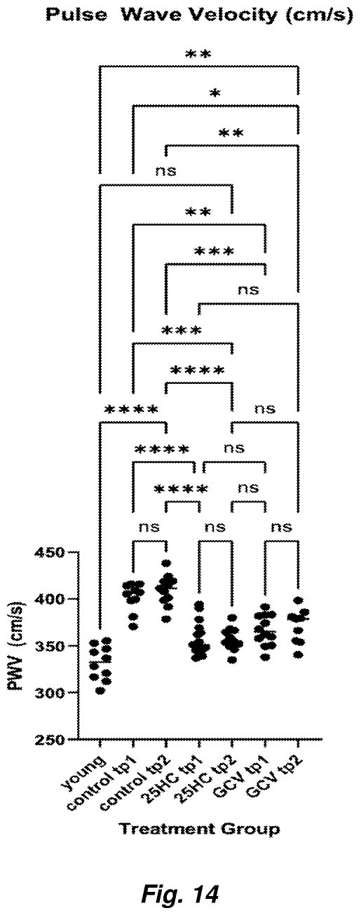

At baseline, animals were evaluated for blood flow in the aorta using doppler imaging. This determines a measure of vascular stiffness with vessels being more stiff having faster flow. Robust differences at baseline were found between young versus old mice (female, C57BL/6J, 3MR mice at 6 months of age versus 27 months of age (see, e.g., FIG. 14) consistent with prior studies. We then treated groups of mice with 25HC, or ganciclovir (GCV) (to induce Senolysis in the aged mice).

We evaluated PWV at 28 months of age (1 month post treatment (TP1)), and 29 months of age (2 months post treatment—TP2). Highly statistically significant reductions in PWV were seen in both groups compared to untreated controls (see, e.g., FIG. 14). This represents strong evidence for vascular stiffness being in part derived from cellular senescence, and eliminating senescent cells improving vascular function.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of preventing or slowing an increase in vascular stiffness or of decreasing vascular stiffness in a subject, said method comprising:
administering to said subject an effective amount of a senolytic agent.

2. (canceled)

3. The method of claim 1, wherein said method slows or prevents an increase in arterial stiffness or aortic stiffness in a subject.

4.-6. (canceled)

7. The method of claim 1, wherein said method decreases arterial stiffness or aortic stiffness in a subject.

8.-11. (canceled)

12. The method of claim 1, wherein said method improves Windkessel effect, and/or reduces load on heart, and/or lowers resting heart rate, and/or lowers duration of systole with an accompanying increase in duration of diastole in said subject.

13. (canceled)

14. The method of claim 1, wherein said senolytic agent is a senolytic agent that inhibits expression and/or aggregation of a CRYAB protein, wherein said senolytic agent is selected from the group consisting of a small organic molecule, an inhibitory nucleic acid, an antibody, a CRISPR/Cas system, a zinc finger nuclease (ZFN), and a transcription activator-like effector nuclease (TALEN).

15. (canceled)

16. (canceled)

17. The method of claim 1, wherein said senolytic agent is selected from the group consisting of 25-hydroxycholesterol (25HC), 24(S)-Hydroxycholesterol (24(S)HC), 27-Hydroxycholesterol (27HC), 22(R)-Hydroxycholesterol (22(R)HC), 7α-Hydroxycholesterol (7αHC), 7β-Hydroxycholesterol (7βHC), Calcifediol 25-Hydroxyvitamin D3, 7α,25-Dihydroxycholesterol, (3S,10R,13R)-17-(5-(dimethylamino)pentan-2-yl)-10,13-dimethyl-2,3,4,7,8,9,10,11,12,13,14,15,16,17-tetradecahydro-1H-cyclopenta[a]phenanthren-3-ol, rel-(3R,10S,13S)-17-[(2S)-6-hydroxy-6-phenylheptan-2-yl]-10,13-dimethyl-2,3,4,7,8,9,10,11,12,13,14,15,16,17-tetradecahydro-1H-cyclopenta[a]phenanthren-3-ol, 27-Nor-25-ketocholesterol and (3alpha,9xi,14xi)-3-hydroxychol-5-en-24-oic acid, or a salt thereof.

18. (canceled)

19. (canceled)

20. The method of claim 1, wherein said senolytic agent comprises a molecular tweezers that inhibits aggregation of a CRYAB protein.

21. (canceled)

22. (canceled)

23. The method of claim 1, wherein said senolytic agent comprises an inhibitory nucleic acid selected from the group consisting of an interfering RNA molecule (e.g., shRNA or siRNA), dsRNA, RNA polymerase III transcribed DNA, antisense nucleic acids, and a ribozyme.

24. The method of claim 1, wherein said senolytic agent comprises an shRNA or an siRNA that inhibits CRYAB expression.

25. The method of claim 1, wherein said senolytic agent comprises a CRISPR/Cas system that targets CRYAB, a zinc finger nuclease (ZFN) that targets CRYAB, or a transcription activator-like effector nuclease (TALEN) that targets CRYAB.

26. The method of claim 1, wherein said senolytic agent comprises an antibody that binds to a CRYAB protein.

27. The method of claim 1, wherein said senolytic agent is a small molecule senolytic agent that selectively kills senescent cells in comparison with non-senescent cells.

28. The method of claim 27, wherein the senolytic agent is a specific inhibitor of MDM2, Bcl-xL or Akt.

29.-31. (canceled)

32. The method of claim 28, wherein the MDM2 inhibitor is selected from the group consisting of Nutlin-1, Nutlin-2, Nutlin-3, RG-7112, RG7388, RO5503781, DS-3032b, MI-63, MI-126, MI-122, MI-142, MI-147, MI-18, MI-219, MI-220, MI-221, MI-773, 3-(4-chlorophenyl)-34(1-(hydroxymethyl)cyclopropyl)methoxy)-2-(4-nitrobenzyl)isoindolin-1-one, Serdemetan, AM-8553, CGM097, RO-2443, and RO-5963.

33. (canceled)

34. The method of claim 1, wherein the senolytic agent comprises an imidazoline compound, wherein the imidazoline compound comprises a compound having the structure: 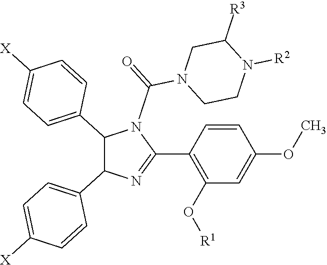
or a pharmaceutically acceptable salt thereof; wherein:
X is halide;
R¹ is alkyl, R² is —H or heteroalkyl, and
R³ is —H or ═O.

35. (canceled)

36. (canceled)

37. The method of claim 34, wherein the imidazoline compound comprises a compound having the structure: 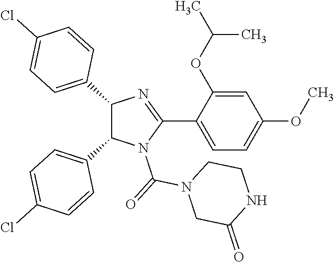
or a pharmaceutically acceptable salt thereof.

38. The method of claim 1, wherein said agent is administered systemically, topically, transdermally, intradermally, intranasally, by inhalation, intratracheally, or by intubation.

39.-42. (canceled)

43. The method of claim 1, wherein said subject, when administered said senolytic agent is not diagnosed with and/or under treatment for a pathology associated with aggregation proteins other than CRYAB.

44.-48. (canceled)

49. The method of claim 1, wherein said subject is not under treatment for a neurological pathology or an ophthalmic disorder.

50.-52. (canceled)

53. The method of claim 1, wherein said subject is not under treatment for a condition selected from the group consisting of Alzheimer's disease and related dementias, amyloid or other cause-mediated mild cognitive impairment (MCI), brain or spinal cord injury (including, but not limited to stroke), Huntingtin's disease, and Parkinson's disease.

54.-60. (canceled)


SEQUENCE LISTING (23 sequences)

SEQ ID NO: 1-6 - skipped (6 entries)

SEQ ID NO: 7 - RNA, 19 nt, synthetic construct
  misc_feature 1..19: note = Cpf1 guide RNA
  source 1..19: mol_type = other RNA
aatttctactgttgtagat

SEQ ID NO: 8 - RNA, 19 nt, synthetic construct
  misc_feature 1..19: note = Cpf1 guide RNA
  source 1..19: mol_type = other RNA
aatttctgctgttgcagat

SEQ ID NO: 9 - RNA, 19 nt, synthetic construct
  misc_feature 1..19: note = Cpf1 guide RNA
  source 1..19: mol_type = other RNA
aatttccactgttgtggat

SEQ ID NO: 10 - RNA, 19 nt, synthetic construct
  misc_feature 1..19: note = Cpf1 guide RNA
  source 1..19: mol_type = other RNA
aattcctactgttgtaggt

SEQ ID NO: 11 - RNA, 19 nt, synthetic construct
  misc_feature 1..19: note = Cpf1 guide RNA
  source 1..19: mol_type = other RNA
aatttctactattgtagat

SEQ ID NO: 12 - RNA, 19 nt, synthetic construct
  misc_feature 1..19: note = Cpf1 guide RNA
  source 1..19: mol_type = other RNA
aatttctactgctgtagat

SEQ ID NO: 13 - RNA, 18 nt, synthetic construct
  misc_feature 1..18: note = Cpf1 guide RNA
  source 1..18: mol_type = other RNA
aatttctactttgtagat

SEQ ID NO: 14 - RNA, 17 nt, synthetic construct
  misc_feature 1..17: note = Cpf1 guide RNA
  source 1..17: mol_type = other RNA
aatttctacttgtagat

SEQ ID NO: 15 - protein, 7 aa, synthetic construct
  REGION 1..7: note = Peptide comprising nuclear localization signal (NLS)
PKKKRKV

SEQ ID NO: 16 - protein, 7 aa, synthetic construct
  REGION 1..7: note = Peptide comprising nuclear localization signal (NLS)
PKKKRRV

SEQ ID NO: 17 - protein, 16 aa, synthetic construct
  REGION 1..16: note = Peptide comprising nuclear localization signal (NLS)
KRPAATKKAGQAKKKK

SEQ ID NO: 18 - protein, 20 aa, synthetic construct
  REGION 1..20: note = Cell penetrating peptide (CPP)
GRKKRRQRRRPPQPKKKRKV

SEQ ID NO: 19 - protein, 19 aa, synthetic construct
  REGION 1..19: note = Cell penetrating peptide (CPP)
PLSSIFSRIGDPPKKKRKV

SEQ ID NO: 20 - protein, 24 aa, synthetic construct
  REGION 1..24: note = Cell penetrating peptide (CPP)
GALFLGWLGAAGSTMGAPKKKRKV

SEQ ID NO: 21 - protein, 27 aa, synthetic construct
  REGION 1..27: note = Cell penetrating peptide (CPP)
GALFLGFLGAAGSTMGAWSQPKKKRKV

SEQ ID NO: 22 - protein, 21 aa, synthetic construct
  REGION 1..21: note = Cell penetrating peptide (CPP)
KETWWETWWTEWSQPKKKRKV

SEQ ID NO: 23 - protein, 175 aa, Homo sapiens
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKK